(19) United States
(12) Patent Application Publication
(10) Pub. No.: US 2026/0295093 A1
(43) Pub. Date: Oct. 1, 2026

(54) GENE THERAPY FOR TREATMENT OF PROTEIN MISFOLDING DISEASES

(71) Applicant: RESEARCH INSTITUTE AT NATIONWIDE CHILDREN'S HOSPITAL, Columbus, OH (US)

(72) Inventor: Zarife Sahenk, Columbus, OH (US)

(21) Appl. No.: 19/480,491

(22) PCT Filed: May 2, 2024
(86) PCT No.: PCT/US2024/027477

§ 371 Date: Oct. 31, 2025

Related U.S. Application Data
(60) Provisional application No. 63/499,712, filed on May 2, 2023.

(51) Int. Cl. A61K 48/00; A61K 9/00; A61K 38/00; A61P 25/28; C07K 14/47; C12N 15/86

(52) U.S. Cl. CPC A61K 48/0058; A61K 9/0019; A61K 48/0075; A61P 25/28; C07K 14/4702; C12N 15/86; A61K 38/00; C12N 2750/14143; C12N 2830/15; C12N 2830/50

(57) ABSTRACT

Provided are gene therapy vectors, such as adeno-associated virus (AAV), compositions and methods for treatment of diseases caused by misfolded proteins. The disclosed rAAV comprise a nucleotide sequence encoding the BCL2 Associated Athanogene 3 (BAG-3) protein, and methods of administering these rAAV to treat of diseases and disorders associated with protein misfolding and/or aggregation in o a subject in need, which results in increased targeting of aggregation prone proteins for degradation through the BAG3-mediated selective macroautophagy pathway, thereby restoring proteostasis. The disclosed gene therapy vectors, such as rAAV constructs also are used for treatment of inclusion body myositis (IBM) associated with Paget disease of bone and frontotemporal dementia (IBMPFD) and multisystem proteinopathy.

### CROSS REFERENCE TO RELATED APPLICATIONS

This application claims priority benefit of U.S. Provisional Application No. 63/499,712 filed on May 2, 2023, which is incorporated herein by reference in its entirety.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: 58564_SeqListing.xml; Size: 54,030 bytes; Created: Apr. 30, 2024.

### FIELD OF THE INVENTION

The present disclosure provides gene therapy vectors, such as recombinant adeno-associated viral (rAAV) constructs, designed for treatment of diseases and disorders associated with protein misfolding and/or aggregation such as neurodegenerative protein misfolding disorders e.g., amyotrophic lateral sclerosis (ALS) and protein aggregate myopathies (PAM) e.g., limb-girdle muscular dystrophy type 1A (LGMD1A). The present disclosure also provides for gene therapy vectors, such as rAAV constructs, designed for treatment of inclusion body myositis (IBM) associated with Paget disease of bone and frontotemporal dementia (IBMPFD) and/or multisystem proteinopathy. The disclosed rAAV comprise a nucleotide sequence encoding the BCL2 Associated Athanogene 3 (BAG-3) protein, and methods of administering these rAAV to treat of diseases and disorders associated with protein misfolding and/or aggregation in o a subject in need.

### BACKGROUND

Most proteins must fold properly into well-defined, three-dimensional structures to function. Moreover, cells must maintain protein homeostasis (proteostasis), balancing new protein synthesis, folding, transport, and timely degradation of proteins that are no longer needed or that are damaged or misfolded beyond repair or refolding. Cells have therefore evolved a vast, tightly connected proteostasis network (PN), encompassing the translational machinery, molecular chaperones and cochaperones, the ubiquitin-proteasome system (UPS), and the autophagy machinery, to regulate proteostasis. These multiple quality control mechanisms ensure that nascent polypeptides are properly folded, mature proteins maintain their functional conformation, and misfolded proteins are refolded correctly or degraded.

However, despite the presence of numerous and effective control systems, the accumulation of misfolded proteins that cause proteotoxicity and proteostatic collapse, is not uncommon. Factors like mistranslation by defective ribosomes, cellular aging, physiological stressors such as oxidative stress and altered pH, and protein mutations can all shift the equilibrium from the native conformational state to an unfolded or partly folded state leading to misfolding. Compared to the unfolded proteins, the partly folded protein intermediates are more susceptible to degradation by the intracellular quality control systems. However, these folding intermediates are also more prone to self-association forming higher order aggregates due to the presence of large patches of contiguous surface hydrophobicity (Fink A L. 1998. Fold Des. 3 (1): R9-R23). These higher order aggregates are highly resistant to degradation, and they are also prone to mistargeting to the wrong cellular compartment. Protein misfolding or altered native state stability and aggregation is the underlying mechanism of many human disorders collectively termed proteinopathies, which include, but are not limited to, those affecting the nervous system (neuroproteinopathies) e.g., amyotrophic lateral sclerosis (ALS), Alzheimer's disease (AD) and Huntington's disease (HD), striated muscles (protein aggregate myopathies (PAM)) e.g., limb girdle muscular dystrophy type 2Q (LGMD2Q/LGMDR17) and limb-girdle muscular dystrophy type 1A (LGMD1A), and the cardiac system (proteotoxic cardiac diseases e.g., Desmin Related Myopathy (DRM)).

There are no known effective therapies for treating diseases associated with misfolded protein or protein aggregates, and available therapeutics remain palliative. A need therefore exists for therapies that reverse or eliminate protein misfolding and aggregation, and thereby restore proteostasis. Clearance of the misfolded proteins by upregulating autophagy represents a promising therapeutic strategy in these diseases.

### SUMMARY

Provided herein is a polynucleotide sequence comprising a transcriptional control element and a nucleotide sequence encoding the human Bcl-associated athanogene 3 protein (hBAG3). In some embodiments, the nucleotide sequence encoding hBAG3 is at least 90% identical to nucleotides 1112-2839 of SEQ ID NO: 1 or at least 90% identical to nucleotides 981-2708 of SEQ ID NO: 2, which encodes a protein that retains BAG3 activity. In some embodiments, the nucleotide sequence encoding hBAG3 comprises nucleotides 1112-2839 of SEQ ID NO: 1 or nucleotides 981-2708 of SEQ ID NO: 2. In some embodiments, the nucleotide sequence encoding hBAG3 comprises the nucleotide sequence set forth in SEQ ID NO: 8.

In one aspect, the disclosure encompasses the use of a BAG-3 protein to increase the recognition and degradation of misfolded or aggregated proteins or protein inclusions. In certain aspects, recognition and degradation of misfolded or aggregated proteins via a BAG3 protein described herein can treat or prevent a disease or disorder associated with the misfolded or aggregated protein.

For example, in one embodiment, degradation of mutant copper-zinc superoxide dismutase enzyme (SOD1), via BAG3 described herein, can treat or prevent Amyotrophic Lateral Sclerosis (ALS). In another exemplary embodiment, degradation of mutant myotilin, via BAG3 described herein, can treat or prevent can treat or prevent myotilinopathies like limb girdle muscular dystrophy 1A (LGMD1A) and other diseases associated with degradation of misfolded or aggregated proteins.

In one aspect, the disclosure provides compositions and methods for robust and long-term increase in the expression and/or activity of BAG3 in muscles or CNS tissue in the treatment of protein misfolding diseases or disorders. In certain embodiments, the composition comprises a nucleic acid molecule, expression vector, protein, peptide, small molecules, or the like, which increases the expression, activity, or both of a BAG3 protein.

Disclosed herein are methods and compositions for treating or preventing a disease or disorder associated with misfolded proteins or protein aggregates in a mammalian subject. The disclosure describes gene therapy vectors, e.g., AAV, expressing the human Bcl-associated athanogene 3 (BAG3) gene and methods of delivering BAG3 to muscles and the CNS to decreases the accumulation of misfolded or aggregated proteins, and/or prevent or treat a disease or disorder associated with misfolded proteins or protein aggregates. In addition, the disclosure provides combination therapies and approaches using gene therapy vectors to deliver BAG3 to address protein misfolding and aggregation and gene therapy vectors delivering the neurotrophic factor, Neurotrophin-3 (NT-3) to promote nerve regeneration and further improve muscle function.

The present disclosure is based, at least in part, on the discovery that increased expression of BAG3 (e.g., using a vector) in animal models of myofibrillar myopathies (MFM) with protein aggregation, e.g., LGMD1A decreased the accumulation of intracellular inclusions of cytotoxic protein aggregates associated with the disease, decreased neuronal cell death e.g., of motor neurons and improved muscle function. Likewise, in a mouse model of ALS, a neurodegenerative disease associated with protein misfolding, BAG3 gene therapy reduced neuronal cell death, improved muscle function, and increased overall survival, effects that were synergistic when the BAG3 therapy was combined with NT3.

Accordingly, in one aspect, provided herein is a polynucleotide sequence comprising a transcriptional control element and a nucleotide sequence encoding the human Bcl-associated athanogene 3 protein (hBAG3). In some embodiments, the nucleotide sequence encoding hBAG3 is at least at least 90% identical to nucleotides 1112-2839 of SEQ ID NO: 1 or at least 90% identical to nucleotides 981-2708 of SEQ ID NO: 2, which encodes a protein that retains BAG3 activity. In some embodiments, the nucleotide sequence encoding hBAG3 comprises nucleotides 1112-2839 of SEQ ID NO: 1 or nucleotides 981-2708 of SEQ ID NO: 2. In some embodiments, the nucleotide sequence encoding hBAG3 consists of the nucleotide sequence set forth in SEQ ID NO: 8.

Transcriptional control elements include but are not limited to, promoters, enhancers and/or polyadenylation signal sequences. Examples of transcriptional control elements include the cytomegaloviruses (CMV promoter), CMV enhancer, miniCMV promoter, MHCK7, CK8 promoter, the chicken β actin promoter, the P546 promoter simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the elongation factor-1a promoter, the hemoglobin promoter, and the creatine kinase promoter.

In an exemplary embodiment, the transcriptional control element is a muscle specific control element. The term “muscle specific control element” refers to a nucleotide sequence that regulates expression of a coding sequence that is specific for expression in muscle tissue. These control elements include enhancers and promoters.

Exemplary muscle-specific promoters include one or more of a human skeletal actin gene element, a cardiac actin gene element, a desmin promoter, a skeletal alpha-actin (ASKA) promoter, a troponin I (TNNI2) promoter, a myocyte-specific enhancer binding factor MEF binding element, a muscle creatine kinase (MCK) promoter, a truncated MCK (tMCK) promoter, a myosin heavy chain (MHC) promoter, a hybrid a-myosin heavy chain enhancer-/MHC enhancer-promoter (MHCK7) promoter, a C5-12 promoter, a murine creatine kinase enhancer element, a skeletal fast-twitch troponin C gene element, a slow-twitch cardiac troponin c gene element, a slow-twitch troponin I gene element, hypoxia-inducible nuclear factor (HIF)-response element (HRE), a steroid-inducible element, and a glucocorticoid response element (GRE).

The disclosure provides constructs comprising the muscle specific control element truncated MCK (tMCK) promoter. For example, the tMCK promoter nucleotide sequence comprises nucleotides 164-884 of SEQ ID NO: 1. In some embodiments, the tMCK comprises the nucleotide sequence set forth in SEQ ID NO: 11.

In certain embodiments, the transcriptional control element comprises a chicken beta actin (CBA) promoter. For example, the CBA promoter sequence comprises nucleotides 495-749 of SEQ ID NO: 2. In certain embodiments, the transcriptional control element comprises a CMV enhancer. For example, the CMV enhancer sequence comprises nucleotides 209-463 of SEQ ID NO: 2. In certain embodiments, the transcriptional control element is a hybrid CMV enhancer/chicken β-actin (CBA) promoter.

In an exemplary embodiment, the transcriptional control element is a neuronal-specific control element. The term “neuronal-specific control element” refers to a nucleotide sequence that regulates expression of a coding sequence that is specific for expression in neuronal tissue. These control elements include neuronal-specific enhancers and promoters.

Exemplary neuronal-specific promoters include one or more of a platelet-derived growth factor B-chain (PDGFβ) promoter, synapsin-1 (Syn) promoter, synapsin-2 promoter, tyrosine hydroxylase promoter, dopamine β-hydroxylase (DBH) promoter, hypoxanthine-guanine phosphoribosyltransferase (HPRT) promoter, low-affinity nerve growth factor receptor (LNGFR) promoter, Calcitonin Gene-Related Peptide promoter (CGRP promoter), Choline Acetyl Transferase (ChAT) promoter, Neuron Specific Enolase (NSE) promoter, Calcium/Calmodulin Dependent Protein Kinase II (CaMKII) promoter, methyl CpG binding protein 2 (MeCP2) promoter, Glial fibrillary acidic protein (GFAP) promoter, Calbindin 2 promoter, Motor neuron and pancreas homeobox 1 (MNX1) promoter also known as the Hb9 promoter, Nestin promoter, Parvalbumin (PVALB) promoter, and the Somatostation (SST) promoter.

In some embodiments, any of the polynucleotides disclosed herein further comprise a SV40 enhancer and/or an intron, such as a SV40 intron or a chimeric intron. For example, the SV40 intron comprises nucleotides 830-926 of SEQ ID NO: 2. In some embodiments, the chimeric intron comprises nucleotides 937-1069 of SEQ ID NO: 1. In addition, any of the polynucleotides disclosed herein further comprise a polyadenylation signal sequence, which optionally is a synthetic polyadenylation signal sequence. The polynucleotide sequences disclosed herein comprise an inverted terminal repeat (ITR), such as a mutant ITR or a wild type ITR.

The disclosure also provides for a polynucleotide sequence that is an AAV genome. For example, the disclosure provides an AAV genome or polynucleotide sequence comprising a nucleotide sequence that is at least 80%, 81%, 82%, 83%, 84%, 85%, 86%, 87%, 88%, 89%, 90%, 91%, 92%, 93%, 94%, 95%, 96%, 97%, 98% or 99% identical to nucleotides 1 to 3245 of SEQ ID NO: 1. In certain embodiments, the polynucleotide sequence that is an AAV genome comprises nucleotides 1 to 3245 of SEQ ID NO: 1. In some embodiments, the polynucleotide sequence that is an AAV genome comprises the nucleotide sequence set forth in SEQ ID NO: 6. In addition, the rAAV genomes provided herein, hybridizes under stringent conditions to the polynucleotide sequence of nucleotides 1 to 3245 of SEQ ID NO: 1, the nucleotide sequence of SEQ ID NO: 6 or the complement thereof.

The disclosure also provides for a polynucleotide sequence that is an AAV genome. For example, the disclosure provides an AAV genome or polynucleotide sequence comprising a nucleotide sequence that is at least 80%, 81%, 82%, 83%, 84%, 85%, 86%, 87%, 88%, 89%, 90%, 91%, 92%, 93%, 94%, 95%, 96%, 97%, 98% or 99% identical to nucleotides 1 to 3114 of SEQ ID NO: 2. In certain embodiments, the polynucleotide sequence that is an AAV genome comprises nucleotides 1 to 3114 of SEQ ID NO: 2. In some embodiments, the polynucleotide sequence that is an AAV genome comprises the nucleotide sequence set forth in SEQ ID NO: 7. In addition, the rAAV genomes provided herein, hybridizes under stringent conditions to the polynucleotide sequence of nucleotides 1 to 3114 of SEQ ID NO: 2, the nucleotide sequence of SEQ ID NO: 7 or the complement thereof.

The terms “sequence identity”, “percent sequence identity”, or “percent identical” in the context of nucleic acid or amino acid sequences refers to the residues in the two sequences which are the same when aligned for maximum correspondence. The length of sequence identity comparison may be over the full-length of the genome, the full-length of a gene coding sequence, or a fragment of at least about 500 to 5000 nucleotides, is desired. However, identity among smaller fragments, e.g., of at least about nine nucleotides, usually at least about 20 to 24 nucleotides, at least about 28 to 32 nucleotides, at least about 36 or more nucleotides, may also be desired. The percentage identity of the sequences can be determined by techniques known in the art. For example, homology can be determined by a direct comparison of the sequence information between two polypeptide molecules by aligning the sequence information and using readily available computer programs such as ALIGN, ClustalW2 and BLAST. In one embodiment, when BLAST is used as the alignment tool, the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR.

The disclosure also provides for recombinant adeno-associated virus (rAAV) comprising any of the polynucleotide sequences described herein. For example, the rAAV comprises AAV-1, AAV-2, AAV-3, AAV-4, AAV-5, AAV-6, AAV-7, AAV-8, AAV-9, AAV-10, AAV-11, AAV-12, AAV-13, AAV rh.74, AAV rh.10 capsid protein, or a variant thereof. In some embodiments, the rAAV is a AAVrh.74 serotype. In some embodiments, the rAAV is a AAV1 serotype. In some embodiments, the rAAV is a AAV9 serotype.

The disclosure also provides for a recombinant AAV particle comprising any of the polynucleotide sequences disclosed herein or any of the rAAV disclosed herein.

In another embodiment, the disclosure provides for methods of producing a rAAV vector particle comprising culturing a cell that has been transfected with any rAAV vector of the disclosure and recovering rAAV particles from the supernatant of the transfected cells. The disclosure also provides for viral particles comprising any of the recombinant AAV vectors of the disclosure.

The disclosure also provides for compositions comprising any of the rAAV disclosed herein or any of the rAAV particles described herein. In some embodiments, the compositions further comprise a pharmaceutically acceptable carrier. The compositions may also comprise other ingredients such as diluents and adjuvants. Acceptable carriers, diluents and adjuvants are nontoxic to recipients and are preferably inert at the dosages and concentrations employed and include buffers and surfactants such as pluronics.

The disclosure also provides for compositions for treating muscular dystrophy in a subject in need thereof, wherein the composition comprises any of the rAAV disclosed herein or any rAAV particles disclosed herein or any of the compositions disclosed herein.

The disclosure also provides for methods of reducing misfolded proteins or protein aggregates in a subject suffering from a myofibrillar myopathy associated with protein misfolding or aggregation comprising administering any of the rAAV disclosed herein or any rAAV particles disclosed herein or any of the compositions disclosed herein to a subject in need thereof. For example, the subject is suffering from Limb-girdle muscular dystrophy type 1A (LGMD1A) caused by aggregation of mutant myotilin (MYOT).

The disclosure also provides for methods of reducing misfolded proteins or protein aggregates in a subject suffering from a neurodegenerative disease or disorder associated with protein misfolding comprising administering any of the rAAV disclosed herein or any rAAV particles disclosed herein or any of the compositions disclosed herein to a subject in need thereof. For example, the subject is suffering from Amyotrophic lateral sclerosis (ALS) caused by misfolded superoxide dismutase 1 (SOD1).

Exemplary proteins associated with protein misfolding diseases include but are not limited to: desmin, Alpha-crystallin B chain, myotilin, filamin C, BAG family molecular chaperone regulator 3 (BAG-3), Z-band alternatively spliced PDZ-motif containing protein, HSPB8, four-and-a-half LIM domain protein 1, titin, plectin, α-actin or DnaJ heat shock protein family (Hsp40) member B6, alpha-synuclein, amyloid-beta, mutated huntingtin, tau protein, prion proteins, misfolded superoxide dismutase 1 (SOD1), isled amyloid polypeptide (IAPP), Musashi protein, p53, Fused in sarcoma (FUS), Progranulin, TAR DNA-binding protein 43 (TDP-43), misfolded transthyretin protein (TTR), valosin-containing protein (VCP), NOTCH3 receptor, mutated cystatin C, polyglutamine repeats, serum amyloid A (SAA), mutated gelsolin, misfolded rhodopsin, medin, dipeptide repeat protein, or atrial natriuretic peptide.

The disclosure also provides for methods of preventing or treating a disease or disorder associated with misfolded protein or protein aggregates in a subject in need thereof comprising administering any of the rAAV disclosed herein or any rAAV particles disclosed herein or any of the compositions disclosed herein to a subject in need thereof. For example, the subject is suffering from a protein aggregate myopathy (PAM) or a neurodegenerative disease associated with misfolded proteins or protein aggregates.

The disclosure also provides for methods of treating or preventing inclusion body myositis (IBM) or multisystem proteinopathy in a subject in need thereof, the method comprising administering any of the rAAV disclosed herein or any rAAV particles disclosed herein or any of the compositions disclosed herein to a subject in need thereof. For example, the IBM is associated with Paget disease of bone and/or frontotemporal dementia (IBMPFD). In some aspects the subject has a mutation in the valosin-containing protein (VCP) gene. Furthermore, in any of the disclosed methods, the subject is suffering from amyotrophic lateral sclerosis or Charcot-Marie-Tooth type 2.

The disclosure also provides for use of any of the rAAV disclosed herein or any rAAV particles disclosed herein or any of the compositions disclosed herein for the preparation of a medicament for the treatment or prevention of inclusion body myositis (IBM) or multisystem proteinopathy in a subject in need thereof. For example, the IBM is associated with Paget disease of bone and/or frontotemporal dementia (IBMPFD). In some aspects, the subject has a mutation in the valosin-containing protein (VCP) gene. Furthermore, in any of the disclosed uses, the subject is suffering from amyotrophic lateral sclerosis or Charcot-Marie-Tooth type 2.

The disclosure also provides for compositions for the treatment or prevention of inclusion body myositis (IBM) or multisystem proteinopathy in a subject in need thereof, wherein the composition comprises any of the rAAV disclosed herein or any rAAV particles disclosed herein or any of the compositions disclosed herein. For example, the IBM is associated with Paget disease of bone and/or frontotemporal dementia (IBMPFD). In some aspects, the subject has a mutation in the valosin-containing protein (VCP) gene. Furthermore, in any of the disclosed uses, the subject is suffering from amyotrophic lateral sclerosis or Charcot-Marie-Tooth type 2.

Combination therapies are also contemplated. In this regard, any of the foregoing methods described herein may further comprise administering a second nucleic acid encoding a NT-3 polypeptide. In some embodiments, the second nucleic acid encoding a NT-3 polypeptide comprises a nucleotide sequence that is at least 90% identical to the nucleotide sequence of SEQ ID NO: 9. In some embodiments, the second nucleic acid encoding a NT-3 polypeptide comprises the nucleotide sequence set forth in SEQ ID NO: 9. In some embodiments, the second nucleic acid encoding a NT-3 polypeptide comprises a nucleotide sequence encoding an amino acid sequence that is at least 90% identical to SEQ ID NO: 10. In some embodiments, the second nucleic acid encoding a NT-3 polypeptide comprises a nucleotide sequence encoding the amino acid sequence set forth in SEQ ID NO: 10. In some embodiments, the second nucleic acid encoding a NT-3 polypeptide comprises a nucleotide sequence at least 90% identical to nucleotides 1077-1850 of SEQ ID NO: 12. In some embodiments, or 100% identical to nucleotides 1077-1850 of SEQ ID NO: 12.

In some embodiments, the second nucleic acid encoding a NT-3 polypeptide is operably linked to a muscle-specific control element. For example the muscle-specific control element is human skeletal actin gene element, cardiac actin gene element, myocyte-specific enhancer binding factor (MEF) element, muscle creatine kinase (MCK) promoter, tMCK (truncated MCK) promoter, myosin heavy chain (MHC) promoter, MHCK7 promoter (a hybrid version of MHC and MCK), C5-12 (synthetic promoter), murine creatine kinase enhancer element, skeletal fast-twitch troponin C gene element, slow-twitch cardiac troponin C gene element, the slow-twitch troponin I gene element, hypoxia-inducible factor binding element, steroid-inducible element or glucocorticoid response element (GRE). In some embodiments, the second nucleic acid encoding a NT-3 polypeptide is operably linked to a muscle creatine kinase promoter/enhancer sequence. For example, the enhancer/promoter comprises the sequence set out in nucleotides 147-860 of SEQ ID NO: 12.

In some embodiments, a second nucleic acid encoding a NT-3 polypeptide is administered using a second viral vector. The second AAV can be any serotype, for example AAV-1, AAV-2, AAV-3, AAV-4, AAV-5, AAV-6, AAV-7, AAV-8, AAV-9, AAV-10, AAV-11, AAV12, AAV13, Anc80, AAV-B1, AAVrh. 10, AAVrh.74 or variant thereof. In some embodiments, the serotype of the second recombinant AAV capsid is AAV-1. In some embodiments, the second recombinant AAV vector comprises the scAAV1.tMCK.NTF3 rAAV genome that is at least 90% identical to SEQ ID NO: 12, as described in U.S. Patent Publication No. US-2020-0339960 (the disclosure of which is incorporated herein by reference in their entirety). In some embodiments, the scAAV1.tMCK.NTF3 rAAV genome comprised the nucleotide sequence set forth in SEQ ID NO: 12.

In some embodiments, the second nucleic acid encoding a NT-3 polypeptide further comprise an intron such as an SV40-intron or a chimeric intron. In some embodiments, the second nucleic acid encoding a NT-3 polypeptide comprises a chimeric intron sequence set in nucleotides 892-1024 of SEQ ID NO: 12. In addition, the second nucleic acid encoding a NT-3 polypeptide disclosed herein further comprises a polyadenylation signal sequence, which optionally is a synthetic polyadenylation signal sequence. For example, the polyadenylation signal sequence comprises the sequence set out in nucleotides 1860-2059 of SEQ ID NO: 12. The second nucleic acid encoding a NT-3 polypeptide disclosed herein further comprises an inverted terminal repeat (ITR), such as a mutant ITR or a wild type ITR.

In any of the disclosed methods, the rAAV, rAAV particle or the composition is administered using systemic administration, intramuscular injection or intravenous injection. In addition, in any of the method of the invention, the rAAV is administered systemically, such as parental administration by injection, infusion or implantation.

The compositions of the disclosure are formulated for intramuscular injection or intravenous injection. In addition, the compositions of the invention are formulated for systemic administration, such as parental administration by injection, infusion or implantation. In addition, any of the compositions of the disclosure are formulated for administration to a subject suffering from a myofibrillar myopathy associated with protein misfolding or aggregation (such as a Limb-girdle muscular dystrophy type 1A (LGMD1A) or a neurodegenerative disease associated with misfolded proteins or protein aggregates such as ALS), or inclusion body myositis such as IBMPFD or multisystem proteinopathy.

In any of the uses of the disclosure, the medicament is formulated for intramuscular injection or intravenous injection. In addition, in any of the uses of the invention, the medicament is formulated for systemic administration, such as parental administration by injection, infusion or implantation. In addition, any of the medicaments may be prepared for administration in a subject suffering from a myofibrillar myopathy associated with protein misfolding or aggregation (such as a Limb-girdle muscular dystrophy type 1A (LGMD1A) or a neurodegenerative disease associated with misfolded proteins or protein aggregates such as ALS), or inclusion body myositis such as IBMPFD or multisystem proteinopathy.

A “subject,” as used herein, can be any animal, and may also be referred to as the patient. Preferably, the subject is a vertebrate animal, and more preferably the subject is a mammal, such as a domesticated farm animal (e.g., cow, horse, pig) or pet (e.g., dog, cat). in some embodiments, the subject is a human. In some aspects, the subject comprises one or more mutations in the BAG3 gene such as the BAG3 P209L mutation or the GGGGCC hexanucleotide repeat in C9orf72. In some aspects, the subject comprises a mutation in any one of VCP, DES, FLNC, MYOT, CRYAB, ZASP, BAG3, FHL1, TTN, PLEC, ACTA1, HSPB8, SOD1, or DNAJB6 genes.

The foregoing paragraphs are not intended to define every aspect of the disclosure, and additional aspects are described in other sections, such as the Detailed Description. The entire document is intended to be related as a unified disclosure, and it should be understood that all combinations of features described herein are contemplated, even if the combination of features are not found together in the same sentence, or paragraph, or section of this document. The disclosure includes, as an additional aspect, all embodiments of the invention narrower in scope in any way than the variations defined by specific paragraphs above. For example, where certain aspects of the disclosure that are described as a genus, it should be understood that every member of a genus is, individually, an aspect of the disclosure.

### BRIEF DESCRIPTION OF THE DRAWINGS

FIG. 1A provides a schematic of the gene therapy cassette comprising a tMCK enhancer/promoter, the full-length BAG3 cDNA, a SV40 polyA tail, and 5′ and 3′ ITRs.

FIG. 1B provides a schematic of the gene therapy cassette comprising the CBA promoter/CMV enhancer, SV-40 intron, the full-length BAG3 cDNA, a SV40 polyA tail and 5′ and 3′ ITRS.

FIGS. 2A and 2B provide plasmid maps and ORF analysis for ss.pAAV.tMCK.BAG3 and ss.pAAV.CMV.CBA.BAG3 respectively.

FIG. 3 provides a schematic of the full-length NTF3 cDNA cassette with a tMCK enhancer/promoter, an intron to enhance gene expression (I), an ITR and an SV40 polyA tail.

FIG. 4 depicts the presence of 74 kDA hBAG3 protein is seen in the WP-4 lysate (left panel) as well as in the left GAS muscle of TgT57I transgenic (MYOT) mouse (No. 97) and wild type (WT) mouse (No. 98) at 8 weeks post injection with ss.pAAV.CMV.hBAG3 (2.5×10¹¹ vg in 50 μl PBS) (right panel). Un-injected muscles show no hBAG3 bands.

FIGS. 5A and 5B provide representative images of Hematoxylin and eosin (H&E)-stained cross sections of an untreated control right GAS muscle of TgT57I transgenic (MYOT) mouse (A) and a BAG3-treated left GAS muscle (B) at 12 weeks post BAG3 gene therapy via IM injection of ssAAV9.CMV.hBAG3 vector at 2.5×10¹¹ vg showing a significant decrease in the dark purple color intracytoplasmic aggregates compared to untreated control right GAS with greater number of mutant aggregates.

FIG. 5C provides fluorescence micrographs of tissue sections from untreated right GAS muscle control tissue of TgT57I transgenic (MYOT) mouse and AAV-treated left GAS muscle tissue 8 weeks post injection that were fixed and immunostained with an anti-myotilin antibody. The AAV9.BAG3 injected left GAS muscle (left panel) showed fewer myotilin positive areas while the untreated control right GAS muscle (right panel) revealed numerous well delineated aggregates, demonstrating strongly immune positivity for myotilin.

FIG. 5D provides graphs showing quantification of myotilin inclusion density in the immunostained sections provided in FIG. 5C. The left panel provides the quantification by pixel count and the right panel provides mean pixel count quantification. These results demonstrate a significant reduction (p<0.0001) of the pixel counts and mean pixel-count of the treated left GAS muscle (calculated as percent of untreated right GAS in each mouse).

FIG. 6A-6C provide a representative images of H&E-stained sections of tibialis anterior muscles from C57BL/6 wild type (WT) mice 8 weeks post systemic administration of the ssrAAVrh74.tMCK.hBAG3 vector at a dose of 6×10¹² vg. FIGS. 6B and C provide representative images of H&E-stained sections of GAS muscles of male MYOT mice eight weeks following systemic delivery of ssrAAVrh74.tMCK.hBAG3 at 3×10¹² vg (B) or untreated male mice (C). The tissue from the treated mouse showed an overall decrease in particle size compared to tissue from the untreated mouse.

FIGS. 7A-7D provide representative images of H&E-stained sections of GAS and Quad muscles of a male MYOT mouse 8 weeks post systemic delivery of ssrAAVrh74.tMCK.hBAG3 at 3×10¹² vg (A & C respectively) and age and sex matched untreated control muscles (B & D respectively). The treated muscles showed smaller aggregate size, which were less basophilic compared to untreated muscles.

FIGS. 8A-8J provide representative images of H&E-stained sections from right GAS muscles of a male-only cohort of MYOT mice 8 weeks post intramuscular injection with low dose (LD, 3×10¹⁰ vg), intermediate dose (ID, 1×10¹¹ vg), and high dose (HD, 2×10¹¹ vg) ssrAAVrh74.tMCK.hBAG3 vector, the left GAS muscles were untreated controls. Aggregate size was smaller and less basophilic in muscles treated with all three doses.

FIGS. 9-9C provide bar graphs showing myotilin aggregate size distribution of untreated left GAS muscles and right GAS mice muscles treated with LD, ID and HD ssrAAVrh74.tMCK.hBAG3 vector whose H&E images are provided in FIG. 8 (two-way ANOVA, Bonferroni multiple comparison test, ****p<0.0001, ***p<0.001). The treated right muscles showed a significant shift towards smaller size aggregates with all three doses.

FIGS. 10A-10C provides bar graphs showing mean aggregate fluorescent intensity, mean aggregate area (μm²) and % of area occupied by the myotilin aggregates respectively in the LD, ID and HD treated and untreated control muscles (t-test, *p<0.05).

FIGS. 11A-11D provides bar graphs showing significant improvement in rotarod treadmill, grip strength test and maximum tetanic response in MYOT mice 8 months post systemic delivery of AAVrh74.tMCK.hBAG3 at 3×10¹² vg.

FIG. 11E provides a bar graph showing no significant difference in the maximum twitch response 8 months post systemic delivery of AAVrh74.tMCK.hBAG3 at 3×10¹² vg.

FIG. 12A provides fluorescence micrographs of quadriceps muscle sections from untreated (left panel) and BAG3-treated (right panel) MYOT mice, immunostained with myotilin antibody 8 months post injection.

FIG. 12B provides bar graphs showing quantification of myotilin aggregate density (number of aggregates/mm²), which significantly decreased in the treated muscles (t-test, *p<0.05).

FIG. 12C provides bar graphs of myotilin aggregate size distribution/mm² in treated and untreated cohorts (Two-way ANOVA, *p<0.05, ****p<0.0001).

FIG. 13A provides Kaplan-Meier survival curves showing the overall survival of untreated (RL cohort) and SOD1-G93A mice cohorts treated with ss.AAV9.CBA.hBAG3 (4×10¹² vg) and sc.AAV1.tMCK.NT-3 (Neurotrophin-3, 1×10¹¹ vg) as monotherapies or combination therapies, at the preclinical, early, and paralysis stages (40 days, 70 days and 100 days respectively).

FIG. 13B provides bar graphs showing a significant increase in survival for the different treatment cohorts compared to the untreated mice and survival differences amongst the treated cohorts.

FIGS. 14A-14C provide line graphs of Rotarod (A) and grip strength (B) test performances of the mice cohorts described in FIG. 13, over time. Asterisks indicate a significant change (p<0.05) in a treated cohort compared to untreated cohort at given time point (t-test). The untreated cohort showed a steady decline in rotarod, grip strength and electrophysiology tests. For the rotarod test, both BAG3-100, Combo-70, and Combo-40 cohorts showed significant improvements starting one-week post-injection up to 128-135 days old compared to untreated mice. Similar improvement in the grip strength test was observed, predominantly in the Combo-70 and Combo-40 cohorts.

FIG. 15A provides a photomicrograph of a Cresyl violet-stained spinal cord sections displaying the anterior horn area used for neuronal counts.

FIGS. 15B and 15C provide bar graphs showing quantification of large (≥15 μm in diameter) and total neuronal populations in anterior horns of lumbar cord from WT and SOD1.G93A mice at end point and untreated mice at age 70 days (RL-70) respectively.

FIG. 16 provides representative photomicrographs of Cresyl violet-stained paraffin-embedded spinal cord tissue sections showing distribution of anterior horn cells from BAG3-100 (A), RL (B), BAG3-40 (E), NT-3-40 (F), Combo (G) and BAG3-70 (H) SOD1.G93A mice cohorts. The boxed areas in FIGS. 14A and B are shown at higher magnification (X 20 objective) in FIGS. 14C and D respectively.

FIG. 17 provides the sequence of ss.pAAV.tMCK.BAG3 (SEQ ID NO: 1).

FIG. 18 provides the sequence of ss.pAAV.CMV.CBA.BAG3 (SEQ ID NO: 2).

FIGS. 19A-19C provide functional outcome graphs at nine months post hBAG3 gene delivery showing (A) treadmill, (B) rotarod, and (C) grip strength data.

FIGS. 20A-20E provide representative (A) H&E, (B, C) Gomori Trichrome, (D) COX, and (E) SDH stained images showing myopathic changes (basophilic regenerating fibers, increased internal nuclei in A), excessive subsarcolemmal mitochondria accumulation (arrows, C-E) and COX-deficient fibers (D) in the gastrocnemius muscle of 4 months of VCP-A232E mice.

FIGS. 21A and 21B provide representative SDH-stained sections from gastrocnemius muscle of (A) untreated, showing fiber size variability and fibers with altered SDH content (abnormal mitochondria) and (B) BAG3-treated mice at endpoint showing improvements in mitochondria content and distribution following gene therapy.

FIGS. 22A-22D demonstrate the effect of BAG3 gene therapy on gastrocnemius muscle. Graphs showing changes observed in (A) fiber size and (B) percent distribution of different fiber types in treated and untreated cohorts. Histograms show fiber size distribution based on (C) number of fibers and (D) percent of fibers.

FIGS. 23A-23C provide a Western blot (A) and graphs showing the protein levels of (B) p62 and (C) LC3 proteins in the gastrocnemius muscle of VCP-A232E mice.

FIG. 24A-24E provide graphs showing relative expression levels (A) Norad, (B) Pum2, (C) Pgc1α, (D) Cox1, and (E) Cox3 in gastrocnemius muscle of treated and untreated VCP-A232E mice.

### DETAILED DESCRIPTION

The present disclosure demonstrates that upregulation of BCL2 Associated Athanogene 3 (BAG3) protein, a molecular co-chaperone, selectively increased the degradation of misfolded and aggregation prone proteins. As misfolded and aggregation prone proteins play a role in the pathology of a variety of disorders and diseases associated with the accumulation of misfolded proteins and/or aggregates, upregulation of BAG3 protein is an effective therapy for these disorders and diseases.

The disclosed therapies are based on the premise that manipulating autophagy will effectively treat protein aggregate associated disease. The pathogenesis of protein aggregate myopathies (PAM) such as myofibrillar myopathies, or the neurodegenerative phenotype of central nervous system (CNS) disorders associated with aggregate-prone proteins are related to the continuous presence of a mutant protein. Studies have shown that the neurodegenerative symptoms in a mouse model of Huntington disease were alleviated by direct RNA interference targeting the mutant huntingtin¹ or halting the expression of the mutant aggregate-prone protein in symptomatic mice^{2, 3}. A second approach to elevate the toxicity of the aggregate-prone proteins could be removal by enhancing its degradation. A highly conserved BAG family co-chaperone BAG3 (Bcl-2-associated athanogene 3) is uniquely situated in the degradation pathway with capacity to induce autophagy to clear misfolded proteins^{4, 5,6-8}. Under acute stress and during cellular aging, BAG3 in concert with the molecular chaperones HSP70 (heat-shock protein 70) and small heat-shock protein HSPB8 as well as the ubiquitin binding protein or receptor p62/SQSTM1, specifically targets aggregation-prone proteins to autophagic degradation. Thereby, BAG3-mediated selective macroautophagy represents an ultimate adaptive safeguarding and emergency system of the protein quality control, which is activated under pathophysiological conditions to ensure cellular proteostasis. In addition, BAG3-mediated selective macroautophagy is also involved in the clearance of aggregated proteins associated with neurodegenerative disorders and protein aggregate myopathies^{9, 10}.

Another BAG family protein BAG1 with HSP70 and STUB1 (STIP1 Homology And U-Box Containing Protein 1) predominantly mediates the degradation of poly-ubiquitinated proteins by the proteasome under physiological conditions^{11, 12}. BAG3 triggers the turnover of polyubiquitinated proteins by the autophagic-lysosomal system under pathophysiological conditions¹³⁻¹⁵. The BAG1 and BAG3 expression levels are reciprocally regulated during cellular aging and under acute stress. Under physiological conditions, a high BAG1 expression, but a low BAG3 expression could be detected; while under pathophysiological conditions, the BAG3 level is elevated and the BAG1 level is decreased. The BAG1 to BAG3 expression switch is accompanied by a functional switch from HSP70-BAG1-mediated proteasomal degradation to HSP70-BAG3-mediated selective macroautophagy¹⁶. Therefore, the present disclosure provides for BAG3 gene therapy as a treatment option as investigated in rodent models for myodegenerative and neurodegenerative diseases associated with protein aggregates. These results described herein demonstrate for the first time have shown that wild type (WT) BAG3 gene therapy using adeno-associated vectors (AAV) lowered the mutant protein aggregates via BAG3-mediated clearance and these gene therapy vectors are a potential treatment of human disorders resulting from misfolded protein aggregates involving muscle or CNS tissue.

BAG3 and Myopathies with Abnormal Protein Aggregates

### BAG3 in Muscle

BAG3 is highly expressed in muscle and cardiac tissue, co-localizing with Z-disks proteins such as α-actinin and desmin¹⁷. Patients featuring BAG3 mutations are characterized by disrupted Z-disks, the degeneration and disorganization of myofibrils¹⁸. Muscle biopsies from patients with severe early childhood myopathy and restrictive cardiomyopathy resulting from a mutation in BAG3 gene (P209L) showed accumulation of material within abnormal fibers with strong immunoreactivity for BAG3, aB-crystallin, desmin, gelsolin, myotilin, ubiquitin, and dystrophin. In normal muscle, BAG33 was immunolocalized to the Z-disk (colocalizing with myotilin). Studies suggest a mechanism that P209L BAG3 tends to aggregate with the available BAG3 pool leading to BAG3 insufficiency, which results in impaired autophagic activity¹⁹.

### Myofibrillar Myopathies (MFMs)

MFMs are an expanding group of protein aggregate diseases with marked clinical and genetic heterogeneity. This group of disorders are characterized by distinctive histopathology of abnormal protein aggregations and myofibrillar disintegration. All genes causing myofibrillar myopathy encode proteins that either reside in or associate with the Z-disc. In addition to the known disease genes DES, FLNC, MYOT, CRYAB, ZASP, BAG3, FHL1 and TTN, mutations in PLEC, ACTA1, HSPB8 and DNAJB6 have also been associated with proximal MFM clinical phenotype. A subgroup of MFMs, desminopathy, distal myotilinopathy, ZASPopathy and alpha-B crystallin-mutated distal myopathy show similar pathological changes in muscle biopsies, although present with a distal predominant muscle weakness. Common features of distal MFM myopathies are dominant inheritance and adult-onset of symptoms starting in the feet and slowly progressing to encompass other muscle groups. Cardiomyopathy is not a common feature in distal MFM myopathies²⁰.

BAG3 and Neurodegenerative Diseases with Abnormal Protein Aggregates

Neurodegenerative diseases, resulting from different mutant/misfolded protein aggregates, have a common unifying feature, an inability of the protein clearance machinery to efficiently degrade the misfolded proteins and maintain proteostasis. The disposal of aberrant, misfolded proteins is carried out either by the ubiquitin-proteosome machinery or by the autophagic-lysosomal system, although aggregated proteins are primarily degraded by a process termed selective macroautophagy. The selective macroautophagy pathway is mediated by the multifunctional HSP70 co-chaperone BAG3 (BCL-2-associated athanogene 3). Well recognized characteristics of many neurodegenerative disorders involve the aggregation and accumulation of misfolded proteins associated with cytotoxic effects in neurons. Examples of misfolded proteins as hallmark of these disorders include tau protein found in neurofibrillary tangles (in AD), mutant huntingtin with expanded polyQ tract (in HD), mutant androgen receptor with expanded polyQ tract (SBMA) and mutant superoxide dismutase 1 (SOD1, in familial amyotrophic lateral sclerosis, fALS). In this context, BAG3 has been shown to promote the clearance of disease-relevant aggregation-prone proteins including tau⁵, α-synuclein²¹, mutant SOD1⁴, and mutant huntingtin¹³. These observations implicate BAG3 as an important player in maintaining neuronal proteostasis, and therefore therapies that upregulate autophagy can ameliorate neurodegenerative diseases. In various experimental systems, autophagy activation was shown to reduce the accumulation of inclusion bodies and further alleviating neurodegeneration phenotypes (e.g., see data provided in Example 7).

### BAG3 and LGMD1A (Myotilinopathy)

Myotilin is predominantly expressed in skeletal and cardiac muscle, localizes to the sarcomere Z-disc, which is the electron-dense structure that contributes to sarcomere assembly, actin filament stabilization, and muscle force transmission²². At the Z-disc, myotilin interacts with α-actinin-2 (ACTN2)²³ and can bundle actin filaments, suggesting that it has a significant role in anchoring and stabilizing F-actin²⁴. Several myotilin point mutations have been described in patients with limb-girdle muscular dystrophy type 1A (LGMD1A), an autosomal dominant muscle disease with adult onset, characterized by initial weakness of proximal girdle muscles²⁵. Muscle histopathology shows myopathic changes to include extensive autophagic vacuoles and Z-band streaming²⁶. Recent combined proteomic and immunolocalization analyses from patient biopsies provide new insights into the complex regulation of protein degradation in myotilinopathy implying a combination of a toxic gain-of-function leading to myotilin-positive protein aggregates and a loss-of-function caused by a shift in subcellular distribution with a deficiency of myotilin at Z-discs that impairs the integrity of myofibrils²⁷.

### Amyotrophic Lateral Sclerosis (ALS)

ALS is a rapidly progressive neurodegenerative disease caused by the selective loss of upper and lower motor neurons in the brain and spinal cord. Most ALS cases are sporadic, with lack of an apparent genetic linkage, however, 10% of the cases are dominantly inherited familial ALS forms (fALS). The most common genetic forms of ALS result from expanded GGGGCC repeat in noncoding region of C9ORF72. Although the mechanisms by which hexonucleotide repeat expansions (HREs) cause toxicity is not clear, it has been proposed that toxic HNE-gain-of function disease mechanisms (repeat RNA toxicity and dipeptide repeat protein production) work together with C90rf72-mediated autophagy impairment resulting in the aggregation of dipeptide repeat proteins and TAR DNA binding protein, TARBP/TDP-43.

The second most common cause of these familial cases (~20%) is attributed to mutations in a gene encoding for the ubiquitous cytoplasmic Cu/Zn-superoxide dismutase (SOD1)³², which lead to accumulation of misfolded SOD1 proteins and to the death of motor neurons. The accumulation of misfolded proteins in spinal motor neurons is a hallmark of both familial and sporadic ALS. Cellular mechanisms responsible for maintaining protein homeostasis and preventing protein aggregation were compromised not only in familial but also in sporadic ALS patients supported by the presence of ubiquitinated insoluble inclusions in both familial and sporadic ALS cases that are SOD1 immunoreactive.

In one aspect, the present disclosure provides compositions and methods to treat or prevent a disease or disorder associated with misfolded protein or protein aggregates. It is demonstrated herein that BAG3 has a role in targeting misfolded or aggregated proteins for autophagic degradation. Thus, in certain aspects, the compositions and methods of the present disclosure can be used to selectively eliminate intracellular or extracellular misfolded proteins, protein aggregates, or protein inclusions.

For example, in certain embodiments, the disclosure provides compositions and methods to treat or prevent protein aggregate myopathies (PAMs) in a subject in need thereof. PAMs are a group of inherited or acquired muscle disorders characterized morphologically by abnormal accumulation of proteins within muscle fibers, which include but are not limited to myofibrillar myopathies (MFMs), actin-related myopathies (actinopathy) and myosin storage myopathy (MSM) (Olive et al. 2015 Hum Mol Genet. 24 (21): 6264). For example, in certain embodiments, the compositions and methods are used for the treatment or prevention of PAMs associated with misfolded proteins and/or protein aggregates of α-actin (ACTA1), nebulin/nebulette (NEBL), myosins (MYOs), titin (TTN), filamin C (FLNC), myotilin (MYOT), Z-band alternatively spliced PDZ-motif containing protein (ZASP), four-and-a-half LIM domain protein 1 (FHL1), desmin (DES), plectin (PLEC), valosin-containing protein (VCP), DnaJ heat shock protein family (Hsp40) member B6 (DNAJB6), BAG family molecular chaperone regulator 3 (BAG-3), αB-crystallin, synemin, Alpha-crystallin B chain (CRYAB), Bifunctional UDP-N-acetylglucosamine 2-epimerase/N-acetylmannosamine kinase (GNE), selenoprotein N (SELENON), LIM domain-binding protein (LDBs), synemin (SYNM), and Poly(A) Binding Protein Nuclear 1 (PABPN1).

For example, in certain embodiments, the disclosure provides compositions and methods for the treatment or prevention of myofibrillar myopathies (MFMs) in a subject in need thereof. Exemplary MFMs include but are not limited to Desminopathy (DES gene), Alpha-B crystallinopathy (CRYAB gene), Myotilinopathy (MYOT gene), a Filaminopathy (FLNC gene), BAG3-related myofibrillar myopathy (BAG3 gene), a ZASPopathy (ZASP gene) or HSPB8 myopathy (HSPB8 gene).

In some embodiments, the disclosure encompasses the treatment or prevention of neuromuscular conditions having diseases causing mutations that display features of myofibrillar myopathy. Exemplary neuromuscular conditions with MFM features include, but are not limited to, reducing body myopathy (FHL1 gene), hereditary myopathy with early respiratory failure (HMERF gene), epidermolysis bullosa simplex with muscular dystrophy (PLEC gene), MFM-actinopathy (ACTA1 gene) and limb girdle muscular dystrophy type 1D (DNAJB6 gene).

In some embodiments, the disclosure provides compositions and methods for the treatment of neurodegenerative diseases or disorders associated with misfolded proteins or protein aggregates. For example, in certain embodiments, the compositions and methods are used for the treatment or prevention of diseases and disorders associated with misfolded proteins and/or protein aggregates of amyloid-beta, alpha-synuclein, tau, prions, SOD1, TDP-43, FUS, p53, p53 mutants, or proteins associated with polyglutamine repeats, such as huntingtin and ataxins.

Exemplary neurodegenerative diseases associated with misfolded proteins or protein aggregates include, but are not limited to, amyotrophic lateral sclerosis (ALS), transmissible spongiform encephalopathies (prion disease), synucleinopathies, dementia with Lewy bodies (DLB), multiple system atrophy (MSA), tauopathies, Frontotemporal lobar degeneration (FTLD), Frontotemporal dementia (FTD), Sporadic or familial with or without motor-neuron disease (MND), corticobasal degeneration, frontotemporal lobar degeneration with ubiquitin-positive inclusions, Argyrophilic grain disease, Pick's disease, Amyotrophic lateral sclerosis (ALS), Sporadic ALS, Alzheimer's disease (AD, sporadic and familial), Down syndrome, Familial British dementia, Polyglutamine (polyQ) diseases (Huntington's disease (HD), dentatorubral-pallidoluysian atrophy (DRPLA), spinal bulbar muscular atrophy (SBMA), and six spinocerebellar ataxias (SCA1, SCA2, SCA3, SCA6, SCA7, and SCA17)), Hippocampal sclerosis dementia or Parkinson's disease (PD).

However, the present invention is not limited to the treatment or prevention of neurodegenerative disorders and myofibrillar myopathies. Rather, the disclosure encompasses the treatment or prevention of any disease or disorder associated with a misfolded protein or protein aggregate. Other such diseases and disorders include, but are not limited to AL amyloidosis, AA amyloidosis, Familial Mediterranean fever, senile systemic amyloidosis, familial amyloidotic polyneuropathy, hemodialysis-related amyloidosis, ApoAI amyloidosis, ApoAII amyloidosis, ApoAIV amyloidosis, Finnish hereditary amyloidosis, lysozyme amyloidosis, fibrinogen amyloidosis, Icelandic hereditary cerebral amyloid angiopathy, type II diabetes, medullary carcinoma of the thyroid, atrial amyloidosis, hereditary cerebral hemorrhage with amyloidosis, pituitary prolactinoma, injection-localized amyloidosis, aortic medial amyloidosis, hereditary lattice corneal dystrophy, corneal amyloidosis associated with trichiasis, cataract, calcifying epithelial odontogenic tumor, pulmonary alveolar proteinosis, inclusion-body myostis, and cuteaneous lichen amyloidosis.

In one aspect the disclosure encompasses the use of a BAG-3 protein to increase the recognition and degradation of misfolded or aggregated proteins or protein inclusions. In certain aspects, recognition and degradation of misfolded or aggregated proteins via a BAG3 protein described herein can treat or prevent a disease or disorder associated with the misfolded or aggregated protein.

For example, in one embodiment, degradation of mutant copper-zinc superoxide dismutase enzyme (SOD1), via BAG3 described herein, can treat or prevent Amyotrophic Lateral Sclerosis (ALS). In another exemplary embodiment, degradation of mutant myotilin, via BAG3 described herein, can treat or prevent can treat or prevent myotilinopathies like limb girdle muscular dystrophy 1A (LGMD1A) and other diseases associated with degradation of misfolded or aggregated proteins.

In one aspect, the disclosure provides compositions and methods for robust and long-term increase in the expression and/or activity of BAG3 in muscles or CNS tissue in the treatment of protein misfolding diseases or disorders. In certain embodiments, the composition comprises a nucleic acid molecule, expression vector, protein, peptide, small molecules, or the like, which increases the expression, activity, or both of a BAG3 protein.

### BAG3 and Inclusion Body Myositis and Multisystem Proteinopathy

Inclusion body myositis (IBM) associated with Paget disease of bone and frontotemporal dementia (IBMPFD), considered one of the hereditary forms of IBM, is an adult-onset progressive dominant disorder with proximal and distal muscle weakness and mild to severe myopathy. The myopathy is present in 90% of cases, Paget's disease 42%, and 30% have frontotemporal dementia⁴⁵. The myopathy begins at an average age of 43 years, similar to the bone disease, while the dementia usually starts about a decade later⁴⁵. IBMPFD is caused by mutations in the valosin-containing protein (VCP) gene. There is no correlation between mutation type and incidence of clinical features associated with the valosin-containing protein (VCP) gene. The spectrum of other diseases associated with mutations in VCP include amyotrophic lateral sclerosis⁴⁶² and Charcot-Marie-Tooth type 2⁴⁷, accounting for what has become a preferred name for this condition: “Multisystem proteinopathy”^{47, 48}. Serum CK may be normal but is variable and commonly elevated in IBMPFD. The VCP gene has 17 exons with mutations reported in 11 exons. The mutations associated with IBMPFD and/or familial ALS are all exonic missense mutations. A232E mutation is associated with a more severe clinical phenotype characterized by earlier onset and more aggressive myopathy compared to the cases with other VCP mutations⁴⁹.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, certain materials and methods are described herein. In describing and claiming the present invention, the following terminology will be used.

As used herein “BAG3”, “BAG3 molecules”, “BCL2-associated athanogene 3 (BAG3) genes”, “BCL2-associated athanogene 3 (BAG3) molecules” are inclusive of all family members, mutants, cDNA sequences, alleles, fragments, species, coding and noncoding sequences, sense and antisense polynucleotide strands, etc. (HGNC (939) Entrez Gene (9531) Ensembl (ENSG00000151929) OMIM (603883) UniProtKB (095817)). For example, the BAG3 gene or cDNA nucleotide sequence is set out as SEQ ID NO: 3, SEQ ID NO: 4, or SEQ ID NO: 8 or the sequence of nucleotides 1112-2839 of SEQ ID NO 1 or nucleotides 981-2708 of SEQ ID NO: 2. Similarly, “BAG3”, “BAG3 molecules”, “BCL2-associated athanogene 3 (BAG3) proteins” also refer to BAG3 polypeptides or fragment thereof, proteins, variants, derivatives etc. For example, the BAG3 protein comprises amino acid sequence of SEQ ID NO: 5. The term “molecule,” thus encompasses both the nucleic acid sequences and amino acid sequences of BAG3.

As used herein, the term “AAV” is a standard abbreviation for adeno-associated virus. Adeno-associated virus is a single-stranded DNA parvovirus that grows only in cells in which certain functions are provided by a co-infecting helper virus. There are currently thirteen serotypes of AAV that have been characterized. General information and reviews of AAV can be found in, for example, Carter, 1989, Handbook of Parvoviruses, Vol. 1, pp. 169-228, and Berns, 1990, Virology, pp. 1743-1764, Raven Press, (New York). However, it is fully expected that these same principles will be applicable to additional AAV serotypes since it is well known that the various serotypes are quite closely related, both structurally and functionally, even at the genetic level. (See, for example, Blacklowe, 1988, pp. 165-174 of Parvoviruses and Human Disease, J. R. Pattison, ed.; and Rose, Comprehensive Virology 3:1-61 (1974)). For example, all AAV serotypes apparently exhibit very similar replication properties mediated by homologous rep genes; and all bear three related capsid proteins such as those expressed in AAV2. The degree of relatedness is further suggested by heteroduplex analysis which reveals extensive cross-hybridization between serotypes along the length of the genome; and the presence of analogous self-annealing segments at the termini that correspond to “inverted terminal repeat sequences” (ITRs). The similar infectivity patterns also suggest that the replication functions in each serotype are under similar regulatory control.

A recombinant “vector” or “AAV vector” as used herein refers to a vector that is derived from the wild type genome of a virus, such as AAV by using molecular methods to remove the wild type genome from the virus (e.g., AAV), and replacing with a non-native nucleic acid, such as a heterologous polynucleotide sequence of interest or transgenes (e.g., a therapeutic gene expression cassette). Typically, for AAV one or both inverted terminal repeat (ITR) sequences of the wild type AAV genome are retained in the AAV vector where they flank the inserted transgene. A recombinant viral vector is distinguished from a viral genome, since all or a part of the viral genome has been replaced with a non-native sequence with respect to the viral genomic nucleic acid such as a heterologous polynucleotide sequence. Incorporation of the non-native sequence therefore defines the viral vector (e.g., AAV) as a “recombinant” vector, which in the case of AAV can be referred to as an “rAAV vector.” Such AAV vectors can be replicated and packaged into infectious viral particles when present in a host cell that has been transfected with a vector encoding and expressing rep and cap gene products.

As used herein, the term “self-complementary AAV vector” (scAAV) refers to a vector containing a double-stranded vector genome generated by the deletion of the terminal resolution site (dTR) from one of the ITRs of the AAV. The absence of a TR prevents the initiation of replication at the vector terminus where the TR is not present. In general, scAAV vectors generate single-stranded, inverted repeat genomes, with a wild-type (wt) AAV TR at each end and a mutated TR (mTR) in the middle.

An “AAV virion” or “AAV viral particle” or “AAV vector particle” refers to a viral particle composed of at least one AAV capsid protein and an encapsidated polynucleotide AAV vector. If the particle comprises a heterologous polynucleotide (i.e., a polynucleotide other than a wild-type AAV genome such as a transgene to be delivered to a mammalian cell), it is typically referred to as an “AAV vector particle” or simply an “AAV vector”. Thus, production of AAV vector particle necessarily includes production of AAV vector, as such a vector is contained within an AAV vector particle.

As used herein, the term “operably linked” refers to a to a juxtaposition of polynucleotide (or polypeptide) elements, wherein the elements are in a functional relationship permitting them to operate in the expected manner. For instance, a promoter is operably linked to a coding sequence if the promoter helps initiate transcription of the coding sequence. There may be intervening residues between the promoter and coding region so long as this functional relationship is maintained. Generally, but not always, operably linked means that the nucleic acid sequences being linked are contiguous and, where necessary to join two protein coding regions, contiguous and in the same reading frame.

“Transcriptional control element” refers to an element or the nucleic acid sequence of an element that regulates the expression of a nucleotide sequence to which it is operably linked. A transcriptional control element is “operably linked” to a nucleotide sequence when the transcriptional control element controls and regulates the transcription and/or the translation of the nucleotide sequence. Thus, an transcriptional control element can include promoters, enhancers, internal ribosome entry sites (IRES), transcription terminators, a start codon in front of a protein-encoding gene, splicing signal for introns, and stop codons. The term “transcriptional control element” is intended to include, at a minimum, an element or the sequence of an element whose presence is designed to influence expression and can also include additional advantageous components. The term can also include the design of a nucleic acid sequence such that undesirable, potential initiation codons in and out of frame, are removed from the sequence. It can also include the design of a nucleic acid sequence such that undesirable potential splice sites are removed. It includes sequences or polyadenylation sequences (PA) which direct the addition of a polyA tail, i.e., a string of adenine residues at the 3′-end of a mRNA, sequences referred to as polyA sequences. It also can be designed to enhance mRNA stability. Transcriptional control elements which affect the transcription and translation stability, e.g., promoters, as well as sequences which effect the translation, e.g., Kozak sequences, are known in the art. Transcriptional control elements can be of such nature as to modulate the nucleotide sequence to which it is operably linked such that lower expression levels or higher expression levels are achieved.

As used herein, the term “promoter” refers to a nucleotide sequence, usually upstream (5′) to its coding sequence, that controls the expression of the coding sequence by providing the recognition for RNA polymerase and other factors required for proper transcription. “Promoter” includes a minimal promoter that is a short DNA sequence comprised of a TATA-box and other sequences that serve to specify the site of transcription initiation, to which regulatory elements are added for control of expression. “Promoter” also refers to a nucleotide sequence that includes a minimal promoter plus regulatory elements that is capable of controlling the expression of a coding sequence or functional RNA. This type of promoter sequence consists of proximal and more distal upstream elements, the latter elements often referred to as enhancers. Accordingly, an “enhancer” is a DNA sequence that can stimulate promoter activity and may be an innate element of the promoter or a heterologous element inserted to enhance the level or tissue specificity of a promoter. It is capable of operating in both orientations (normal or flipped) and is capable of functioning even when moved either upstream or downstream from the promoter. Both enhancers and other upstream promoter elements bind sequence-specific DNA-binding proteins that mediate their effects. Promoters may be derived in their entirety from a native gene or be composed of different elements derived from different promoters found in nature, or even be comprised of synthetic DNA segments. A promoter may also contain DNA sequences that are involved in the binding of protein factors that control the effectiveness of transcription initiation in response to physiological or developmental conditions. A “constitutive” promoter is a promoter that is active in most tissues under most physiological and developmental conditions. An “inducible” promoter is a promoter that is physiologically or developmentally regulated, e.g., by the application of a chemical inducer. A “tissue specific” promoter is only active in specific types of tissues or cells.

As used herein, the term “nucleic acid construct” or “expression construct” or “expression cassette” is intended to mean a nucleic acid molecule (typically comprised of DNA) capable of directing expression of a particular nucleotide sequence in an appropriate host cell, comprising a promoter operably linked to a nucleotide sequence of interest that is operably linked to termination signals. It also typically comprises sequences required for proper translation of the nucleotide sequence. The coding region usually codes for a protein of interest but may also code for a functional RNA of interest, for example antisense RNA or a nontranslated RNA, in the sense or antisense direction. The expression cassette comprising the nucleotide sequence of interest may be chimeric, meaning that at least one of its components is heterologous with respect to at least one of its other components. The expression cassette may also be one that is naturally occurring but has been obtained in a recombinant form useful for heterologous expression. Such expression cassettes will comprise the transcriptional initiation region linked to a nucleotide sequence of interest. Such an expression cassette may be provided with a plurality of restriction sites for insertion of the gene of interest to be under the transcriptional regulation of the regulatory regions. The expression cassette may additionally contain selectable marker genes.

The term “intron” as used herein refers to a sequence encoded in a DNA sequence that is transcribed into an RNA molecule by RNA polymerase but is removed by splicing to form the mature messenger RNA. A “synthetic intron” refers to a sequence that is not initially replicated from a naturally occurring intron sequence and generally will not have a naturally occurring sequence but will be removed from an RNA transcript during normal post-transcriptional processing. Such synthetic introns can be designed to have a variety of different characteristics, in particular such introns can be designed to have a desired strength of splice site and a desired length. In a preferred embodiment of the present invention, both the molecular switch expression cassette and the therapeutic gene expression cassette include a synthetic intron. The synthetic intron includes consensus sequences for the 5′ splice site, 3′ splice site, and branch point. When incorporated into eukaryotic vectors designed to express therapeutic genes, the synthetic intron will direct the splicing of RNA transcripts in a highly efficient and accurate manner, thereby minimizing cryptic splicing and maximizing production of the desired gene product.

As used herein, the terms “treatment,” “treating,” (and grammatical variations thereof), refer to obtaining a desired pharmacologic and/or physiologic effect including reversing, alleviating, inhibiting the progress of, or preventing a disease, disorder or condition to which such term applies, or one or more symptoms of such disease, disorder or condition.

The terms “individual,” “subject,” and “patient” are used interchangeably herein, and refer to a mammal, including, but not limited to, human and non-human primates, including simians and humans; mammalian sport animals (e.g., horses); mammalian farm animals (e.g., sheep, goats, etc.); mammalian pets (dogs, cats, etc.); and rodents (e.g., mice, rats, etc.). In any of the methods of treatment described herein, the subject may comprise a mutation related to a disease or disorder associated with misfolded protein or protein aggregates. Exemplary mutations include the BAG3 P209L mutation or the GGGGCC hexanucleotide repeat in C9orf72. In some aspects, the subject comprises a mutation in any one of DES, FLNC, MYOT, CRYAB, ZASP, BAG3, FHL1, TTN, PLEC, ACTA1, HSPB8, SOD1, or DNAJB6 genes.

The phrase a “unit dosage form,” as used herein refers to a physically discrete unit of inventive formulation appropriate for the subject to be treated; each unit containing a predetermined quantity optionally in association with a pharmaceutical carrier (excipient, diluent, vehicle or filling agent) which, when administered in one or more doses, produces a desired effect (e.g., prophylactic or therapeutic effect). It will be understood, however, that the total daily usage of the compositions of the present disclosure will be decided by the attending physician within the scope of sound medical judgment. The specific effective dose level for any particular subject will depend upon a variety of factors including the disorder being treated and the severity of the disorder; specific composition employed; age, body weight, general health, sex and diet of the subject; time of administration, duration of the treatment; drugs and/or additional therapies used in combination or coincidental with the inventive compositions, and like factors well known in the medical arts. In some embodiments, unit dosage forms may be within, for example, ampules and vials, including a liquid composition, or a composition in a freeze-dried or lyophilized state; a sterile liquid carrier, for example, can be added prior to administration or delivery in vivo. Individual unit dosage forms can be included in multi-dose kits or containers. AAV vectors or AAV virions, and pharmaceutical compositions thereof can be packaged in single or multiple unit dosage form for ease of administration and uniformity of dosage.

A “therapeutically effective amount” will fall in a relatively broad range determinable through experimentation and/or clinical trials. For example, for in vivo injection, e.g., injection directly into the tissue of a subject (for example, muscle tissue), a therapeutically effective dose will be on the order of from about 10⁶ to about 10¹⁵ of the AAV virions per kilogram bodyweight of the subject. In some embodiments, a therapeutically effective dose will be on the order of from about 10⁸ to 10¹² AAV virions per kilogram bodyweight of the subject. Other effective dosages can be readily established by one of ordinary skill in the art through routine trials establishing dose response curves.

As used herein, “an effective amount” refers to an amount of a compound, agent, substance, formulation or composition that is of sufficient quantity to result in a decrease in severity of disease symptoms, an increase in frequency and duration of disease symptom-free periods, or a prevention of impairment or disability due to the disease affliction. The amount may be as a single dose or according to a multiple dose regimen, alone or in combination with other compounds, agents or substances. One of ordinary skill in the art would be able to determine such amounts based on such factors as a subject's size, the severity of a subject's symptoms, and the particular composition or route of administration selected.

The term “about” or “approximately” as used herein when referring to a measurable value such as an amount of the length of a polynucleotide or polypeptide sequence, dose, time, temperature, and the like, is meant to encompass variations of ±20%, ±10%, ±5%, ±1%, ±0.5%, or even ±0.1% from the specified value, as such variations are appropriate to perform the disclosed methods.

Also as used herein, “and/or” refers to and encompasses any and all possible combinations of one or more of the associated listed items, as well as the lack of combinations when interpreted in the alternative (“or”).

The disclosure provides BCL2 Associated Athanogene 3 (BAG3) gene therapy as a feasible therapeutic strategy to treat human disorders resulting from accumulation of misfolded protein aggregates (proteinopathies). Proteinopathies include, but are not limited to, neurodegenerative diseases and disorders e.g., amyotrophic lateral sclerosis (ALS), Alzheimer's disease (AD) and Huntington's disease (HD) and neuromuscular diseases and disorders also known as protein aggregate myopathies (PAM) e.g., limb girdle muscular dystrophy type 2Q (LGMD2Q/LGMDR17) and limb-girdle muscular dystrophy type 1A (LGMD1A). The disclosed compositions and methods enable robust and long-term expression of the BAG-3 gene in muscles and the central nervous system (CNS) in the treatment of protein misfolding diseases or disorders, wherein BAG3 selectively increases autophagic flux and clearance of the misfolded and aggregation prone proteins. For example, the disclosed compositions and methods increase the recognition and elimination of misfolded proteins. The present invention can thus be used to treat or prevent misfolded proteins, protein aggregates, or protein inclusions, both intracellularly and extracellularly. The present disclosure is related to the discovery of the role of BAG3 as a molecular co-chaperone, for refolding or degrading misfolded or aggregation prone proteins, which play a role in the pathology of a variety of muscular and neurodegenerative disorders.

### Polypeptides and Nucleic Acids Encoding Polypeptides

In certain aspects, the disclosure relates to a polypeptide comprising BAG3 or functional isoforms of BAG3 or a fragment thereof.

The human BAG3 gene, located on the long arm of chromosome 10, encodes a ubiquitously expressed 575 amino acid multi-functional protein that is predominantly expressed in skeletal and cardiac muscles, and in the central nervous system (CNS). The NCBI reference nucleic acid sequence for BAG3 can be found at Genbank under accession number NM_004281.4, which is referred herein as SEQ ID NO: 8. The NCBI reference amino acid sequence for BAG3 can be found at Genbank under accession number NP_004272.2, which is referred herein as SEQ ID NO: 5.

During acute proteotoxic stress, BAG3 mediates a noncanonical macroautophagic pathway termed chaperone-assisted selective autophagy (CASA) in which BAG3 acting as a scaffolding protein interacts and cooperates with the chaperones HSP70 (heat shock protein 70) and HSPB8 (Heat shock protein beta-8) as well as the classical selective autophagy receptor, p62/SQSTM1, to selectively identify and refold or target misfolded and aggregation prone proteins for autophagic-lysosomal degradation (Arndt et al. 2010. Curr. Biol. 20, 143-148). Thus, the BAG3-triggered selective macroautophagic pathway is a crucial cellular safeguard of protein quality control in response to stress including under pathophysiological conditions. This pathway significantly contributes to protein homeostasis in post-mitotic cells, such as differentiated neurons and striated muscle cells. Consequently, BAG3 dysfunction and/or mutations that confer conformational instability to aggregation prone proteins cause a broad range of diseases including skeletal and cardiac myopathies and numerous neurodegenerative diseases.

Collectively, these diseases affect millions of lives around the world and have devastating economic implications. However, despite the attention of the scientific community, these disorders are far from resolved as treatments remain palliative. The disclosure focuses on providing a BAG3 transgene for upregulation of BAG3 protein as a treatment option for skeletal myopathies and neurodegenerative diseases associated with protein aggregates by activating BAG3-mediated selective autophagy.

To accomplish this, a polynucleotide sequence comprising a transcriptional control element; and a nucleotide sequence encoding the human Bcl-associated athanogene 3 protein provided. In certain embodiments, the nucleotide sequence encoding a BAG3 polypeptide encodes the amino acid sequence of SEQ ID NO: 5.

In various aspects, the BAG3 polypeptide comprises an amino acid sequence that has at least 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, or 70% identity to the amino acid sequence set forth in SEQ ID NOs: 5 or the amino acid sequence encoded by nucleotides 1112 to 2839 of SEQ ID NO: 1, nucleotides 981 to 2708 of SEQ ID NO: 2 or the nucleotide sequence of SEQ ID NO: 8.

An amino acid sequence alteration can be, for example, a substitution, a deletion, or an insertion of one or more amino acids, preferably conservative substitutions. A BAG3 polypeptide can have any combination of amino acid substitutions, deletions or insertions where activity of the polypeptide is retained. In one aspect, a BAG3 polypeptide can have a number of amino acid alterations such that its amino acid sequence shares at least 60, 70, 80, 85, 90, 95, 97, 98, 99 or 99.5% identity with the amino acid sequence (SEQ ID NO: 5) encoded by BAG3 cDNA set out as nucleotides 1112 to 2839 of SEQ ID NO: 1, nucleotides 981 to 2708 of SEQ ID NO: 2 or the nucleotide sequence of SEQ ID NO: 8.

### Nucleic Acids Encoding Polypeptides

In certain aspects, the invention relates to isolated and/or recombinant nucleic acids encoding a BAG3 polypeptide. The subject nucleic acids may be single-stranded or double-stranded, DNA or RNA molecules. These nucleic acids are useful as therapeutic agents. For example, these nucleic acids are useful in making recombinant polypeptides which are administered to a cell or a subject as therapeutics. Alternatively, these nucleic acids can be directly administered to a cell or an individual as therapeutics such as in gene therapy.

In some aspects, the BAG3 nucleic acid comprises the nucleotide sequence comprising nucleotides 1112 to 2839 of SEQ ID NO: 1, nucleotides 981 to 2708 of SEQ ID NO: 2 or the nucleotide sequence of SEQ ID NO: 8. In various aspects, the BAG3 nucleic acid comprises a nucleotide sequence that has 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, or 70% identity to the nucleotide sequence comprising nucleotides 1112 to 2839 of SEQ ID NO: 1, nucleotides 981 to 2708 of SEQ ID NO: 2 or the nucleotide sequence of SEQ ID NO: 8. One of ordinary skill in the art will appreciate that nucleic acid sequences complementary to the subject nucleic acids, and variants of the subject nucleic acids are also within the scope of this disclosure. In further embodiments, the nucleic acid sequences of the invention can be isolated, recombinant, and/or fused with a heterologous nucleotide sequence, or in a DNA library.

In other embodiments, nucleic acids of the invention also include nucleotide sequences that hybridize under highly stringent conditions to the nucleotide sequence comprising nucleotides 1112 to 2839 of SEQ ID NO: 1, nucleotides 981 to 2708 of SEQ ID NO: 2 or the nucleotide sequence of SEQ ID NO: 8 or complement sequences thereof. As discussed above, one of ordinary skill in the art will understand readily that appropriate stringency conditions which promote DNA hybridization can be varied. One of ordinary skill in the art will understand readily that appropriate stringency conditions which promote DNA hybridization can be varied. For example, one could perform the hybridization at 6.0× sodium chloride/sodium citrate (SSC) at about 45° C., followed by a wash of 2.0×SSC at 50° C. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or temperature or salt concentration may be held constant while the other variable is changed. In one embodiment, the invention provides nucleic acids which hybridize under low stringency conditions of 6×SSC at room temperature followed by a wash at 2×SSC at room temperature.

Isolated nucleic acids which differ from the subject nucleic acids due to degeneracy in the genetic code are also within the scope of the invention. For example, a number of amino acids are designated by more than one triplet. Codons that specify the same amino acid, or synonyms (for example, CAU and CAC are synonyms for histidine) may result in “silent” mutations which do not affect the amino acid sequence of the protein. However, it is expected that DNA sequence polymorphisms that do lead to changes in the amino acid sequences of the subject proteins will exist among mammalian cells. One skilled in the art will appreciate that these variations in one or more nucleotides (up to about 3-5% of the nucleotides) of the nucleic acids encoding a particular protein may exist among individuals of a given species due to natural allelic variation. Any and all such nucleotide variations and resulting amino acid polymorphisms are within the scope of this disclosure.

In some aspects, the polynucleotide sequence is operatively linked to one or more transcriptional control elements in an expression construct (including, but not limited to, promoters, enhancers or activator sequences, polyadenylation sequences, leader or signal sequences, ribosomal binding sites, transcriptional start and termination sequences, and, translational start and termination sequences) that are functional in target cells. Constitutive or inducible promoters as known in the art are contemplated by the disclosure. The promoters may be either naturally occurring promoters, or hybrid promoters that combine elements of more than one promoter. An expression construct may be present in a cell on an episome, such as a plasmid, or the expression construct may be inserted in a chromosome. In some embodiments, the expression vector contains a selectable marker gene to allow the selection of transformed host cells. Selectable marker genes are well known in the art and will vary with the host cell used.

In certain aspects of the disclosure, the subject nucleic acid is provided in an expression vector comprising a nucleotide sequence encoding a BAG3 polypeptide and operably linked to at least one transcriptional control element. Transcriptional control elements are art-recognized and are selected to direct expression of the polypeptide. Accordingly, the term transcriptional control element includes promoters, enhancers, and other expression control elements. Exemplary transcriptional control element are described in Goeddel; Gene Expression Technology: Methods in Enzymology, Academic Press, San Diego, Calif. (1990). For instance, any of a wide variety of transcriptional control element that control the expression of a DNA sequence when operatively linked to it may be used in these vectors to express DNA sequences encoding a polypeptide. Such useful transcriptional control element, include, for example, an H1 promoter, an EF1-alpha promoter, a minimal EF1-alpha promoter, an unc45b promoter, a CK1 promoter, a CK6 promoter, a CK7 promoter, a CK8e promoter, a cardiac troponin C (cTnC) promoter, a miniCMV promoter, a CMV promoter, a cytomegalovirus/chicken beta-actin promoter (CBA, also known as the CAG promoter), a short CMV early enhancer/chicken β-actin/short β-globin intron (sCAG), a muscle creatine kinase (MCK) promoter, a hybrid alpha-myosin heavy chain enhancer-/MCK enhancer-promoter (MHCK7), a truncated MCK (tMCK) promoter, a minimal MCK promoter, a desmin promoter, the P546 promoter, the simian virus 40 (SV40) early promoter, a mouse mammary tumor virus (MMTV) promoter, a human immunodeficiency virus (HIV) long terminal repeat (LTR) promoter, a MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the elongation factor-1a promoter, the hemoglobin promoter, and the creatine kinase promoter, murine creatine kinase enhancer element, skeletal fast-twitch troponin C gene element, slow-twitch cardiac troponin C gene element, the slow-twitch troponin I gene element, hypoxia-inducible nuclear factor response element, steroid-inducible element or glucocorticoid response element (GRE), the woodchuck hepatitis virus post-transcriptional regulatory element (WPRE), the bovine growth hormone polyadenylation sequence and other elements known to control the expression of genes of prokaryotic or eukaryotic cells or their viruses, and various combinations thereof.

Inducible promoters are also included. Non-limiting examples of inducible promoters include, but are not limited to a metallothionine promoter, a glucocorticoid promoter, a progesterone promoter, and a tetracycline-regulated promoter.

The expression cassette comprising the BAG3 transgene, in some aspects, also includes intron sequences. As is generally known in the art, introns are DNA polynucleotides that are transcribed into RNA and removed during mRNA processing through intron splicing. Polynucleotide cassettes containing introns generally have higher expression than those without introns, i.e., Intron-Mediated Enhancement (IME). Introns can stimulate expression between 2- and 500-fold (Buchman and Berg, 1988. Mol Cel Bio, 8 (10): 4395). Efficiently spliced introns contain a pre-splice donor, branchpoint, and Py rich region (Senapathy et al, 1990; Meth. Enzymol. 183, 252-78; Wu and Krainer, 1999; Mol Cell Biol 19 (5): 3225-36). 5′ introns are generally more efficient compared to introns at the 3′ end (Huang and Gorman, 1990; Mol Cell Bio, 10:1805). Examples of such introns include, but are not limited to, the small t-intron, the SV40 intron, rabbit β-globin intron, chimeric intron (human β-globin donor and immunoglobulin heavy chain acceptor), chicken β-actin intron, hybrid intron (adenovirus/mouse immunoglobulin), and the minute virus of mice (MVM) VP intron. Any intron can be used in the expression cassette, so long as it comprises a splice donor/acceptor region recognized in mammalian cells, so that the intron can be spliced out of the resulting mRNA product. In one embodiment, the intron comprises, consists essentially of, or consists of an SV40 intron, e.g. a nucleotides 830-927 of SEQ ID NO: 2. In another embodiment, the intron comprises, consists essentially of, or consists of a chimeric intron, e.g. a nucleotides 937-1069 of SEQ ID NO: 1.

In some aspects, the promoter is a CMV promoter, a tMCK promoter, an MHCK7 promoter, or a CBA promoter. The CMV promoter consists of two elements, the CMV enhancer and CMV promoter itself, and is selected as a stable, constitutive, and ubiquitous promoter for transgene expression in all cell types. The MHCK7 promoter is highly specific for expression in skeletal muscles, including the diaphragm and heart tissues, and includes the following elements—αMHC enhancer, del63 MCK enhancer and MCK promoter—to minimize off-target effects in other tissues. The tMCK promoter drives expression in skeletal muscles and consists of a triple tandem of the MCK enhancer to the MCK basal promoter as described in Wang et al., (Gene Ther. 2008; 15 (22): 1489-1499). The CBA promoter drives high neural gene expression.

In some aspects, the CBA promoter comprises the nucleotides 495-749 of SEQ ID NO: 2. In some aspects, the tMCK promoter comprises nucleotides 165-884 of SEQ ID NO: 1. In some aspects, the CMV enhancer comprises nucleotides 209-463 of SEQ ID NO: 2.

### Gene Therapy Vectors

In some aspects, the disclosure includes a nanoparticle, extracellular vesicle, exosome, or vector comprising any of the nucleic acids of the disclosure or a combination of any one or more thereof for providing the BAG3 transgene. In some aspects, one or more copies of these sequences are combined into a single nanoparticle, extracellular vesicle, exosome, or vector.

The disclosure therefore includes vectors comprising a nucleic acid of the disclosure or a combination of nucleic acids of the disclosure. Embodiments of the disclosure utilize vectors (for example, viral vectors, such as adeno-associated virus (AAV), adenovirus, retrovirus, lentivirus, equine-associated virus, alphavirus, pox virus, herpes virus, herpes simplex virus, polio virus, sindbis virus, vaccinia virus or a synthetic virus, e.g., a chimeric virus, mosaic virus, or pseudotyped virus, and/or a virus that contains a foreign protein, synthetic polymer, nanoparticle to deliver the nucleic acids disclosed herein.

The disclosure provides a recombinant (r) AAV gene therapy vector comprising the nucleic acid comprising a polynucleotide encoding the BAG3 protein for use in treating neuroproteinopathies e.g., Amyotrophic lateral sclerosis (ALS) and myoproteinopathies e.g., LGMD1A (Myotilinopathy) in a subject in need thereof. AAV is unique in its safety profile, as the viral genome, once transduced into its carrier cell, remains stably expressed as an episomal DNA and only very rarely ever integrates into the host genome.

In some aspects, therefore, the disclosure utilizes AAV to deliver the BAG3 transgene, such as cDNA encoding the BAG3 protein. As used herein, the term “AAV” is a standard abbreviation for adeno-associated virus. AAV is a replication-deficient parvovirus, the single-stranded DNA genome of which is about 4.7 kb in length including structural elements known as the inverted terminal repeats (ITRs) that form dsDNA hairpin structures at each end. ITRs can form hairpin structures by self-annealing and they include the Rep binding element (RBE) and a terminal resolution site (TRS), which together constitute the AAV origin of replication. The ITRs are also required as packaging signals for genome encapsidation after replication. Construction of an rAAV vector genome requires only that the polynucleotides of interest (or transgenes) be flanked by the AAV terminal ITRs, and that the entire length of the vector genome does not exceed the upper packaging limit of approximately 5 kb. In any of the AAV genomes described herein, the genome comprises at least one an inverted terminal repeat (ITR), such as a mutant ITR or a wild type ITR.

An important limitation of recombinant adeno-associated virus (rAAV) vector efficiency is the requirement of host-cell-mediated synthesis of double-stranded DNA from the single-stranded genome. The ability to package double-stranded transgene DNA into AAV vectors termed self-complementary AAV (scAAV) vectors, offers the opportunity to bypass the requirement of viral second-strand DNA synthesis, overcoming said limitation. In scAAV vectors, deletion of the terminal resolution site from one of the ITRs prevents replication initiation by the Rep protein from the mutated end. The genome then replicates as an inverted dimer, which would self-anneal after viral uncoating (McCarty et al. 2001. Gene Ther 8:1248-1254, McCarty et al. 2003. Gene Ther 10:2112-2118, Wang et al. 2003 Gene Ther. 10 (26): 2105-2111). Compared with single-strand AAV (ssAAV) vectors, scAAV vectors have been shown to improve transduction efficiency in vivo however, they reduce the cloning capacity of AAVs by half i.e., to 2.2 kB.

Exemplary ITR sequences may be 130 base pairs in length or 141 base pairs in length, such as the ITR sequence comprising nucleotides 1-128 or SEQ ID NO: 1 or nucleotides 1-128 of SEQ ID NO: 2, which is an exemplary 5′ ITR, and nucleotides 3118-3245 of SEQ ID NO: 1 or nucleotides 2987-3114 of SEQ ID NO: 2, which is an exemplary 3′ ITR, which contains a deletion of the terminal resolution site. In any of the AAV genomes describe herein, the genome comprises at least one inverted terminal repeat (ITR), such as a mutant ITR or a wild type ITR.

Such AAV vectors can be replicated and packaged into infectious viral particles when present in a host cell that has been transfected with a vector encoding and expressing rep and cap gene products. AAV is a single-stranded replication-deficient DNA parvovirus that grows only in cells in which certain functions are provided by a co-infecting helper virus. The genome of AAV is about 4.7 kb in length including 145 nucleotide inverted terminal repeat (ITRs). There are multiple serotypes of AAV that have been characterized. General information and reviews of AAV can be found in, for example, Carter, 1989, Handbook of Parvoviruses, Vol. 1, pp. 169-228, and Berns, 1990, Virology, pp. 1743-1764, Raven Press, (New York). However, it is fully expected that these same principles will be applicable to additional AAV serotypes since it is well known that the various serotypes are quite closely related, both structurally and functionally, even at the genetic level. (See, for example, Blacklowe, 1988, pp. 165-174 of Parvoviruses and Human Disease, J. R. Pattison, ed.; and Rose, Comprehensive Virology 3:1-61 (1974). For example, all AAV serotypes apparently exhibit very similar replication properties mediated by homologous rep genes; and all bear three related capsid proteins such as those expressed in AAV2. The degree of relatedness is further suggested by heteroduplex analysis which reveals extensive cross-hybridization between serotypes along the length of the genome; and the presence of analogous self-annealing segments at the termini that correspond to “inverted terminal repeat sequences” (ITRs). The similar infectivity patterns also suggest that the replication functions in each serotype are under similar regulatory control.

There are multiple serotypes of AAV. The nucleotide sequences of the genomes of the AAV serotypes are known. For example, the nucleotide sequence of the AAV serotype 2 (AAV2) genome is presented in Srivastava et al., J Virol, 45:555-564 (1983) as corrected by Ruffing et al., J Gen Virol, 75:3385-3392 (1994). As other examples, the complete genome of AAV-1 is provided in GenBank Accession No. NC_002077; the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively (see also U.S. Pat. Nos. 7,282,199 and 7,790,449 relating to AAV-8); the AAV-9 genome is provided in Gao et al., J. Virol., 78:6381-6388 (2004); the AAV-10 genome is provided in Mol. Ther., 13 (1): 67-76 (2006); and the AAV-11 genome is provided in Virology, 330 (2): 375-383 (2004). Cloning of the AAVrh.74 serotype is described in Rodino-Klapac., et al. Journal of translational medicine 5, 45 (2007). Cis-acting sequences directing viral DNA replication (rep), encapsidation/packaging and host cell chromosome integration are contained within the ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (e.g., at AAV2 nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158:97-129 (1992).

Cis-acting sequences directing viral DNA replication (rep), encapsidation/packaging and host cell chromo-some integration are contained within the AAV ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (at nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158:97-129 (1992).

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is infectious as cloned DNA in plasmids which makes construction of recombinant genomes feasible. Furthermore, because the signals directing AAV replication, genome encapsidation and integration are contained within the ITRs of the AAV genome, some or all of the internal approximately 4.3 kb of the genome (encoding replication and structural capsid proteins, rep-cap) may be replaced with foreign DNA such as a gene expression cassette containing a promoter, a DNA of interest and a polyadenylation signal. The rep and cap proteins may be provided in trans. Another significant feature of AAV is that it is an extremely stable virus. It easily withstands the conditions used to inactivate adenovirus (56° C. to 65° C. for several hours), making cold preservation of AAV less critical. AAV may even be lyophilized. Finally, AAV-infected cells are not resistant to superinfection.

In some aspects, the AAV lacks rep and cap genes. In some aspects, the AAV is a recombinant linear AAV (rAAV), a single-stranded AAV (ssAAV), or a recombinant self-complementary AAV (scAAV). The self-complementary (sc) technology allows for binding of the single-stranded viral DNA genome onto itself, thereby priming second strand DNA synthesis. This sc element both quickens and strengthens gene expression relative to constructs lacking the sc element.

Advances in AAV vectors have led to safer and more efficient viral vehicles to deliver therapeutic transgenes in a single injection, and gene therapy is now a favorable therapeutic intervention for monogenic diseases. AAV vectors can provide long-term expression of gene products in post-mitotic target tissues. Thus, current AAV-based strategies may only require one-time vector administration.

Recombinant AAV genomes of the disclosure comprise one or more AAV ITRs flanking a polynucleotide encoding, for example, one or more MPZ inhibitory RNAs or MPZ miRNAs. The genomes of the rAAV provided herein either further comprise an RNAi-resistant replacement MPZ gene, or the RNAi-resistant replacement MPZ gene is present in a separate rAAV. The miRNA- and replacement MPZ-encoding polynucleotides are operatively linked to transcriptional control DNAs, for example promoter DNAs, which are functional in a target cell. Commercial providers such as Ambion Inc. (Austin, TX), Darmacon Inc. (Lafayette, CO), InvivoGen (San Diego, CA), and Molecular Research Laboratories, LLC (Herndon, VA) generate custom inhibitory RNA molecules. In addition, commercial kits are available to produce custom siRNA molecules, such as SILENCER™ siRNA Construction Kit (Ambion Inc., Austin, TX) or psiRNA System (InvivoGen, San Diego, CA).

In some aspects, the AAV is AAV1, AAV9 or AAVrh.74. AAV9 has become the most widely used vector for muscular and/or neurological indications with an established safety profile in the clinic. Intrathecal administration of AAV9 permits dissemination of transgenes throughout the nervous system and is currently approved by FDA for spinal muscular atrophy (SMA, NCT03381729), and in trials for the treatment of neuronal ceroid lipofuscinosis 3 (CLN3, NCT03770572), CLN6 (NCT02725580), giant axonal neuropathy (GAN, NCT02362438), mucopolysaccharidoses types 3A (NCT02716246) and 3B (NCT03315182), and exon 2 duplications in the DMD gene (NCT04240314). Such features make AAV9 an ideal gene delivery method for treatment of disorders where muscle and heart are the most affected organs. It has been shown that AAV9 can also target Schwann cells, and other peripheral neuropathies. More importantly, AAV9 was reported to transduce Schwann cells in large animals and non-human primates, indicating that it is a desirable viral vector for clinical applications requiring delivery of therapeutic genes into the human Schwann cells. Finally, data from studies in other models of muscle disease show that an AAV9 vector efficiently transfects skeletal muscle, heart, and diaphragm in mice and non-human primates.

In various aspects, the AAV is AAVrh.74. Cloning of the AAVrh.74 serotype is described in Rodino-Klapac., et al. Journal of translational medicine 5, 45 (2007). Isolated from rhesus macaque, AAVrh.74, has good transduction properties with high tropism for skeletal and cardiac muscle and it also has lower pre-existing human population immunity compared to AAV2. AAVrh.74 is being used for muscular indications and is being trialed for the treatment of Limb-Girdle Muscular dystrophy, type 2E (LGMD2E, NCT03652259) where it is being delivered intravenously, Dysferlinopathies e.g., limb girdle muscular dystrophy type 2B (LGMD2B) through intramuscular injection (NCT02710500), and Duchenne Muscular Dystrophy delivered systemically through a peripheral limb vein injection (NCT03375164).

In various aspects, the AAV is AAV1. AAV1 is commonly used for treatment of neuromuscular disorders. GLYBERA® the world's first approved viral gene therapy used AAV1 as a vector to deliver an intact copy of the human lipoprotein lipase gene by intramuscular administration in patients with familial lipoprotein lipase deficiency (LPLD). AAV1 is also in clinical trials for the treatment of Frontotemporal dementia (NCT04747431) direct administration into the CSF via intra-cisterna magna (ICM) injection, Charcot-Marie-Toothneuropathy type 1A (NCT03520751) via intramuscular injections, and Congestive heart failure (NCT04703842) via intracoronary delivery to the heart.

Taken together, this highlights the relative safety of gene therapy in diseases that require localized or systemic delivery.

DNA plasmids of the disclosure comprise rAAV genomes of the disclosure. In some aspects, the DNA plasmids are transferred to cells permissible for infection with a helper virus of AAV (e.g., adenovirus, E1-deleted adenovirus or herpes virus) for assembly of the rAAV genome into infectious viral particles. Thus, in some aspects, the disclosure includes AAV vectors to deliver therapeutic agents into a cell. In some aspects, the cell is a neuronal cell. In some aspects, the neuronal cell is a Schwann cell.

An “AAV virion” or “AAV viral particle” or “AAV particle” or “AAV vector particle” refers to a viral particle composed of at least one AAV capsid protein and an encapsidated polynucleotide AAV vector. If the particle comprises a heterologous polynucleotide (i.e. a polynucleotide other than a wild-type AAV genome such as a transgene to be delivered to a mammalian cell), it is typically referred to as an “AAV vector particle” or simply an “AAV vector”. Thus, production of AAV vector particle necessarily includes production of AAV vector, as such a vector is contained within an AAV vector particle. Techniques to produce rAAV particles, in which an AAV genome to be packaged, rep and cap genes, and helper virus functions are provided to a cell are standard in the art. Production of rAAV requires that the following components are present within a single cell (denoted herein as a packaging cell): a rAAV genome, AAV rep and cap genes separate from (i.e., not in) the rAAV genome, and helper virus functions. The AAV rep genes may be from any AAV serotype for which recombinant virus can be derived and may be from a different AAV serotype than the rAAV genome ITRs, including, but not limited to, AAV serotypes AAV1, AAV2, AAV3, AAV4, AAV5, AAV6, AAV7, AAV8, AAV9, AAV10, AAV11, AAV12, AAV13, AAVrh.74, AAVrh.8, or AAVrh. 10, AAVAnc80, AAV7m8, AAV2/1, AAV2/8, AAV2/9, or AAVMYO and their derivatives. In some aspects, AAV DNA in the rAAV genomes is from any AAV serotype for which a recombinant virus can be derived including, but not limited to, AAV serotypes AAV is AAV1, AAV2, AAV3, AAV4, AAV5, AAV6, AAV7, AAV8, AAV9, AAV10, AAV11, AAV12, AAV13, AAVrh.74, AAVrh.8, or AAVrh. 10, AAVAnc80, AAV7m8, AAV2/1, AAV2/8, AAV2/9, or AAVMYO, and their derivatives. Other types of rAAV variants, including those for example with capsid mutations, are also included in the disclosure. Such variants include, but are not limited to, MyoAAV or AAVMYO, and other variants as described, for example, in Marsic et al., Molecular Therapy 22 (11): 1900-1909 (2014); Weismann, J., et al., Nat Commun 11 (1): 5432 (2020) and Tabebordbar, M. et al., Cell 184 (19): 4919-4938 e22 (2021), which are incorporated for use herein by reference in their entirety. As noted above, the nucleotide sequences of the genomes of various AAV serotypes are known in the art. Use of cognate components is specifically contemplated. Production of pseudotyped rAAV is disclosed in, for example, WO 01/83692 which is incorporated by reference herein in its entirety.

In some embodiments, the viral vector is a pseudotyped AAV, containing ITRs from one AAV serotype and capsid proteins from a different AAV serotype. In some embodiments, the pseudo-typed AAV is AAV2/9 (i.e., an AAV containing AAV2 ITRs and AAV9 capsid proteins). In some embodiments, the pseudotyped AAV is AAV2/8 (i.e., an AAV containing AAV2 ITRs and AAV8 capsid proteins). In some embodiments, the pseudotyped AAV is AAV2/1 (i.e., an AAV containing AAV2 ITRs and AAV1 capsid proteins).

In some embodiments, the AAV contains a recombinant capsid protein, such as a capsid protein containing a chimera of one or more of capsid proteins from AAV1, AAV2, AAV3, AAV4, AAV5, AAV6, AAV7, AAV8, AAV9, AAV10, AAV11, AAV12, AAV13, AAVrh.74, AAVrh.8, or AAVrh. 10, AAVAnc80, AAV7m8, AAV2/1, AAV2/8, AAV2/9, or AAVMYO and their derivatives. Other types of rAAV variants, for example rAAV with capsid mutations, are also contemplated. See, for example, Marsic et al., Molecular Therapy, 22 (11): 1900-1909 (2014). The nucleotide sequences of the genomes of various AAV serotypes are known in the art.

Multiple studies have demonstrated long-term (>1.5 years) recombinant AAV-mediated protein expression in muscle. See, Clark et al., Hum Gene Ther, 8:659-669 (1997); Kessler et al., Proc Nat. Acad Sc. USA, 93:14082-14087 (1996); and Xiao et al., J Virol, 70:8098-8108 (1996). See also, Chao et al., Mol Ther, 2:619-623 (2000) and Chao et al., Mol Ther, 4:217-222 (2001). Moreover, because muscle is highly vascularized, recombinant AAV transduction has resulted in the appearance of transgene products in the systemic circulation following intramuscular injection as described in Herzog et al., Proc Natl Acad Sci USA, 94:5804-5809 (1997) and Murphy et al., Proc Natl Acad Sci USA, 94:13921-13926 (1997). Moreover, Lewis et al., J Virol, 76:8769-8775 (2002) demonstrated that skeletal myofibers possess the necessary cellular factors for correct antibody glycosylation, folding, and secretion, indicating that muscle is capable of stable expression of secreted protein therapeutics.

Recombinant AAV genomes, in various aspects, comprise nucleic acids of the disclosure and one or more AAV ITRs flanking a nucleic acid molecule. AAV DNA in the rAAV genomes may be from any AAV serotype for which a recombinant virus can be derived including, but not limited to, AAV serotypes (e.g., AAV1, AAV2, AAV3, AAV4, AAV5, AAV6, AAV7, AAV8, AAV9, AAVRH10, AAVRH74, AAV11, AAV12, AAV13, or Anc80, AAV7m8 and their derivatives). Production of pseudotyped rAAV is disclosed in, for example, WO 01/83692. Other types of rAAV variants, for example rAAV with capsid mutations, are also contemplated. See, for example, Marsic et al., Molecular Therapy, 22 (11): 1900-1909 (2014). As noted in the Background section above, the nucleotide sequences of the genomes of various AAV serotypes are known in the art.

The provided recombinant AAV (i.e., infectious encapsidated rAAV particles) comprise a rAAV genome. The term “rAAV genome” refers to a polynucleotide sequence that is derived from a native AAV genome that has been modified. In some embodiments, the rAAV genome has been modified to remove the native cap and rep genes. In some embodiments, the rAAV genome comprises the endogenous 5′ and 3′ inverted terminal repeats (ITRs). In some embodiments, the rAAV genome comprises ITRs from an AAV serotype that is different from the AAV serotype from which the AAV genome was derived. In some embodiments, the rAAV genome comprises a transgene of interest flanked on the 5′ and 3′ ends by inverted terminal repeat (ITR). In some embodiments, the rAAV genome comprises a “gene cassette.” In exemplary embodiments, the genomes of both rAAV lack AAV rep and cap DNA, that is, there is no AAV rep or cap DNA between the ITRs of the genomes.

DNA plasmids of the disclosure comprise rAAV genomes of the disclosure. The DNA plasmids are transferred to cells permissible for infection with a helper virus of AAV (e.g., adenovirus, E1-deleted adenovirus or herpesvirus) for assembly of the rAAV genome into infectious viral particles. Techniques to produce rAAV particles, in which an AAV genome to be packaged, rep and cap genes, and helper virus functions are provided to a cell are standard in the art. Production of rAAV requires that the following components are present within a single cell (denoted herein as a packaging cell): a rAAV genome, AAV rep and cap genes separate from (i.e., not in) the rAAV genome, and helper virus functions. The AAV rep and cap genes may be from any AAV serotype for which recombinant virus can be derived and may be from a different AAV serotype than the rAAV genome ITRs, including, but not limited to, AAV serotypes AAV9, AAV1, AAV2, AAV3, AAV4, AAV5, AAV6, AAV7, AAVrh.74, AAV8, AAV10, AAV11, AAV12 and AAV13. Production of pseudotyped rAAV is disclosed in, for example, WO 01/83692 which is incorporated by reference herein in its entirety.

A method of generating a packaging cell is to create a cell line that stably expresses all the necessary components for AAV particle production. For example, a plasmid (or multiple plasmids) comprising a rAAV genome lacking AAV rep and cap genes, AAV rep and cap genes separate from the rAAV genome, and a selectable marker, such as a neomycin resistance gene, are integrated into the genome of a cell. AAV genomes have been introduced into bacterial plasmids by procedures such as GC tailing (Samulski et al. 1982. Proc Natl Acad Sci USA. 79, 2077-2081), addition of synthetic linkers containing restriction endonuclease cleavage sites (Laughlin et al. 1983. Gene. 23, 65-73) or by direct, blunt-end ligation (Senapathy et al. 1984. J Biol Chem. 259, 4661-4666). The packaging cell line is then infected with a helper virus such as adenovirus. The advantages of this method are that the cells are selectable and are suitable for large-scale production of rAAV. Other examples of suitable methods employ adenovirus or baculovirus rather than plasmids to introduce rAAV genomes and/or rep and cap genes into packaging cells.

General principles of rAAV production are reviewed in, for example, Carter, 1992, Current Opinions in Biotechnology, 1533-539; and Muzyczka, 1992, Curr. Topics in Microbial, and Immunol., 158:97-129. Various approaches are described in Ratschin et al., Mol. Cell. Biol. 4:2072 (1984); Hermonat et al., Proc. Natl. Acad. Sci. USA, 81:6466 (1984); Tratschin et al., Mol. Cell. Biol. 5:3251 (1985); Mclaughlin et al., J. Virol., 62:1963 (1988); and Lebkowski et al., Mol. Cell. Biol., Oct; 8 (10): 3988-96 (1988); Samulski et al., J. Virol., 63:3822-3828 (1989); U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al. Vaccine 13:1244-1250 (1995); Paul et al. Human Gene Therapy 4:609-615 (1993); Clark et al. Gene Therapy 3:1124-1132 (1996) 51; U.S. Pat. Nos. 5,786,211; 5,871,982; and 6,258,595. The foregoing documents are hereby incorporated by reference in their entirety herein, with particular emphasis on those sections of the documents relating to rAAV production. The production and use of self-complementary (sc) rAAV are specifically contemplated and exemplified.

The disclosure thus provides packaging cells that produce infectious rAAV. In one embodiment, packaging cells are stably transformed cancer cells, such as Hela cells, 293 cells and PerC.6 cells (a cognate 293 line). In another embodiment, packaging cells are cells that are not transformed cancer cells, such as low passage 293 cells (human fetal kidney cells transformed with E1 of adenovirus), MRC-5 cells (human fetal fibroblasts), WI-38 cells (human fetal fibroblasts), Vero cells (monkey kidney cells) and FRhL-2 cells (rhesus fetal lung cells).

In some aspects, rAAV is purified by methods standard in the art, such as by column chromatography or cesium chloride gradients. Methods for purifying rAAV vectors from helper virus are known in the art and include methods disclosed in, for example, Clark et al., Hum. Gene Ther., 10 (6): 1031-1039 (1999); Schenpp and Clark, Methods Mol. Med., 69 427-443 (2002); U.S. Pat. No. 6,566,118 and WO 98/09657.

### Compositions and Methods of Use

Compositions comprising the nucleic acids and viral vectors of the disclosure are provided. Compositions comprising delivery vehicles (such as rAAV) described herein are provided. In various aspects, such compositions also comprise a pharmaceutically acceptable carrier. In some aspects, a pharmaceutically acceptable carrier is a diluent, excipient, or buffer. The compositions may also comprise other ingredients, such as adjuvants.

Acceptable carriers, diluents, excipients, and adjuvants are nontoxic to recipients and are preferably inert at the dosages and concentrations employed, and include buffers such as phosphate, citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, pluronics or polyethylene glycol (PEG).

Sterile injectable solutions are prepared by incorporating rAAV in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique that yield a powder of the active ingredient plus any additional desired ingredient from the previously sterile-filtered solution thereof.

Dosages of rAAV to be administered in methods of the disclosure will vary depending, for example, on the particular rAAV, the mode of administration, the time of administration, the treatment goal, the individual, and the cell type(s) being targeted, and may be determined by methods standard in the art. Dosages may be expressed in units of viral genomes (vg). Dosages contemplated herein include about 1×10⁷, 1×10⁸, 1×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 1×10¹¹, about 1×10¹², about 1×10¹³, about 1.1×10¹³, about 1.2×10¹³, about 1.3×10¹³, about 1.5×10¹³, about 2×10¹³, about 2.5×10¹³, about 3×10¹³, about 3.5×10¹³, about 4×10¹³, about 4.5×10¹³, about 5×10¹³, about 6×10¹³, about 7×10¹³, about 8×10¹³, about 9×10¹³, about 1×10¹⁴, about 2×10¹⁴, about 3×10¹⁴, about 4×10¹⁴, about 5×10¹⁴, about 1×10¹⁵, to about 1×10¹⁶, or more total viral genomes.

Dosages of about 1×10⁹ to about 1×10¹⁰, about 5×10⁹ to about 5×10¹⁰, about 1×10¹⁰ to about 1×10¹¹, about 1×10¹¹ to about 1×10¹⁵ vg, about 1×10¹² to about 1×10¹⁵ vg, about 1×10¹² to about 1×10¹⁴ vg, about 1×10¹³ to about 6×10¹⁴ vg, about 1×10¹³ to about 1×10¹⁵ vg and about 6×10¹³ to about 1.0×10¹⁴ vg are also contemplated. One dose exemplified herein is 1×10¹³ vg administered via intravenous or intraperitoneal delivery.

Dosages are also may be expressed in units of vg/kg. Dosages contemplated herein include about 1×10⁷ vg/kg, 1×10⁸ vg/kg, 1×10⁹ vg/kg, 5×10⁹ vg/kg, 6×10⁹ vg/kg, 7×10⁹ vg/kg, 8×10⁹ vg/kg, 9×10⁹ vg/kg, 1×10¹⁰ vg/kg, 2×10¹⁰ vg/kg, 3×10¹⁰ vg/kg, 4×10¹⁰ vg/kg, 5×10¹⁰ vg/kg, 1×10¹¹ vg/kg, about 1×10¹² vg/kg, about 1×10¹³ vg/kg, about 1.1×10¹³ vg/kg, about 1.2×10¹³ vg/kg, about 1.3×10¹³ vg/kg, about 1.5×10¹³ vg/kg, about 2×10¹³ vg/kg, about 2.5×10¹³ vg/kg, about 3×10¹³ vg/kg, about 3.5×10¹³ vg/kg, about 4×10¹³ vg/kg, about 4.5×10¹³ vg/kg, about 5×10¹³ vg/kg, about 6×10¹³ vg/kg, about 7×10¹³, about 8×10¹³, about 9×10¹³, about 1×10¹⁴ vg/kg, about 2×10¹⁴ vg/kg, about 3×10¹⁴ vg/kg, about 4×10¹⁴ vg/kg about 5×10¹⁴ vg/kg, about 1×10¹⁵ vg/kg, to about 1×10¹⁶ vg/kg.

Dosages of about 1×10⁹ vg/kg to about 1×10¹⁰ vg/kg, about 5×10⁹ vg/kg to about 5×10¹⁰ vg/kg, about 1×10¹⁰ vg/kg to about 1×10¹¹ vg/kg, about 1×10¹¹ vg/kg to about 1×10¹⁵ vg/kg, about 1×10¹² vg/kg to about 1×10¹⁵ vg/kg, about 1×10¹² vg/kg to about 1×10¹⁴ vg/kg, about 1×10¹³ vg/kg to about 2×10¹⁴ vg/kg, about 1×10¹³ vg/kg to about 1×10¹⁵ vg/kg and about 6×10¹³ vg/kg to about 1.0×10¹⁴ vg/kg are also contemplated. One dose exemplified herein is 1×10¹³ vg/g administered via intravenous or intraperitoneal delivery.

Transduction or transfection of cells with rAAV of the disclosure results in sustained expression of the BAG3 gene/protein. As used herein, the terms “transduction” and “transfection” are used interchangeably. The term “transduction” or “transfection” is used to refer to, as an example, the administration/delivery of the BAG3 gene to a target cell either in vivo or in vitro, via a replication-deficient rAAV described herein resulting in the expression of the BAG3 gene/protein by the target cell. The disclosure thus provides methods of administering/delivering rAAV which express the BAG3 gene to a cell or to a subject. In some aspects, the subject is a mammal. In some aspects, the mammal is a human. These methods include transducing cells and tissues (including, but not limited to, peripheral motor neurons, sensory motor neurons, neurons, Schwann cells, and other tissues or organs, such as muscle, heart, liver and brain) with one or more rAAV described herein. Transduction may be carried out with gene cassettes comprising cell-specific control elements.

Methods of transducing a target cell with a delivery vehicle (such as a nanoparticle, extracellular vesicle, exosome, or vector (e.g., rAAV)), in vivo or in vitro, are provided. The in vivo methods comprise the step of administering an effective dose, or effective multiple doses, of a composition comprising a delivery vehicle (such as rAAV) to an animal (including a human subject or patient) in need thereof. If the dose is administered prior to development of a disorder/disease, the administration is prophylactic. If the dose is administered after the development of a disorder/disease, the administration is therapeutic. An effective dose is a dose that alleviates (eliminates or reduces) at least one symptom associated with the disorder/disease state being treated, that slows or prevents progression to a disorder/disease state, that slows or prevents progression of a disorder/disease state, that diminishes the extent of disease, that results in remission (partial or total) of disease, and/or that prolongs survival. Thus, methods are provided of administering an effective dose (or doses, administered essentially simultaneously or doses given at intervals) of rAAV described herein to a subject in need thereof.

Provided herein are medicaments and methods for treating, ameliorating, or preventing a disease or disorder associated with misfolded protein or protein aggregates or a disease or disorder associated with a mutant BAG3 gene or aberrant BAG3 gene expression. Molecular, biochemical, histological, and functional outcome measures demonstrate the therapeutic efficacy of the methods. The level of human BAG3 transcript in animals in can be confirmed by RT-PCR and/or RNAseq. The BAG3 protein expression level in skeletal muscles, including heart and diaphragm can be assessed using western blotting. BAG3 localization and proper co-localization with its binding partners in Z-discs as well as hallmarks of the pathology of muscles and inflammation can be confirmed by immunohistochemistry. To assess efficacy of potential treatment in mice, measurements of muscle contractile function can be performed using Aurora Whole Animal Muscle Test System. In patients, a variety of functional outcome measures may be used to assess successful treatment, including: 100 meter timed test, 10 meter walk/run test, North Star Ambulatory Assessment for limb girdle type muscular dystrophies (NSAD), Performance of Upper Limb (PUL) 2.0, and myometry assessments of force (including measures of shoulder abduction, elbow flexion/extension, and knee flexion/extensionforce).

In the methods of the disclosure, expression of the BAG3 protein is increased by at least 10, at least 20, at least 30, at least 40, at least 50, at least 60, at least 70, at least 80, at least 90, at least 95, at least 98 percent, at least 99 percent, or 100 percent.

### Combination Therapies

Combination therapies are also contemplated by the disclosure. Combination as used herein includes both simultaneous treatment and sequential treatments. Combinations of methods of the disclosure with standard medical treatments are specifically contemplated, as are combinations with novel therapies.

### Immunosuppressing Agent Combination Therapy

In some embodiments, the combination therapy comprises administering an immunosuppressing agent in combination with the gene therapy disclosed herein.

The immunosuppressing agent may be administered before or after the onset of an immune response to the rAAV in the subject after administration of the gene therapy. In addition, the immunosuppressing agent may be administered simultaneously with the gene therapy or the protein replacement therapy. The immune response in a subject includes an adverse immune response or an inflammatory response following or caused by the administration of rAAV to the subject. The immune response may be the production of antibodies in the subject in response to the administered rAAV.

Exemplary immunosuppressing agents include glucocorticosteroids, janus kinase inhibitors, calcineurin inhibitors, mTOR inhibitors, cyctostatic agents such as purine analogs, methotrexate and cyclophosphamide, inosine monophosphate dehydrogenase (IMDH) inhibitors, biologics such as monoclonal antibodies or fusion proteins and polypeptides, and di peptide boronic acid molecules, such as Bortezomib.

The immunosuppressing agent may be an anti-inflammatory steroid, which is a steroid that decreases inflammation and suppresses or modulates the immune system of the subject. Exemplary anti-inflammatory steroid are glucocorticoids such as prednisolone, betamethasone, dexamethasone, methotrexate, hydrocortisone, methylprednisolone, deflazacort, budesonide or prednisone.

Janus kinase inhibitors are inhibitors of the JAK/STAT signaling pathway by targeting one or more of the Janus kinase family of enzymes. Exemplary janus kinase inhibitors include tofacitinib, baricitinib, upadacitinib, peficitinib, and oclacitinib.

Calcineurin inhibitors bind to cyclophilin and inhibits the activity of calcineurin Exemplary calcineurine inhibitors includes cyclosporine, tacrolimus and picecrolimus.

mTOR inhibitors reduce or inhibit the serine/threonine-specific protein kinase mTOR. Exemplary mTOR inhibitors include rapamycin (also known as sirolimus), everolimus, and temsirolimus.

The immunosuppressing agents include immune suppressing macrolides. The term “immune suppressing macrolides” refer to macrolide agents that suppresses or modulates the immune system of the subject. A macrolide is a class of agents that comprise a large macrocyclic lactone ring to which one or more deoxy sugars, such as cladinose or desoamine, are attached. The lactone rings are usually 14-, 15-, or 16-membered. Macrolides belong to the polyketide class of agents and may be natural products. Examples of immunosuppressing macrolides include tacrolimus, pimecrolimus, and rapamycin (also known as sirolimus).

Purine analogs block nucleotide synthesis and include IMDH inhibitors. Exemplary purine analogs include azathioprine, mycophenolate such as mycophenolate acid or mycophenolate mofetil and lefunomide.

Exemplary immunosuppressing biologics include abatacept, adalimumab, anakinra, certolizumab, etanercept, golimumab, infliximab, ixekizumab, natalizumab, rituximab, secukinumab, tocilizumab, ustekinenumab, vedolizumab, basiliximab, belatacep, and daclizumab.

In particular, the immunosuppressing agent is an anti-CD20 antibody. The term anti-CD20 specific antibody refers to an antibody that specifically binds to or inhibits or reduces the expression or activity of CD20. Exemplary anti-CD20 antibodies include rituximab, ocrelizumab or ofatumumab.

Additional examples of immuosuppressing antibodies include anti-CD25 antibodies (or anti-IL2 antibodies or anti-TAC antibodies) such as basiliximab and daclizumab, and anti-CD3 antibodies such as muromonab-CD3, otelixizumab, teplizumab and visilizumab, anti-CD52 antibodies such as alemtuzumab.

One exemplary combination therapy is the delivery of rapamycin and rituximab prior to, or contemporaneous with, delivery of the AAV vector. Another exemplary combination therapy is the delivery of rapamycin, rituximab, and a corticosteroid, such as prednisone.

### Neurotrophic Factor (NTF) Combination Therapy

In some embodiments, the combination therapy comprises administering a neurotrophic factor (NTF) in combination with the gene therapy disclosed herein. NTFs are necessary to regulate several physiological processes such as neuronal differentiation and survival, axonal outgrowth and synapses maintenance, proliferation and differentiation of stem cells in the nervous system (Chao M V. 2003. Nat Rev Neurosci. 4 (4): 299-309). Moreover, reduced levels of NTFs have been reported in several neurodegenerative disorders (Connor B, Dragunow M. 1998. Brain Res Brain Res Rev. 27 (1): 1-39), including ALS (Anand et al. 1995. Nat Med. 1 (2): 168-72. 9-11, Lee et al. 1996 J Neuropathol Exp Neurol. 55 (8): 915-23, Ono et al. 1999. Eur Neurol. 42 (3): 163-8. 8092), suggesting that loss of trophic support could be important in disease pathophysiology. In the context of amyotrophic lateral sclerosis (ALS), it has long been hypothesized that lack of neurotrophic growth factors is one of the neurotoxic contributors to the disease that results in death of motor neurons. Moreover, previous studies in ALS mouse models e.g., SOD1^{G93A} mice demonstrated preclinical efficacy using BDNF, CNTF, and GDNF (reviewed in Gouel et al. 2019. Front Neurol. 10:835). Therefore, combination of NFTs and the BAG3 replacement therapy of the present disclosure represents a promising synergistic therapeutic strategy to treat neurodegenerative diseases resulting from protein aggregation such as ALS.

In some embodiments, the NTF is chosen from the group consisting of brain-derived growth factor (BDNF), neurotrophin-3, (NT-3), nerve growth factor (NGF), fibroblast growth factor (FGF) and glial cell-derived growth factor (GDNF). In some embodiments, the NFT is NT-3. NT-3 belongs to a family of growth factors called neurotrophins has been shown to be a key mediator of neuronal development during the early neurogenic period. as well as throughout adulthood. In humans, NT-3 is encoded by the NTF3 gene which is localized to chromosome 12p13 and encodes a 119 amino acid polypeptide that forms a homodimer (Maisonpierre et al. 1991. Genomics. 10:558-568). NT3 exerts its effect by binding to high-affinity tropomyosin receptor kinase (e.g., tropomyosin-related kinase C receptor (TrkC)) receptors and a low-affinity p75 neurotrophin receptor (p75NTR). The binding of NTF3 to TrkC induces PI3K/Akt and RAS/ERK signaling pathways that regulate cell growth, survival, and differentiation (Chao M V. 2003. Nat Rev Neurosci 4 (4): 299-309, Liot et al. Exp Neurol. 187 (1): 38-46). Functional deficiency in NTF3 has been shown to cause severe neuronal deficits and early postnatal death in mice (Conover J C, Yancopoulos G D. 1997. Rev Neurosci. 8 (1): 13-27). Furthermore, motor neurons derived from conditional Ntf3-knockout embryos showed increased apoptosis and abnormal projection of the central-branch-innervating motor neuron, suggesting that NTF3 is critically important for survival and axonal projection of neurons (Usin et al. 2012. Development 139 (6): 1125-1132).

In some embodiments, the NT-3 polynucleotide in a rAAV genome is the NT-3 cDNA set out in SEQ ID NO: 9. In some embodiments, the NT-3 polynucleotide in a rAAV genome is the NT-3 cDNA set out in Genbank Accession #NM_001102654 or the NT-3 cDNA sequence set out as SEQ ID NO: 9, or is a polynucleotide having 80%, 81%, 82%, 83%, 84%, 85%, 86%, 87%, 88%, 89%, 90%, 91%, 92%, 93%, 94%, 95%, 96%, 97%, 98% or 99% sequence identity to the NT-3 cDNA. In some embodiments, the NT-3 polynucleotide encodes the same NT-3 polypeptide as the polypeptide encoded by NT-3 cDNA of SEQ ID NO: 9. The amino acid sequence of the NT-3 polypeptide encoded by the NT-3 cDNA set out as SEQ ID NO: 9. An amino acid sequence alteration can be, for example, a substitution, a deletion, or an insertion of one or more amino acids, preferably conservative substitutions. A NT-3 polypeptide can have any combination of amino acid substitutions, deletions or insertions where activity of the polypeptide is retained. In one aspect, a NT-3 polypeptide can have a number of amino acid alterations such that its amino acid sequence shares at least 60, 70, 80, 85, 90, 95, 97, 98, 99 or 99.5% identity with the amino acid sequence (SEQ ID NO: 10) encoded by NT-3 cDNA set out as SEQ ID NO:9 or provided as Genbank Accession #NM_001102654.

In some embodiments, the rAAV genome is the sc.AAV1.tMCK.NTF3 genome. An exemplary sc.AAV1.tMCK.NT-3 is disclosed in international publication number: WO2019079755, incorporated by reference herein in its entirety.

In yet another aspect, an isolated nucleic acid comprising the nucleotide sequence depicted in SEQ ID NO: 12 is provided. Also provided is an isolated nucleic acid comprising, in order from 5′ to 3′: (i) a first AAV2 inverted terminal repeat sequence (ITR) (; (ii) a muscle creatine kinase promoter sequence; (iii) a nucleotide sequence encoding a human NT-3 polypeptide (SEQ ID NO: 9); and (iv) a second AAV2 ITR sequence, wherein the human NT-3 polypeptide has an amino acid sequence that is at least 90% identical to SEQ ID NO: 10, is 100% identical to SEQ ID NO:10.

Recombinant AAV comprising the foregoing nucleic acids are contemplated as well as rAAV comprising a nucleotide sequence that is at least at least 60, 70, 80, 85, 90, 95, 97, 98, 99 or 99.5% identity identical to the nucleotide sequence depicted in SEQ ID NO: 12.

Administration of an effective dose of a nucleic acid, nanoparticle, extracellular vesicle, exosome, viral vector, or composition of the disclosure may be by routes standard in the art including, but not limited to, intramuscular, parenteral, intravascular, intravenous, oral, buccal, nasal, pulmonary, intracranial, intracerebroventricular, intrathecal, intraosseous, intraocular, rectal, or vaginal. In various aspects, an effective dose is delivered by a combination of routes. For example, in various aspects, an effective dose is delivered intravenously and/or intramuscularly, or intravenously and intracerebroventricularly, and the like. In some aspects, an effective dose is delivered in sequence or sequentially. In some aspects, an effective dose is delivered simultaneously. Route(s) of administration and serotype(s) of AAV components of the rAAV (in particular, the AAV ITRs and capsid protein) of the disclosure may be chosen and/or matched by those skilled in the art taking into account the infection and/or disease state being treated and the target cells/tissue(s) that are to express the miRNAs.

In particular, actual administration of delivery vehicle (such as rAAV) may be accomplished by using any physical method that will transport the delivery vehicle (such as rAAV) into a target cell of a subject. Administration includes, but is not limited to, injection into muscle, the bloodstream and/or directly into the nervous system or liver. Simply resuspending a rAAV in phosphate buffered saline has been demonstrated to be sufficient to provide a vehicle useful for muscle tissue expression, and there are no known restrictions on the carriers or other components that can be co-administered with the rAAV (although compositions that degrade DNA should be avoided in the normal manner with rAAV). Capsid proteins of a rAAV may be modified so that the rAAV is targeted to a particular target tissue of interest such as neurons. See, for example, WO 02/053703, the disclosure of which is incorporated by reference herein. Pharmaceutical compositions can be prepared as injectable formulations or as topical formulations to be delivered to the muscles by transdermal transport. Numerous formulations for both intramuscular injection and transdermal transport have been previously developed and can be used in the practice of the methods of the disclosure. The delivery vehicle (such as rAAV) can be used with any pharmaceutically acceptable carrier for ease of administration and handling.

A dispersion of delivery vehicle (such as rAAV) can also be prepared in glycerol, sorbitol, liquid polyethylene glycols and mixtures thereof and in oils. Under ordinary conditions of storage and use, these preparations contain a preservative to prevent the growth of microorganisms. In this connection, the sterile aqueous media employed are all readily obtainable by standard techniques well-known to those skilled in the art.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringeability exists. It must be stable under the conditions of manufacture and storage and must be preserved against the contaminating actions of microorganisms such as bacteria and fungi. The carrier can be a solvent or dispersion medium containing, for example, water, ethanol, polyol (for example, glycerol, propylene glycol, liquid polyethylene glycol, sorbitol and the like), suitable mixtures thereof, and vegetable oils. The proper fluidity can be maintained, for example, by the use of a coating such as lecithin, by the maintenance of the required particle size in the case of a dispersion and by the use of surfactants. The prevention of the action of microorganisms can be brought about by various antibacterial and antifungal agents, for example, parabens, chlorobutanol, phenol, sorbic acid, thimerosal and the like. In many cases it will be preferable to include isotonic agents, for example, suMPZ or sodium chloride. Prolonged absorption of the injectable compositions can be brought about by use of agents delaying absorption, for example, aluminum monostearate and gelatin.

Sterile injectable solutions are prepared by incorporating rAAV in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique that yield a powder of the active ingredient plus any additional desired ingredient from the previously sterile-filtered solution thereof.

The disclosure also provides kits for use in the treatment of a disease or disorder described herein. Such kits include at least a first sterile composition comprising any of the nucleic acids described herein above or any of the viral vectors described herein above in a pharmaceutically acceptable carrier. Another component is optionally a second therapeutic agent for the treatment of the disorder along with suitable container and vehicles for administrations of the therapeutic compositions. The kits optionally comprise solutions or buffers for suspending, diluting or effecting the delivery of the first and second compositions.

In one embodiment, such a kit includes the nucleic acids or vectors in a diluent packaged in a container such as a sealed bottle or vessel, with a label affixed to the container or included in the package that describes use of the nucleic acids or vectors. In one embodiment, the diluent is in a container such that the amount of headspace in the container (e.g., the amount of air between the liquid formulation and the top of the container) is very small. Preferably, the amount of headspace is negligible (i.e., almost none).

In some aspects, the formulation comprises a stabilizer. The term “stabilizer” refers to a substance or excipient which protects the formulation from adverse conditions, such as those which occur during heating or freezing, and/or prolongs the stability or shelf-life of the formulation in a stable state. Examples of stabilizers include, but are not limited to, stabilizers, such as sucrose, lactose and mannose; sugar alcohols, such as mannitol; amino acids, such as glycine or glutamic acid; and proteins, such as human serum albumin or gelatin.

In some aspects, the formulation comprises an antimicrobial preservative. The term “antimicrobial preservative” refers to any substance which is added to the composition that inhibits the growth of microorganisms that may be introduced upon repeated puncture of the vial or container being used. Examples of antimicrobial preservatives include, but are not limited to, substances such as thimerosal, 2-phenoxyethanol, benzethonium chloride, and phenol.

In some aspects, the kit comprises a label and/or instructions that describes use of the reagents provided in the kit. The kits also optionally comprise catheters, syringes or other delivering devices for the delivery of one or more of the compositions used in the methods described herein.

This entire document is intended to be related as a unified disclosure, and it should be understood that all combinations of features described herein are contemplated, even if the combination of features are not found together in the same sentence, or paragraph, or section of this document. The disclosure also includes, for instance, all embodiments of the disclosure narrower in scope in any way than the variations specifically mentioned above. With respect to aspects of the disclosure described as a genus, all individual species are considered separate aspects of the disclosure. With respect to aspects of the disclosure described or claimed with “a” or “an,” it should be understood that these terms mean “one or more” unless context unambiguously requires a more restricted meaning. If aspects of the disclosure are described as “comprising” a feature, embodiments also are contemplated “consisting of” or “consisting essentially of” the feature.

All publications, patents and patent applications cited in this specification are herein incorporated by reference as if each individual publication or patent application were specifically and individually indicated to be incorporated by reference in its entirety to the extent that it is not inconsistent with the disclosure.

It is understood that the examples and embodiments described herein are for illustrative purposes only and that various modifications or changes in light thereof will be suggested to persons skilled in the art and are to be included within the spirit and purview of this application and scope of the appended claims. Thus, the following examples are provided by way of illustration and not limitation.

Described numerical ranges are inclusive of each integer value within each range and inclusive of the lowest and highest stated integer.

### EXAMPLES

### Example 1

### Construction of BAG3 Expressing Constructs

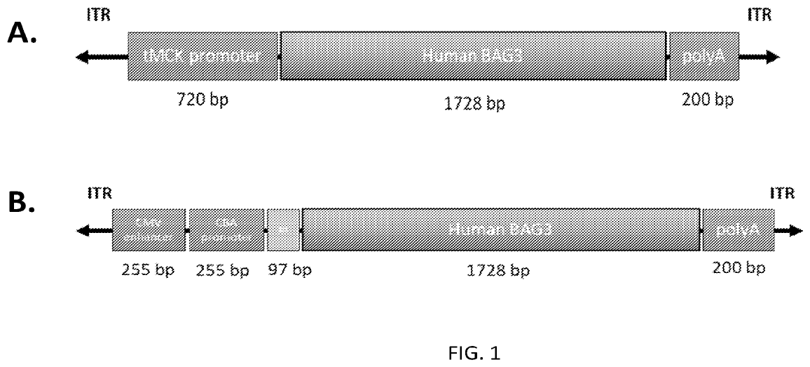

AAV genome constructs encoding BAG3 used in the following examples are non-replicating recombinant adeno-associated virus termed ssAAVrh74.tMCK.BAG3 (FIG. 1A) and ssAAV9.CMV.CBA.BAG3 (FIG. 1B).

First, ss.pAAV.CMV.BAG3 plasmid was cloned and the AAV serotype 9 vector carrying human BAG3 cDNA under CMV promoter was produced using standard methodology. The ss.AAV.CMV.BAG3 cassette for gene transfer comprises the CMV enhancer/promoter, the full-length BAG3 cDNA, and the SV40 polyA tail and is provided as SEQ ID NO: 13.

### Generation of New Plasmids Suitable for Systemic Treatment of Protein Aggregate Myopathies and Neurodegenerative Disorders.

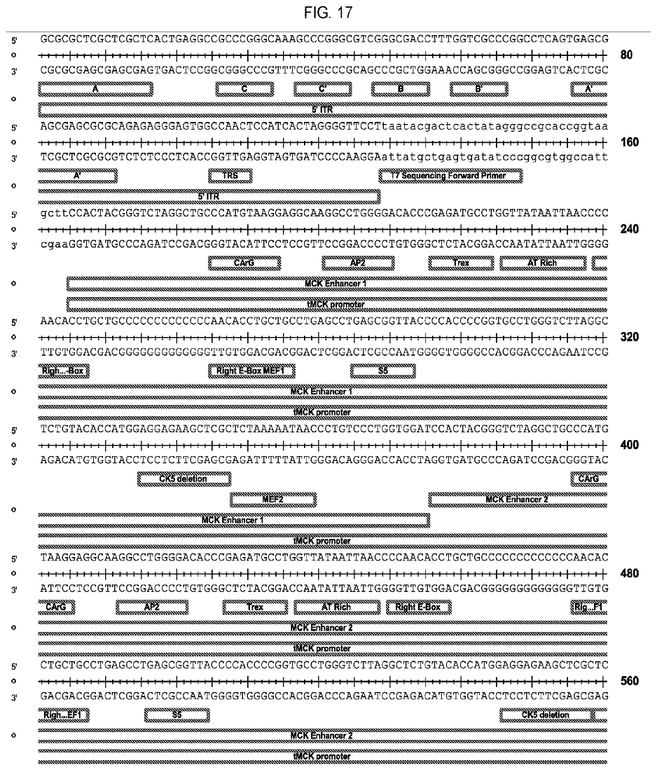

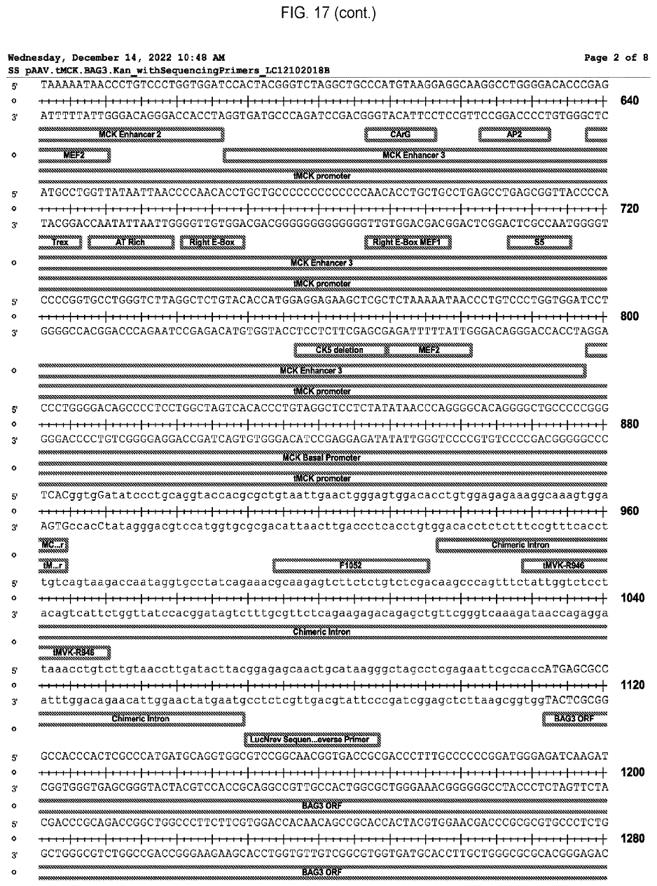

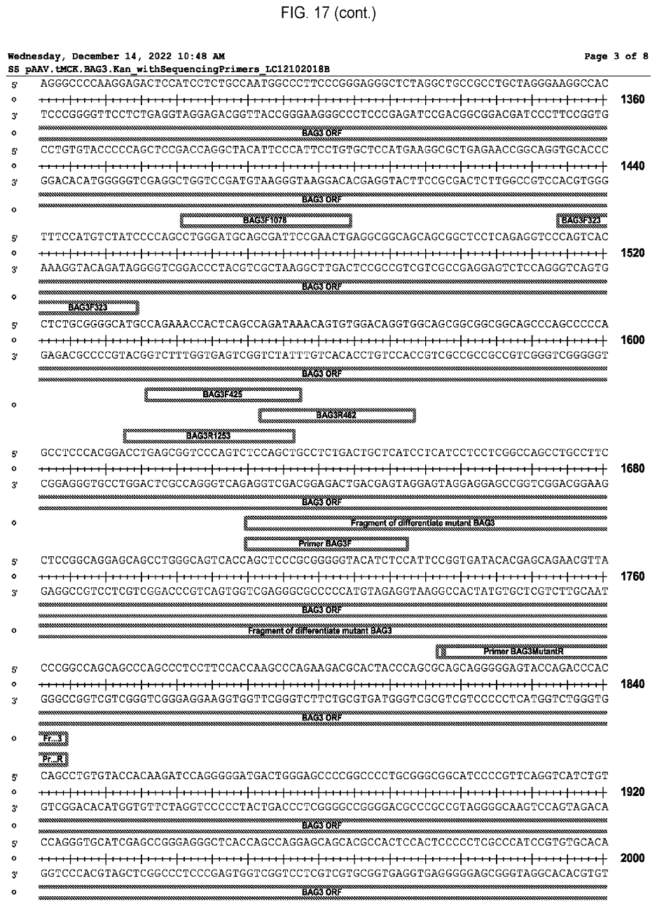

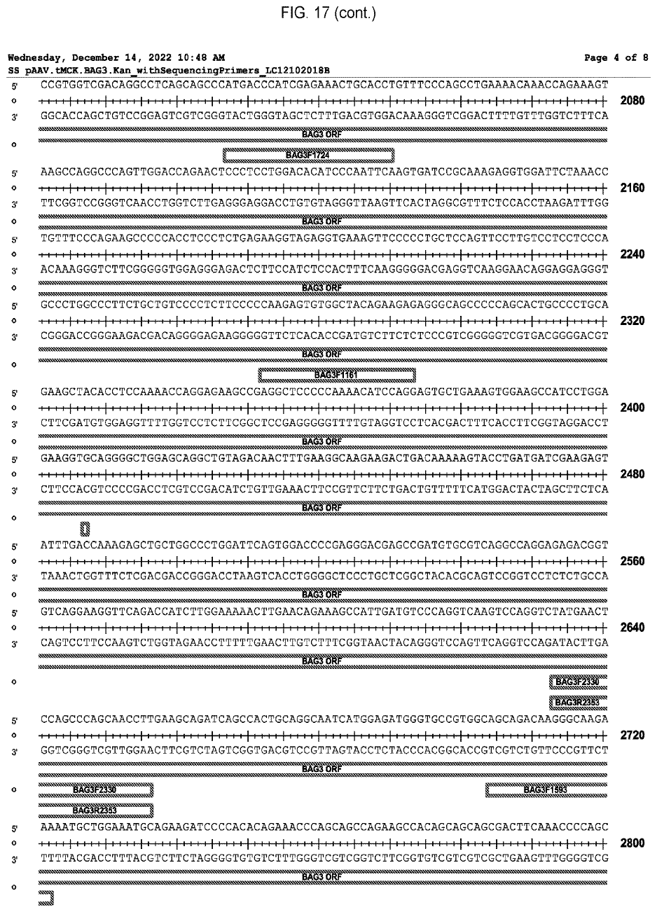

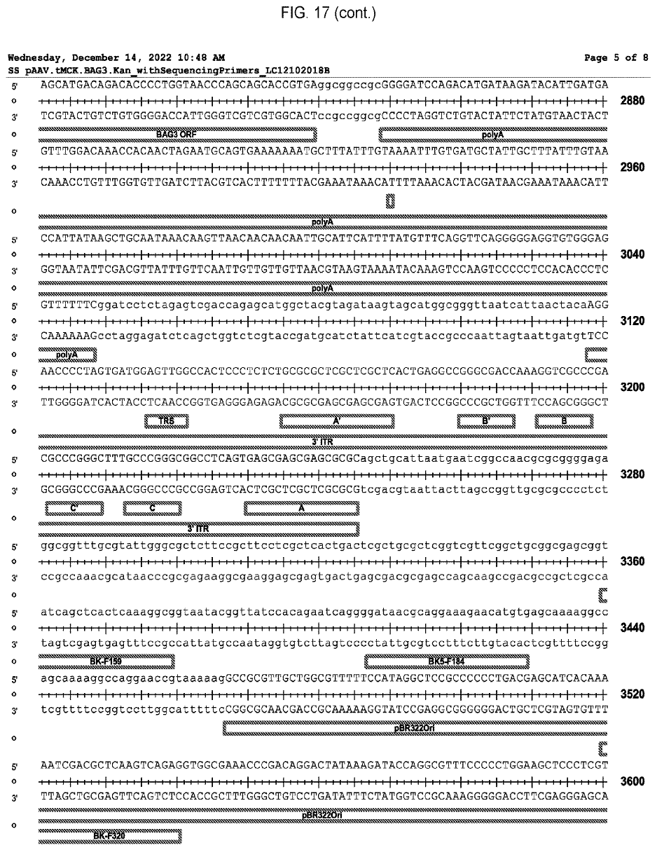

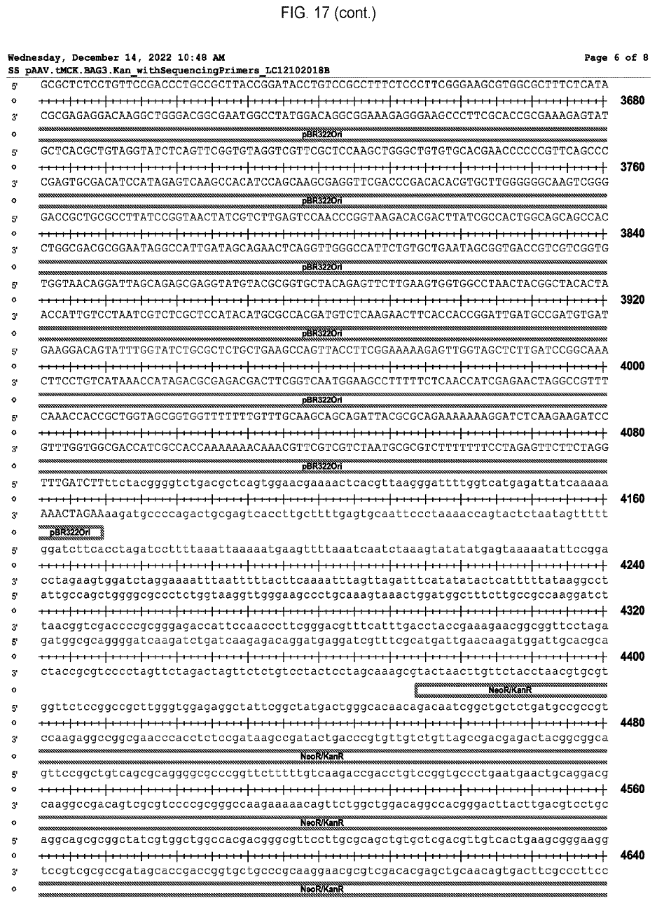

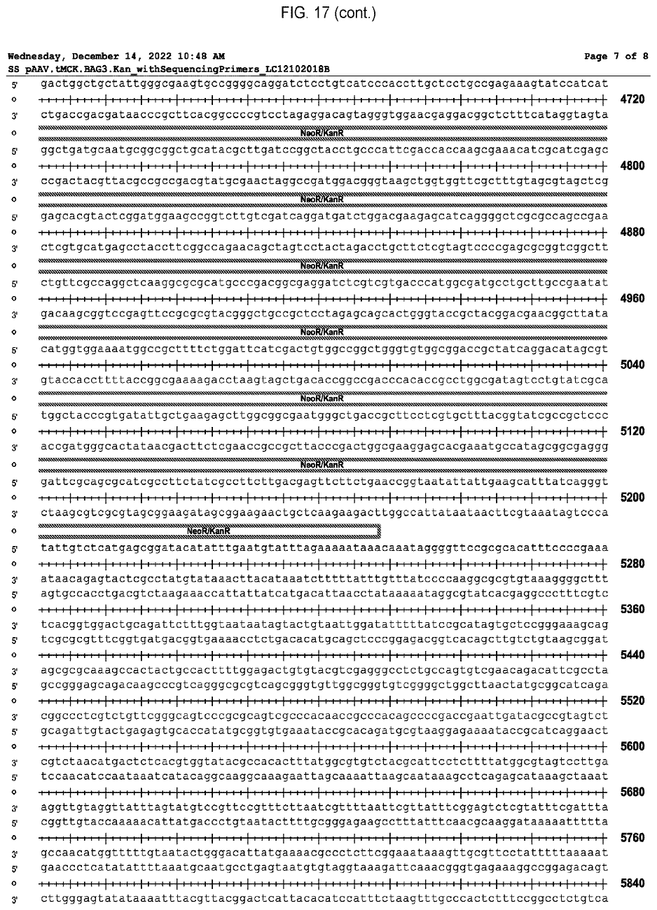

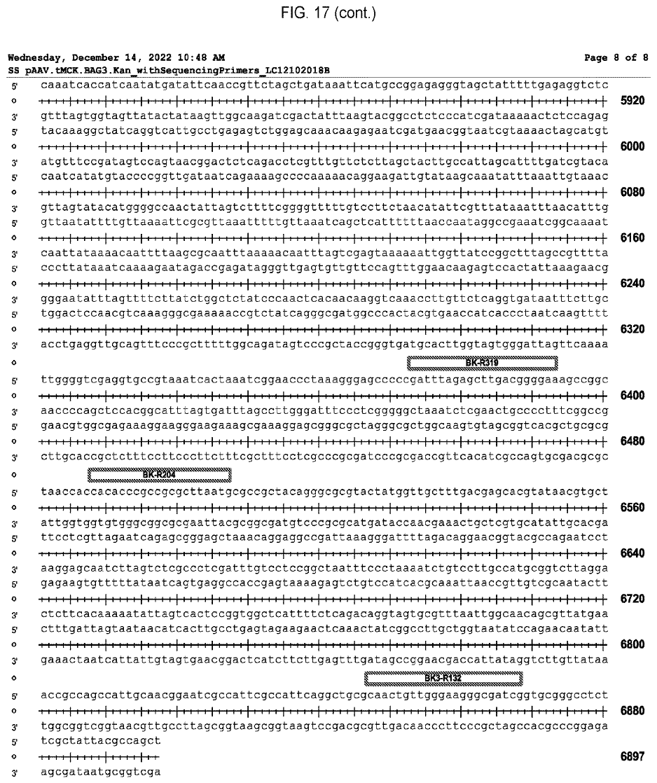

Additional plasmids were generated that were suitable for systemic treatment of protein aggregate myopathies and neurodegenerative disorders. The CMV promoter was replaced with a muscle specific promoter, tMCK enhancer/promoter, to avoid potential off-target effects and the serotype was switched to AAVrh74 serotype. The construct also comprised a SV40 polyA tail. The AAVrh74.tMCK.hBAG3 vector was generated for the treatment of animal models of myofibrillary myopathies (FIG. 1B). The sequence of AAVrh74.tMCK.hBAG3 is provided in FIG. 17 (SEQ ID NO: 1).

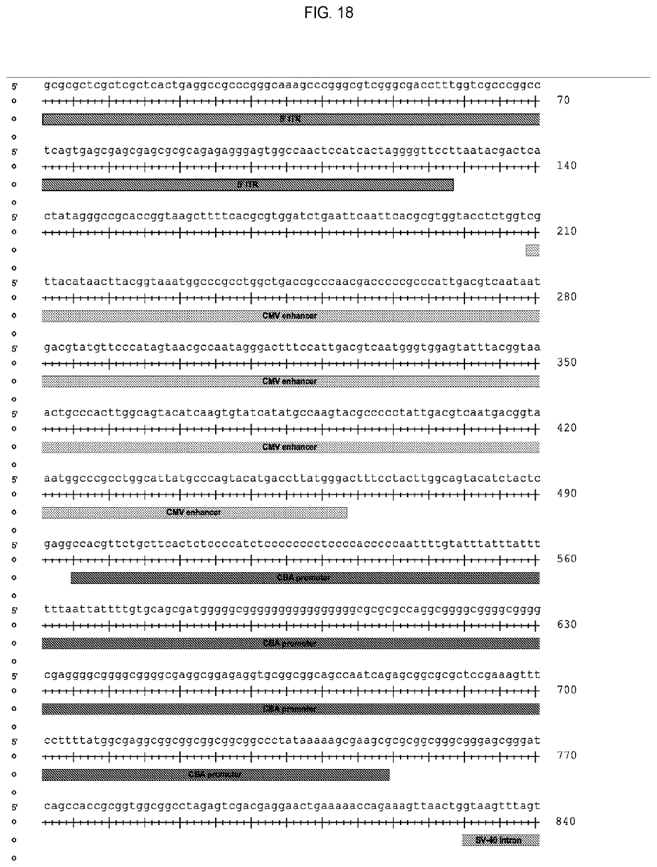

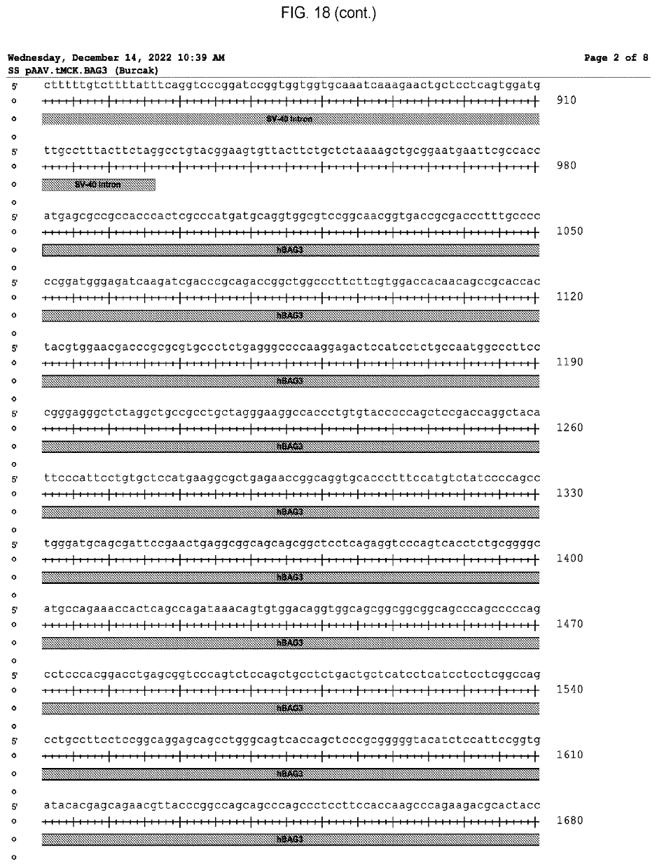

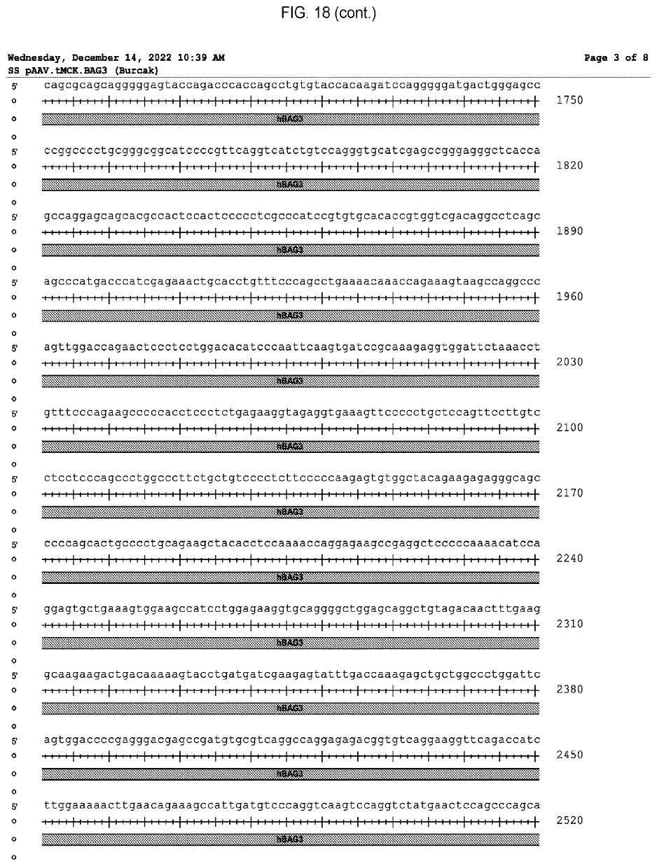

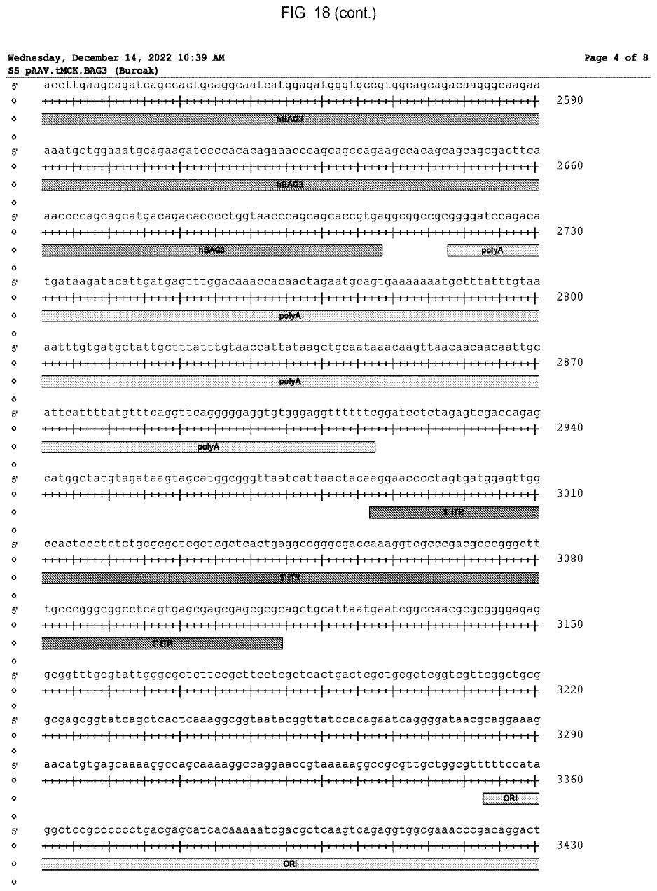

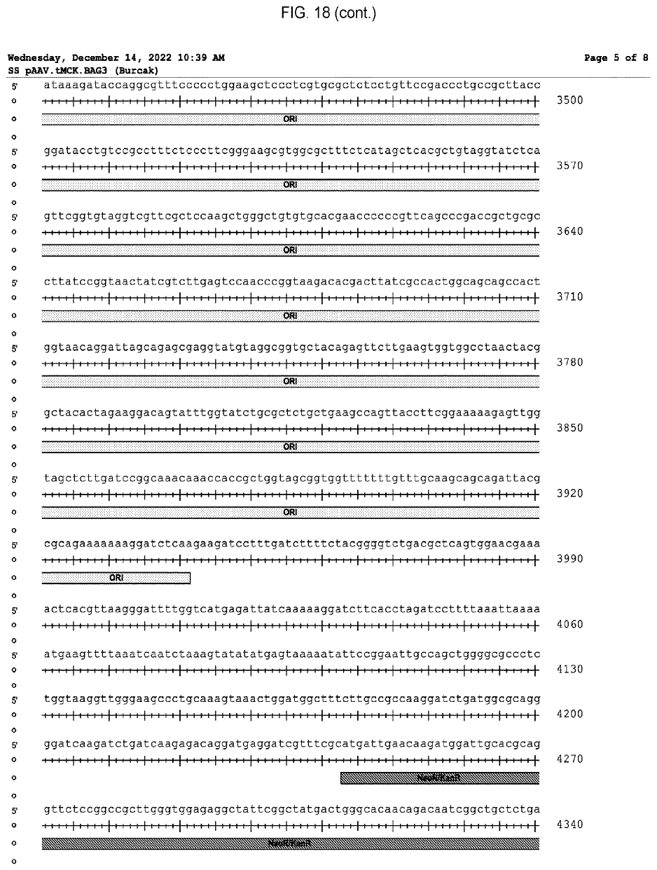

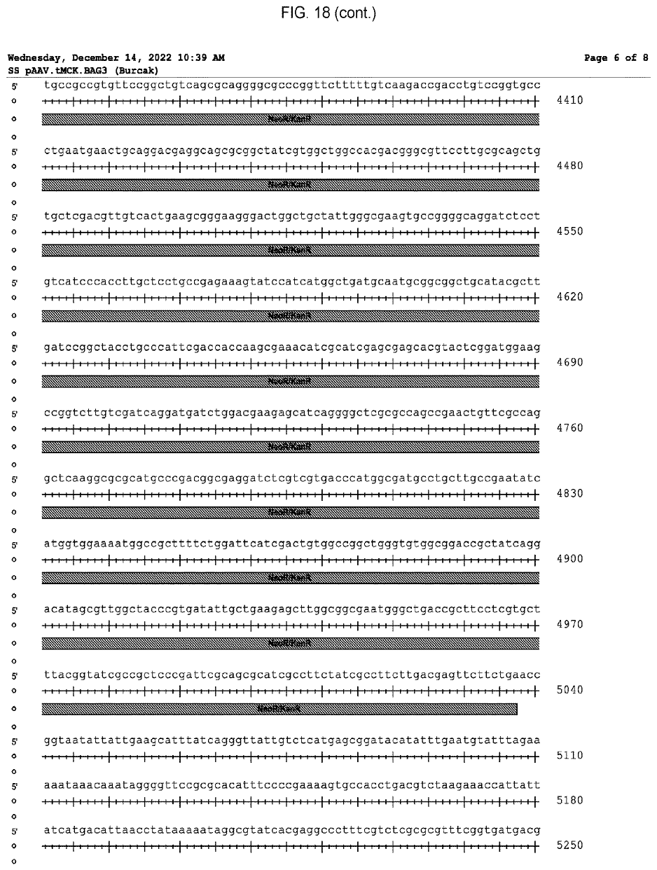

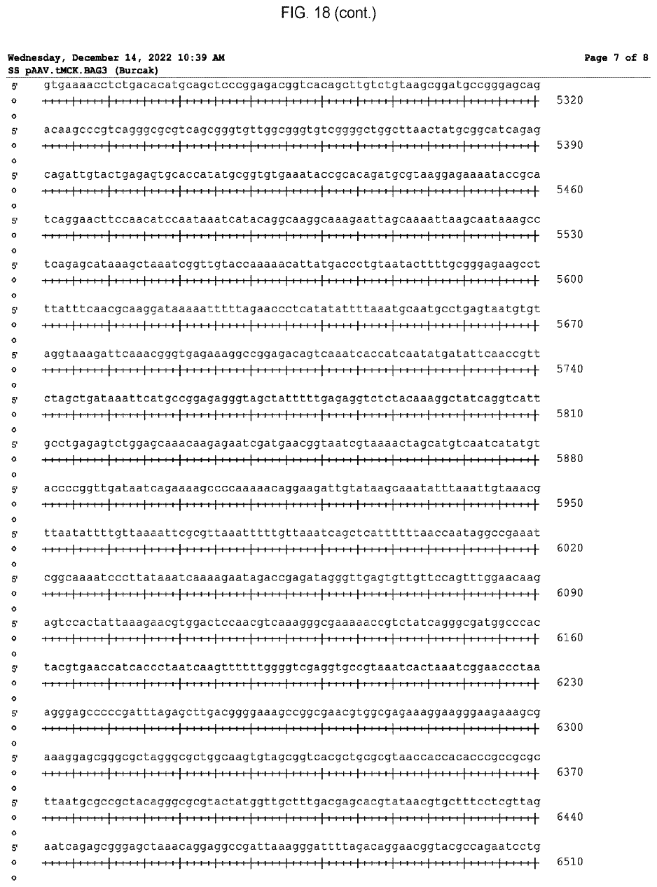

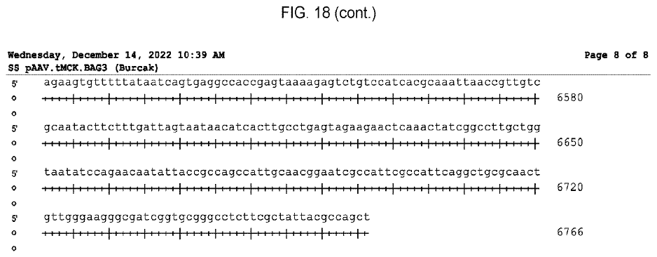

In addition, a plasmid was generated in which the CMV promoter in the initial cassette was replaced with the chicken beta actin (CBA) promoter/CMV enhancer, a SV40 tail was included and the serotype was switched to the AAV9 serotype. The AAV9.CBA.hBAG3 vector was generated to treat rodent models of misfolded protein related neurodegenerative diseases systemically (FIG. 1C). The sequence of AAV9.CBA.hBAG3 is provided in FIG. 18 (SEQ ID NO: 2).

rAAV vectors were produced by a modified cross-packaging approach whereby the Rep proteins from the AAV2 serotype are used to package vector genomes flanked by AAV2 ITRs into a capsid other than AAV2, in this case, AAVrh74, AAV1 or AAV9 capsids Rabinowitz et al., J Virol. 76 (2): 791-801 (2002). Production was accomplished using a standard three plasmid DNA/CaPO4 precipitation method using HEK293 cells. HEK293 cells were maintained in DMEM supplemented with 10% fetal bovine serum (FBS) and penicillin and streptomycin. The production plasmids were: (i) plasmids encoding the BAG3 therapeutic protein or NT-3 therapeutic protein, (ii) rep2-capX modified AAV helper plasmids encoding cap serotype isolate rh.74, and (iii) an adenovirus type 5 helper plasmid (pAdhelper) expressing adenovirus E2A, E4 ORF6, and VA I/II RNA genes. A quantitative PCR-based titration method was used to determine an encapsidated vector genome (vg) titer utilizing a Prism 7500 Taqman detector system (PE Applied Biosystems). [Clark et al., Hum Gene Ther. 10 (6): 1031-1039 (1999)]. A final titer (vg ml⁻¹) was determined by quantitative reverse transcriptase PCR using the specific primers and probes utilizing a Prism 7500 Real-time detector system (PE Applied Biosystems, Grand Island, NY, USA). Aliquoted viruses were kept at 80° C.

All plasmids used to make AAV genomes to be packaged also contain a Kanamycin (KanR) or Ampicillin (AmpR) resistance gene outside of the ITR sequences used for packaging of the genome. This allows for the DNA encoding the AAV genome to be transformed into bacteria to produce large amounts of DNA in the presence of either antibiotic, which kills all non-transformed bacteria. KanR and AmpR are not packaged into the AAV capsid in the AAV genome used to treat patients, but their presence allows for DNA production in bacteria.

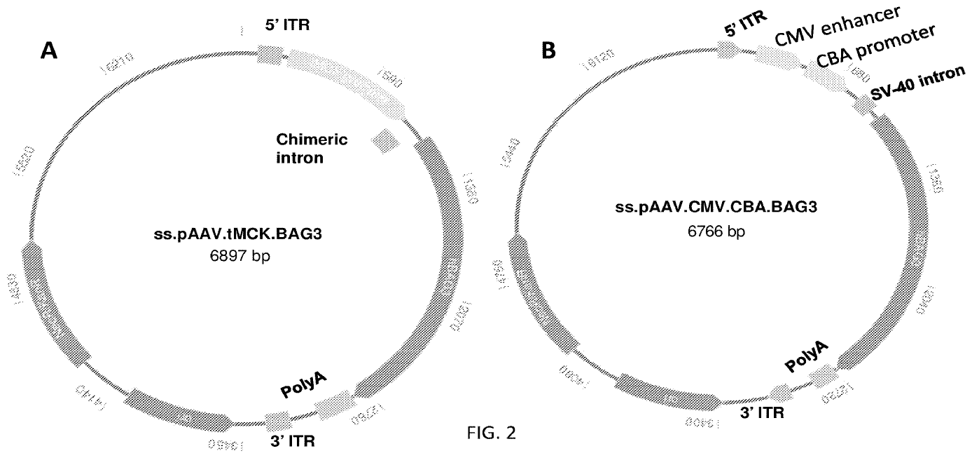

A description of ss.pAAV.tMCK.BAG3.Kan set out as SEQ ID NO: 1 is provided in Table 1 below. The plasmid map for this vector is provided in FIG. 2A.

| | Nucleotides in | | |
|---|---|---|---|
| Element | SEQ ID NO: 1 | Direction | Length |
| 5′ ITR | 1-128 | [symbol] | 128 nt. |
| tMCK promoter | 165-884 | [symbol] | 720 nt. |
| Chimeric intron | 937-1069 | [symbol] | 133 nt |
| Human BAG3 coding | 1112-2839 | [symbol] | 1728 nt. |
| sequence | | | |
| SV40 polyA | 2849-3048 | [symbol] | 200 nt. |
| 3′ITR | 3118-3245 | [symbol] | 128 nt |

A description of ss.pAAV.CMV.CBA.BAG3, set out as SEQ ID NO: 2, is provided in Table 2 below. The plasmid map for this vector is provided in FIG. 2B.

| | Nucleotides in | | |
|---|---|---|---|
| Element | SEQ ID NO: 2 | Direction | Length |
| 5′ ITR | 1-128 | [symbol] | 128 nt. |
| CMV enhancer | 209-463 | [symbol] | 255 nt. |
| CBA promoter | 495-749 | [symbol] | 255 nt. |
| SV-40 intron | 830-926 | [symbol] | 97 nt. |
| Human BAG3 coding | 981-2708 | [symbol] | 1728 nt. |
| sequence | | | |
| polyA | 2718-2917 | [symbol] | 200 nt. |
| 3′ ITR | 2987-3114 | [symbol] | 128 nt |

A description of ss.pAAV.CMV.hBAG3, set out as SEQ ID NO: 13, is provided in Table 3 below.

| | Nucleotides in | | |
|---|---|---|---|
| Element | SEQ ID NO: 13 | Direction | Length |
| 5′ ITR | 1-128 | [symbol] | 128 nt |
| CMV promoter | 181-973 | [symbol] | 793 nt |
| CMV minimal promoter | 689-777 | [symbol] | 89 nt |
| Human BAG3 coding | 986-2713 | [symbol] | 1728 nt |
| sequence | | | |
| polyA | 2723-2922 | [symbol] | 200 nt |
| 3′ ITR | 2922-31119 | [symbol] | 128 nt |

Purification of AAV Particles by Density Gradient Centrifugation with Iodixanol

AAV particles were purified by iodixanol gradient centrifugation. The virus-containing cell lysate was clarified by centrifugation (3700 g, 4° C., 20 min) and the clarified lysate was transferred to a Quick Seal ultracentrifuge tube (26×77 mm, Beckman). Different concentrations of iodixanol solution (Sigma) were layered under the virus-containing lysate. This created an iodixanol gradient consisting of 60% 6.0 ml, 40% 5.0 ml, 25% 6.0 ml, and 15% 9.0 ml iodixanol with the virus solution at the top. The gradient was centrifuged in an ultracentrifuge for 1 hour at 416,000 g at 18° C. Next, a 40% phase containing AAV particles was extracted using a cannula by piercing the tube at the bottom of the 40% phase and dropping the solution into the collection tube until the 25% phase was reached. The 40% phase AAV capsid titer was determined using a commercially available ELISA (AAV titer ELISA, Progen).

### Example 2

### Construction of NT-3 Expressing AAV Construct

Design of self-complementary rAAV viral vectors with serotype 1 containing NTF3 cDNA under tMCK was described previously in Sahenk et al., Mol Ther, 22 (3): 511-521 (2014), which is incorporated by reference herein in its entirety. Aliquoted viruses were kept in −80° C. until use. Blood samples were collected from treated and non-treated mice by eye bleeding under anesthesia at 6 and 16 weeks post injection and serum was assayed for NT-3 levels using a capture ELISA. The construct is referred to herein as scAAV1.tMCK.NTF3.

A tMCK promoter/enhancer sequence was used to drive muscle-specific gene expression and is composed of the muscle creatine kinase promoter with an added enhancer element (enh358MCK, 584-bp) fused to it. A triple tandem of the MCK enhancer (206-bp) was ligated to the 87-bp basal promoter in the tMCK promoter/enhancer.

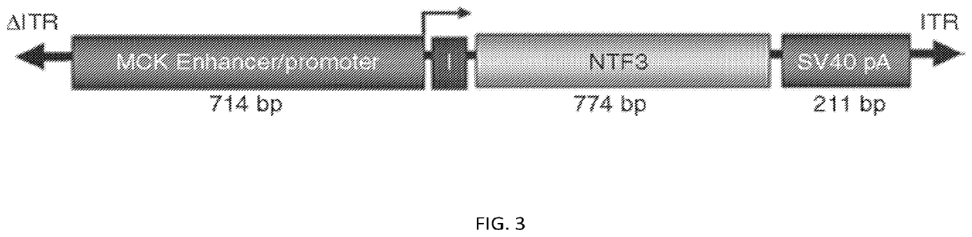

The scAAV1.tMCK.NTF3 drug product was produced by 3 plasmid DNA transfection of human HEK293 Master Cell Bank cells with: (i) the pAAV.tMCK.NTF3-vector plasmid (see FIG. 3), (ii) an AAV1 helper plasmid termed R88/C1 containing the AAV rep2 and Cap1 wild-type genes and (iii) the helper adenovirus plasmid.

A schematic representation of the plasmid with molecular features and open reading frames is shown in FIG. 3. The rAAV genome derived from pAAV.tMCK.NTF3 plasmid is a self-complementary DNA genome containing the human NTF3 cDNA expression cassette flanked by AAV2 inverted terminal repeat sequences (ITR). It is this sequence that is encapsulated into AAV1 virions. Plasmid pAAV.tMCK.NTF3 was constructed by inserting the tMCK expression cassette driving a NTF3 gene sequence into the AAV cloning vector psub201. The human NTF3 gene is expressed from the mouse triple tandem MCK promoter which is a modification of the previously described CK6 promoter and contains a triple E box sequence. An SV40 polyadenylation signal is used for efficient transcription termination. The cassette also contains a chimeric intron for increased gene expression and is composed of the 5′ donor site from the first intron of the human β-globin gene and the branchpoint and 3′ splice acceptor site from the intron that is between the leader and the body of an immunoglobulin gene heavy chain variable region. The NTF3 expression cassette has a consensus Kozak immediately in front of the ATG start and 200 bp SV40 polyA signal for efficient mRNA termination. The NTF3 cDNA is included in its entirety (NCBI Reference Sequence: NM_001102654). The only viral sequences included in this vector are the inverted terminal repeats of AAV2, which are required for both viral DNA replication and packaging. The AAV ITRs are sequences that are nearly identical on both ends, but in opposite orientation. The “left” (mutated) ITR has the terminal resolution site deleted to allow hairpin formation of the genome. The identities of all DNA plasmid elements are confirmed by DNA plasmid sequencing on the plasmid source stock.

Shown in Table 4 are the base pair locations of relevant molecular features within the rAAV vector DNA plasmid of SEQ ID NO: 12.

TABLE 4
Molecular Features of plasmid sc pAAV.tMCK.NTF3

| TYPE | START | END | NAME | DESCRIPTION |
|---|---|---|---|---|
| REGION | 7 | 112 | 5′ ITR | AAV2 inverted |
| | | | | terminal repeat with |
| | | | | terminal resolution |
| | | | | site deleted |
| REGION | 147 | 860 | tMCK | Mouse muscle |
| | | | promoter/ | creatine kinase |
| | | | enhancer | promoter/enhancer |
| REGION | 892 | 1024 | chimeric | 5′ donor site from |
| | | | Intron | human β-globin and |
| | | | | the branchpoint and |
| | | | | 3′ splice acceptor |
| | | | | site from IgG HC |
| | | | | variable region. |
| GENE | 1077 | 1850 | Human | Human NTF3 gene |
| | | | NTF3 gene | |
| REGION | 1860 | 2059 | SV40 pA | SV40 polyadenylation |
| | | | | signal |
| REGION | 2121 | 2248 | 3′ ITR | Wild-type AAV2 |
| | | | | inverted terminal |
| | | | | repeat |
| GENE | 4032 | 4892 | AmpP^{r} | AmpP^{r}P resistance gene |
| REGION | 5040 | 5707 | ori | Plasmid origin of |
| | | | | replication |

### Example 3

### Mouse Models

### The Rodent Model Expressing Myotilin T57I Mutation

Myotilin T57I transgenic mice we used in our studies, models the autosomal dominant LGMD1A by co-expressing a mutant human myotilin transgene in the presence of normal levels of endogenous WT murine myotilin28. TgT57I levels are 2.6-fold higher than endogenous levels. Immunostaining of frozen muscle cross-sections shows that transgene expression is uniform across myofibers within a given muscle group and also similar both in slow type I and fast type II fibers. Muscle pathology is progressive, with both the size and number of aggregates increasing with age starting at 2 weeks of age as small focal points and subsequently reaching up to 40 μm in older mice. Immunostaining has shown that the aggregates in TgT57I muscle harbor several Z-disc proteins (ACTN2, FLNC and desmin); sarcomeric proteins titin and myosin localize to the aggregates as well. In addition, the aggregates contain ubiquitinated protein.

TgT57I mice show contractile dysfunction in the extensor digitalis longus muscle tested at 8 months of age; muscle mass and muscle fiber cross sectional area are reduced as well. The soleus and diaphragm muscles, however, are completely spared of any pathology or physiological deficits28.

### SOD1-G93A Mouse Model for ALS

Amyotrophic Lateral Sclerosis (ALS or Lou Gehrig's disease) is a neurodegenerative disease characterized by degeneration of upper- and lower-motor neurons leading to progressive muscle atrophy and weakness, and ultimately to paralysis. The onset of ALS typically occurs between the ages of 55-75 and death, often resulting from respiratory failure, usually occurs within 3 years from disease onset for 50% of the patients. While most ALS cases (90%) have no clear genetic basis (sporadic ALS), approximately 10% of ALS is familial (familial ALS), inherited usually in an autosomal dominant fashion and caused by mutations in multiple genes. However, both forms of the disease are molecularly and clinically indistinguishable (Bosco and Landers, 2010; Gros-Louis et al., 2006). Currently, more than 20 genes have been associated with fALS, of which four account for the majority of familial cases which include: the Chromosome 9 Open Reading Frame 72 gene (C9ORF72, 40%), Superoxide Dismutase 1 (SOD1, 20%), Fused in Sarcoma (FUS, 1-5%), and TAR DNA Binding Protein (TARDBP, 1-5%).

Although the etiology of ALS remains poorly understood, abnormal protein aggregation and altered proteostasis are common features of sporadic and familial ALS forms. In familial ALS, several genes that are mutated lead to the formation of abnormally folded proteins which aggregate and form inclusion bodies as well as actively impair proteostasis mechanisms Soo et al. 2015. Acta Neuropathol. 130, 679-97). These are mainly SOD1, TAR DNA binding protein 43 (TDP-43), FUS, and C9ORF72 (Andersen P M, Al-Chalabi A., 2011. Nat Rev Neurol. 2011; 7 (11): 603-615; DeJesus-Hernandez et al., 2011 Neuron. 72 (2): 245-256; Guo et al. 2011. Nat Struct Mol Biol. 18:822-830; Guo et al. 2010. Brain Res. 1353, 234-44). Furthermore, dominant mutations in genes encoding for components of the protein quality control machinery related to vesicle transport, autophagy, ER homeostasis and UPS such as tubulin 4A (TUB4A), Dynein, Dynactin, sequestosome-1 (p62), Optineurin, valosin-containing protein (VCP or p97), vesicle-associated membrane protein associated protein B (VAPB), TANK1 binding kinase 1 (TBK1) and Ubiquilin-2 are all associated with ALS (Deng et al., 2011; Fecto et al. 2011. Arch Neurol. 68, 1440-6; Freischmidt et al. 2015. Nat Neurosci. 18, 631-6.; Hipp et al. 2014 Trends in Cell Biology. 24, 506-514; Johnson et al. 2010. Neuron. 68, 857-64; Rademakers and van Blitterswijk, 2014; Smith et al. 2014. Neuron. 84, 324-31; Synofzik et al., 2012; Williams et al. 2012. Neurobiol Aging. 33, 2527 e3-10). Moreover, intracellular proteinaceous inclusions are a hallmark neuropathological feature of ALS. The majority of ALS patients present with inclusions in both degenerating neurons and surrounding glia (Wood et al., 2003 Neuropathology and Applied Neurobiology. Vol. 29, ed., pp. 529-545; Piao et al. 2003. Brain Pathol. 13, 10-22; Nishihira et al., 2008. Acta Neuropathol. 116, 169-182; Zhang et al. 2008. Acta Neuropathol. 115, 115-122.) in the brainstem and spinal cord, but also in the cerebellum, hippocampus, and the frontal and temporal lobes (reviewed in Al-Chalabi et al. 2012. Acta Neuropathol. 124 (3): 339-352). The most common inclusions are of ubiquitinated proteins, which are found in both the upper and lower motor neurons (Neumann et al. 2006. Science 314, 130-133.), and are suggestive of defects in protein turnover (Blokhuis et al. 2013. Acta Neuropathol. 125 777-794).

Preclinical ALS research requires animal models that are based on the same genetic alterations as observed in patients and in parallel closely mimic the disease pathology. The association of the aforementioned genes with ALS has prompted the generation of transgenic animal models expressing ALS-linked mutations, primarily to understand the pathological mechanisms leading to neuronal death, and to develop therapeutic strategies. In 1993, SOD1 became the first gene to be linked to ALS (Rosen et al. Nature. 1993; 362 (6415): 59-62; Dend et al. Science. 1993; 261 (5124): 1047-1051). SOD1 encodes a cytosolic Cu/Zn superoxide dismutase that catalyzes the dismutation of toxic superoxide anion to oxygen and hydrogen peroxide. Mutations in SOD1 account for ~20% of fALS cases. The first mouse model of ALS was developed after the identification of mutations in the SOD1 gene. A transgenic mouse overexpressing mutant (G93A) SOD1 was created by insertion of multiple copies of the mutated human SOD1, which are randomly inserted into chromosome 12 of the mouse genome (Gurney et al. 1994. Science 264, 1772-1775; Achilli et al. 2005 Amyotroph. Lateral Scler. Other Motor Neuron Disord. 6, 111-114). The SOD1-G93A mice develop phenotypic and pathological symptoms that recapitulate hallmark signs of ALS in patients ALS in humans (Gurney et al. 1994. Science 264, 1772-1775) including the progressive development of muscle weakness leading to paralysis (Kong, J and Xu, Z. 1998. J. Neurosci. 18, 3241-3250). The moment of disease onset and the life span of these mice are related to the level of overexpression of mutant SOD1, while overexpression of nonmutated SOD1 gives no phenotype. The SOD1-G93A mice develop adult-onset (at ~90 days) neurodegeneration of spinal motor neurons and progressive motor deficits leading to paralysis in one or more limbs within a few weeks of age, which compares well with human ALS pathology (Synofzik et al., 2010. J Neurol Neurosurg Psychiatry. 81 (7): 764-767). Moreover, intracellular protein aggregates, an important feature evident in the motor neurons of ALS patients, are also evident in the mutant SOD1 mice (Bruijn et al. Science. 1998; 281 (5384): 1851-1854; Watanabe et al Neurobiol Dis. 2001; 8 (6): 933-941), represent an important feature of ALS. The accumulation of misfolded and aggregation-prone protein suggests an imbalance of protein homeostasis (proteostasis). The rate of disease progression and death in SOD1-G93A mice can however be slower or faster depending on the genetic background in which the mutant SOD1 is expressed. The B6/SJL mixed strain have an average lifespan of 129 days, while a B6 background strain lives around 144-161 days, a SJL strain lives around 119 days, and the 129 strain lives around 125 days (10, 11). Additionally, copy number variation of the mutant SOD1 transgene can alter the severity of the phenotype (Acevedo-Arozena et al. 2011. Dis Model Mech. 4 (5): 686-700).

Since its development in 1994, SOD1G93A mouse remains the most commonly used ALS animal model, and accordingly most of our knowledge of the etiology and pathogenesis of the disease comes from studies carried out using this animal model. This model is also freely available adding to its popularity.

At present, it is still unclear how SOD1 mutations selectively cause motor neuron death. Studies using the SOD1G93A transgenic mice and SOD1 knockout mice suggest that the motoneuron degeneration does not result from a loss of SOD1 enzyme activity but from a toxic effect induced by the presence of mutant SOD1 itself (“toxic gain-of-function”) (Gurney et al. 1994; Reaume et al. 1996; Wong et al. 2002). However, the accumulation of misfolded and aggregation-prone protein suggests an imbalance of protein homeostasis (proteostasis). Molecular chaperones are critical factors for maintaining proteostasis through facilitating protein folding and quality control. In fact, In mutant SOD1 (mutSOD1) ALS models, aggregation correlates to impaired functions of proteasome and/or autophagy, both essential for the intracellular chaperone-mediated protein quality control (PQC), and to a reduced mutSOD1 clearance from motoneurons. Indeed, the heat shock response co-inducer arimoclomol, which upregulates molecular chaperone expression, can protect against mutant SOD1 toxicity in vivo.

The studies described herein tested whether the overexpression of the co-chaperone, BAG3 has beneficial effects in the SOD1-G93A mouse models of ALS e.g., reduction of mutant SOD1 aggregation, improve viability in cells and/or alleviate motoneuron degeneration.

### Source of Mice

MYOT (TgT57I), C57BL/6 wild type (WT) and, SOD1-G93A (B6SJL-Tg(SOD1*G93A)1Gur/J, Strain #: 002726) mice were obtained from Jackson Laboratory (Bar Harbor, ME). Hemizygous SOD1-G93A mice were bred with C57BL/6 wild type mice. To minimize the animal variability due to sex differences, hemizygous SOD1-G93A female mice were included in the experiments. All animal experiments were performed according to the guidelines approved by The Research Institute at Nationwide Children's Hospital Animal Care and Use Committee.

### Example 4

### Materials and Methods

### Cell Lines

The HEK293 cell line, which is derived from immortalized human embryonic kidney cells and the was purchased from ATCC. The WP-4 murine tumor cell line, which is a clone of the methylcholanthrene-induced murine fibrosarcoma cell line MCA205 was obtained from Agonox (Wexler et al., J. Natl. Cane. Inst., Vol. 63, pg. 1393, 1979; Asher et al., J. Inmunol, Vol. 146, pg. 3227, 1991; Mule et al, Hum Gene Ther. 7 (13): 1545-53, 1996).

### Cell Culture

HEK293 cells were maintained as a monolayer in Dulbecco's modified Eagle's (DMEM) medium (Corning no. 10013CV or alternative) supplemented with 10% heat-inactivated fetal bovine serum (FBS) (Life Technologies no. 10437-028 or alternative) and 1% penicillin/streptomycin antibiotic solution (10 000 U/mL; Life Technologies no. 15140-122). WP-4 tumor cells were cultured in C10 culture medium (Roswell Park Memorial Institute (RPMI) 1640 growth medium (Gibco Life Technologies no. 11875-093) containing 2 mM L-glutamine (MilliporeSigma no. TMS-002-C), 1% penicillin/streptomycin (10 000 U/mL; Life Technologies), 1× non-essential amino acids (Gibco no. 11140050), 1 mM sodium pyruvate (Gibco no. 11360070), 10 mM HEPES buffer (ThermoFisher no. 15630-080), 1×β-mercaptoethanol (Sigma-Aldrich no. ES-007-E), and 10% FBS (Life Technologies no. 10437-028 or alternative)). Cell lines were genetically authenticated using STR-based DNA profiling and multiplex PCR and confirmed to be mycoplasma and endotoxin free using the MycoAlert Detection kit (Lonza) and the Endosafe-PTS system (Charles River Laboratories). Cells were cultured at 37° C., 5% CO₂ in a humidified incubator.

### WP-4 Cells Electroporation and Transfection

rAAV Vector Infection In Vitro

WP-4 cells were seeded on 6 cm diameter dishes at the density of 5×10⁴. Cells were allowed to attach for 24 h. After 24 h, normal complete media was changed to RPMI 1640 with reduced, 2% FBS. rAAV vectors were moved from −80° C. to room temperature, and underwent a gentle vortex and a short spin. rAAV suspension was prepared using buffer (PBS) only and this was then added directly to the cells at a multiplicity of infection (MOI) of 40,000 genome copies or 0 genome copies (non-transduction control group). After incubation with virus for 2 h, media were replaced to remove vectors that did not transduce. Thereafter, spent media was replaced every 48 h. Seven days later, the efficiency of cell transduction was evaluated using several methods. Cells stably expressing the gene of interest were selected by treatment with 1 to 3 μg/ml puromycin over 2 days (InvivoGen).

The plates were examined for GFP-positive cells by fluorescence microscopy and the number of positive cells was calculated.

### AAV Vector Delivery to Mice

### Intramuscular (IM) Injection of the TA of Mouse

Three to four-week-old mdx mice and normal age-matched C57/BL10 were used for IM injection. Mice were anesthetized and maintained on 1-4% Isoflurane (in oxygen). Both hindlimbs were shaved, and the TA muscle was injected with 3×1010 vg of rAAV8.micro-dystrophin.FLAG or normal saline (30 μl volume) using a 30-gauge insulin syringe.

### Intramuscular (IM) AAV Delivery

IM injections were given in the tibialis anterior muscle of wild-type (C57BL/6J) or TgT57I transgenic (MYOT) mice by direct injection in a volume of 50 μL of PBS, with control mice given PBS alone in an identical volume. Doses of ssAAV9.CMV.hBAG3 given were in a total of 2.5×10¹¹ vg. Alternatively, the same MYOT mouse was used with the left GAS muscle being injected and the right GAS muscle serving as the control. Titers for IM injections were measured using a linear DNA standard. Injected mice exhibited no abnormal clinical signs following any of the injections.

For IM safety experiments with ssAAVrh74.tMCK.hBAG3, doses of 1.0×10¹¹ vg, 4.0×10¹¹ vg, 8.0×10¹¹ vg were injected intramuscularly into wild-type (C57BL/6J) mice. For IM ssAAVrh74.tMCK.hBAG3 dose titration, a male only cohort of MYOT mice were injected with 3.0×10¹¹ vg, 1.0×10¹¹ vg, 2.0×10¹¹ vg.

For the ALS mouse model, SOD-G93A mice were injected with ssAAV9.CBA.hBAG3 at a dose of 3.0×10¹¹ vg and/or ssAAV1.tMCK.NT-3 at a dose of 1.0×10¹¹ vg.

### Systemic AAV Delivery

Mice were placed in a mouse tube restrainer (Braintree Scientific; Braintree, MA, USA) with the tail outside the tube. The injected region was prepped with povidone-iodine and 70% ethanol, after which AAV vector or AAV buffer was injected into the tail vein, usually in a volume of 150 UL and not exceeding a volume of 200 μL. For systemic dose titration, 3×10¹² vg or 6×10¹² vg doses of ssAAVrh74.tMCK.hBAG3 were injected into 12-16 week old MYOT mice. Titers for i.v. injections were measured using a linear DNA standard. Mice were sacrificed 8 weeks post injection and GAS, Quad, TA and Tri muscles harvested. The diaphragm was subjected to force measurements (see below), the FDB was tested for membrane repair ability and muscles and organs were harvested for expression and histopathology.

For efficacy assessment, ssAAVrh74.tMCK.hBAG3 at 3×10¹² vg was injected into the tail vein in 12-16 weeks old MYOT mice, n=3. Titers for i.v. injections were measured using a linear DNA standard.

### Immunofluorescence

Groups of mice were euthanized with an over-dosage of xylazine/ketamine anesthesia at 8 (n=2 in each cohort) and 12 weeks post gene injection (5 MYOT and 3 WT mice). GAS muscles harvested from 4- and 5-month-old male mice (i.e., 2 to 3 months post injection) were flash frozen in liquid nitrogen-chilled isopentane. Tissues were cryosectioned to a 12-μm thickness and mounted on Superfrost Plus microscope slides (Fisherbrand Superfrost Plus). The mounted tissue sections were fixed with 4% paraformaldehyde for 10 minutes at room temperature. Sections were washed with phosphate-buffered saline (PBS) containing 0.01% Triton X-100, permeabilized with 0.3% Triton X-100 for 5 minutes, and then incubated in a blocking buffer (1×PBS, 10% Goat Serum) for 1 hour at room temperature in a humidity chamber. The tissue sections were incubated at 4° C. overnight with Polyclonal Rabbit anti-Human MYOT primary antibody (1:400, LSBio Cat No. LS-C33471 or MyBioSource Cat No. MBS9127412). Following three, five-minute washes with a wash buffer (1×PBS, 1% Goat Serum), the tissue sections were incubated with AlexaFluor-594 conjugated goat anti-rabbit secondary antibody (1:500; Life Technologies, Grand Island, NY, USA; Cat No. A11032) for 1 hour at room temperature. Sections were washed with the wash buffer three times for 5 min and then counterstained with 0.5 mg/ml 4′,6-diamidino-2-phenylindole (DAPI, Sigma Cat No. D-9542) and cover-slipped using ProLong Gold Antifade Mountant (Molecular Probes Cat No. P36961). Image acquisition was performed with a using Nikon Ti2-E microscope (Nikon instruments, Tokyo, Japan) at ×10 magnification and analyzed using NIS-Elements (v5.3) software to determine aggregate size, fluorescent density of individual aggregates, and mean aggregate fluorescent density. Signal threshold was set with tissue sections stained with secondary antibodies alone to exclude nonspecific signal. Randomly selected representative 20×-magnification images were obtained using the Zeiss Axioskop microscope and Axiovision Rel software. Using the Bioquant software (Bioquant image analysis software, version 2016, R&M Biometrics Inc., Nashville, TN), red pixel counts were determined in each image and expressed as percent of the untreated control muscle in each mouse.

### H&E Staining

Muscle tissue was flash frozen in liquid nitrogen-chilled isopentane as described in the immunofluorescence section above. Sections of 12 μm were cut using a cryostat (Leica Biosystems, Wetzlar, Germany) and subjected to hematoxylin and eosin (H&E) as follows. Sections were fixed for 5 min, placed in 70% ethanol followed by rehydration in deionized water. Slides were stained in Harris modified hematoxylin solution (Sigma-Aldrich HHS32) for four and a half minutes, then washed in deionized water before being submerged in eosin Y solution (Sigma-Aldrich HT110116) for a minute and a half, and then washed again in deionized water until clear. The slides were dehydrated in an ascending alcohol series, cleared in xylene, and then mounted with cytoseal (Richard-Allan Scientific™ Cytoseal 60™ Thermo Scientific, 8310-16).

### Histological Analysis

The aggregate particle size and density were compared to untreated age-matched littermates. Twelve μm thick-cryostat sections were stained with H&E for routine histopathology, or immunostained with antibody against myotilin using standard protocols. Randomly selected representative 20×-magnification images were obtained in each biopsy using the Zeiss Axioskop microscope and Axiovision Rel software. 10× images covering the whole surface of the Immunostained sections were obtained using Nikon Ti2-E microscope and analyzed using NIS-Elements (v5.3) software to determine aggregate size, fluorescent density of individual aggregates, and mean aggregate fluorescent density. Myotilin aggregate burden was quantified using immunofluorescence technique on four randomly selected representative images at 20×-magnification from quadriceps muscles of BAG3-treated (n=10) and UT (n=8) cohorts. Aggregate density as number of aggregates/mm² and size distribution/mm² were calculated in treated and untreated cohorts.

### Western Blots

WP-4 cell lysates were prepared from in radioimmunoprecipitation assay (RIPA) buffer (Sigma) supplemented with a protease inhibitor cocktail (Roche Diagnostics) from 80% confluent tissue culture plates three days after transduction with ssAAV9.CMV.hBAG3. Serial cryosections of gastrocnemius muscles from treated and untreated mice or treated and untreated muscles from the same mouse were taken for protein preparation. Samples were harvested in buffer (125 mM Tris-HCl buffer [pH 6.8], 4% SDS, 5% glycerol, and 4 M urea) containing protease inhibitor (Roche Diagnostics). Insoluble material in the lysates was discarded following high-speed centrifugation at 4° C. Protein concentrations were determined using the Lowry assay (RC DC, Bio-Rad). Equal amounts of protein were loaded onto 3-8% TRIS-Acetate gradient gels (NuPAGE, Invitrogen) and transferred onto polyvinylidene difluoride (PVDF) membranes (Amersham Biosciences). Membranes were blocked with 5% nonfat dry milk in 1× Tris-buffered saline (TBS) containing 0.1% Tween-20 (TBST) at room temperature for 30 min. The membranes were then incubated with rabbit anti-BAG3 monoclonal (1:500, Abcam, Cat No. ab92309) primary antibody in blocking solution overnight at 4° C., followed by incubation with horseradish peroxidase-conjugated secondary antibody (0.02 μg/mL Goat Anti-Rabbit IgG-HRP, Vector Lab) at room temperature for 1 hour. After washing with TBST, blots were developed using enhanced chemiluminescence (ECL) reagent (Amersham Biosciences), and images were captured with a ChemiDoc (Bio-Rad Laboratories, Hercules, CA) or X-ray film exposure.

### Cresyl Violet Staining and Stereological Cell Counting

After euthanizing the mice and collecting the gastrocnemius muscles, the animals were transcardially perfused with normal saline followed by 10% formaldehyde for fixation. Spinal cords were removed, post-fixed in 4% PFA overnight at 4° C.

Cervical, thoracic, and lumbar segments of the spinal cord were paraffin-embedded, and transverse sections were cut for motor neuron count. For each segment, a total of seven 7-μm sections were cut at 49-μm intervals. For cresyl violet staining of paraffin embedded spinal cord sections, the tissues were deparaffinized in 2 or 3 changes of xylene of 3 minutes each followed by rehydration in 100% ethanol, twice for 3 minutes each. The tissues were then stained with 0.1% Cresyl violet for 4 minutes and quickly rinsed with tap water to remove excess stain. The tissues were washed with 70% ethanol followed by dehydration in two changes of 100% ethanol of 3 minutes each. The tissues were then cleared in xylene, mounted in Depex and air dried in the fume hood for 15-20 minutes.

Alternatively, the spinal cords were prepared from NaCl-perfused mice, separated in cervical, thoracic, and lumbar segments, and cryo-conserved. For motor neuron count, 12-μm cryosections, cut at 84 μm intervals, were fixed with acetone and subsequently stained with Cresyl violet. For cresyl violet staining of cryosections, the tissues were rehydrated in 95% ethanol, 75% ethanol, 70% ethanol, stained in cresyl violet for 30-45 sec while protecting the solution from light. The tissues were then dehydrated in 75% ethanol, 95% ethanol, 100% ethanol, 100% ethanol, 30 sec each. 0.1% Cresyl violet was prepared in water, filtered and stored at 4° C. for up to one month. After dehydration, sections were air-dried under a fume hood for 15-20 minutes and either imaged immediately or stored in a box with desiccant at −80° C. for up to several weeks.

20×-magnification Images of the spinal cord sections were obtained using Nikon Ti2-E microscope. Anterior horn cells in lumbar cord sections with distinguishable nucleolus were counted and the short axis through the center of nucleus was measured as diameter. All counted neurons, including interneurons with diameter<15 μm (presumed motor neurons, considered with diameter>15 μm) were located ventral to a drown straight line which is touching to the ventral border of the central canal and perpendicular to the hemi axis of spinal cord. Total number of neurons derived from both right and left anterior horn areas were included representing a screening of an approximately 350 μm thickness of the lumbar cord.

### Neurological Function Assessment

All measures of animal disease progression were performed blind by independent investigators to avoid subjective bias. Body weight was assessed weekly throughout the study. Nerve conduction studies, rotarod, and grip strength tests started on week 8 and were repeated weekly until 17 weeks of age.

Motor function testing/Grip strength:

Mice were tested for baseline motor function within 1 week prior to receiving i.m. injection of ssAAV1.CMV.NT-3 or PBS. Motor function tests included bilateral simultaneous hindlimb grip power and that of the left hind paw using a grip strength meter (Chatillon Digital Meter; Model DFIS-2; Columbus Instruments, Columbus, OH). Bilateral or unilateral grip strength was assessed by allowing the animals to grasp a platform then gently pulling them by the tail while a sensor recorded the force (Newtons, N) with which the mice resisted the pull before releasing the platform, as a measure of muscle strength. The force measurements were recorded in four separate trials. Measurements were performed on the same day and time of each week. Endpoint bilateral and ipsilateral grip strength measurements were done in two sessions (morning and afternoon), three trials in each per day for 3 consecutive days prior to obtaining the nerve conduction studies. The mean of these measurements were used to correlate with conduction studies.

### Rotarod Testing

Mouse motor function and balance was tested at baseline and endpoint by using the accelerating rotarod (Columbus Instruments, OH, USA). Mice were trained on the rotarod apparatus for 2 weeks to acclimate to testing protocol prior to data collection. The protocol was run at 5 rpm with a constant acceleration of 0.5 rpm/s, and the average of the best two out of three trials was included in the analysis.

### Nerve Conduction Studies

The animals were anaesthetized under 2% isoflurane and heating pad was set to 37 degrees Celsius to maintain body temperature. Right sciatic nerve conduction studies were performed using a Nicolet Viasys Viking Select EMG EP System (Nicolet Biomedical, Wisconsin, USA) and 27G disposable subdermal needle electrodes for both stimulation and recording as described previously (Pollari et al. 2018 J Vis Exp. (136): 57741). The stimulating electrodes were placed subcutaneously proximal (stimulus 1) and distal side (stimulus 2) of the sciatic notch with a distance of ~2 cm between the electrodes. The recording electrode was placed subcutaneously aligned with the long axis of gastrocnemius muscle and a reference electrode was inserted subcutaneously next to the Achilles tendon at a 30-degree angle, leaving 2-5 mm of the needle under the skin. The parameters measured included compound muscle action potential (CMAP) amplitude, distal latency, area, duration, and nerve conduction velocity. The distance between the two stimulation sites was used to determine the nerve conduction velocity.

Sciatic motor nerve conduction studies were performed bilaterally in each cohort mice at baseline and at 16 weeks after the onset of treatments using an electrodiagnostic system (Synergy N2 electromyograph; Natus, Middletown, WI) as has been previously reported (Yalvac et al., 2015, 2014). Briefly, the sciatic motor nerve conduction responses were recorded using two fine ring electrodes (Alpine Biomed, Skovlunde, Denmark) used as the active (E1) and reference (E2) electrodes. The active recording electrode was placed over the proximal portion of the gastrocnemius muscle and the reference electrode over the mid-metatarsal region of the foot. Using an irrigating syringe, the ring electrodes were precisely coated with electrode gel (Spectra 360 by Parker laboratories, Fairfield, NJ) to reduced skin impedance. A pair of 28 gauge monopolar needle electromyography electrodes (Teca, Oxford Instruments Medical, New York, NY) was used to deliver supramaximal stimulus to the sciatic nerve at the distal thigh and sciatic notch. The parameters measured included compound muscle action potential (CMAP) amplitude, distal latency, and conduction velocity.

### Example 5

### BAG3 Transduction Efficiency In Vitro and In Vivo

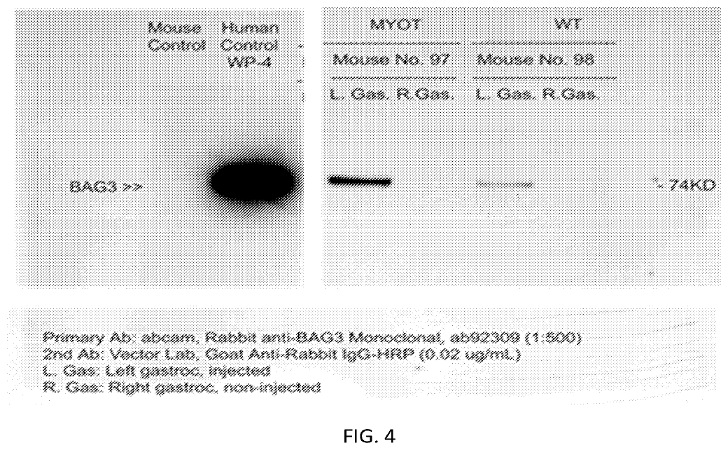

To test the transduction efficiency of the rAAV-mediated expression of BAG3 in vitro, WP-4 tumor cells were transduced with ssAAV9.CMV.hBAG3 and the transgene hBAG3 expression was detected in the lysates using a monoclonal antibody against hBAG3. Expression of the hBAG3 transgene was detected in WP-4 cell lysates using a monoclonal antibody against hBAG3 (FIG. 4, left panel).

A TgT57I transgenic (MYOT) mouse (No. 97) and a wild type (WT) mouse were used to test the biopotency of ssAAV9.CMV.hBAG3 in vivo. TgT57I mice recapitulate the progressive MYOT protein aggregation defects that characterize LGMD1A. In 3-month-old TgT57I mice, aggregates are associated with additional generalized muscle pathology, including deficits in myofiber size and gastrocnemius muscle weight, as well as slight but significant increase in myofibers with centrally located nuclei, which is a histological indicator that muscles underwent degeneration and were subsequently repaired. Importantly, these phenotypes are useful outcome measures for gene therapy. The effects of ssAAV9.CMV.hBAG3-mediated MYOT degradation was therefore examined on aggregate formation, myofiber diameter, muscle weight, and central nuclei defects associated with LGMD1A in TgT57I mice.

To test the expression of hBAG3 in vivo, ssAAV9.CMV.hBAG3 was injected intramuscularly (IM) into the left gastrocnemius muscles (GAS) of TgT57I transgenic (MYOT) and WT mice at a dose of 2.5×10¹¹ vg. Expression of hBAG3 was detected by Western blot in the injected left muscle lysates 8 weeks post injection, while the BAG3 expression was undetectable in the untreated, control right muscles (FIG. 4, right panel).

Protein aggregation in muscle fibers is the histological hallmark of myofibrillar myopathies and seems to play a key role in their pathogenesis. To confirm the potential for the hBAG3 transgene to reduce protein aggregate accumulation in muscle fibers in vivo, the left GAS of MYOT mice were IM injected with 2.5×10¹¹ vg of ssAAV9.CMV.hBAG3 with the right GAS control muscles serving as the untreated control. Aggregate accumulation was examined by staining ssAAV9.CMV.hBAG3 treated and untreated GAS muscle cryosections with hematoxylin and eosin (H&E) and with a MYOT immunoreactive antibody 12 weeks post AAV delivery.

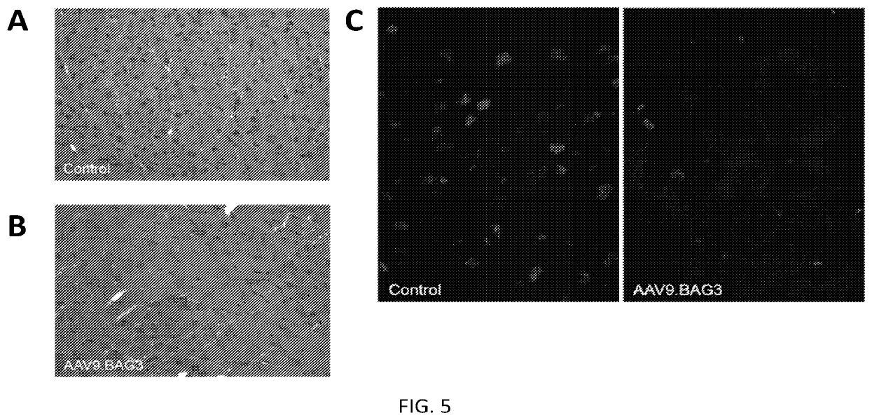

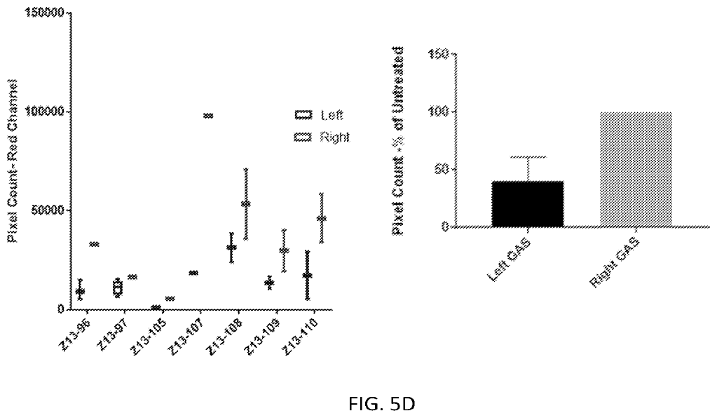

H&E sections from the untreated muscles of the MYOT mice stained a dark purple color indicative of an abundance of intracytoplasmic protein aggregates (FIG. 5A). Compared to the untreated muscles GAS muscles, H&E sections from the treated muscles stained a light purple color indicative of a notable decrease in the amount of intracytoplasmic aggregates (FIG. 5B). These results were confirmed by the immunostaining.

Cryosections of untreated control muscle immunostained with an anti-myotilin antibody displayed high presence of immunoreactive MYOT-seeded protein aggregates while those from treated left GAS muscles showed a significant reduction in MYOT-seeded protein aggregates (FIG. 5C). Quantification of myotilin inclusion density on the fluorescence images from the immunostained sections confirmed that ssAAV9.CMV.hBAG3 treatment significantly reduced the abundance of protein aggregates in TgT57I gastrocnemius muscles, twelve weeks post injection (FIG. 5D). Taken together, these data demonstrate that hBAG3 gene therapy significantly reduced the abundance of intracytoplasmic protein aggregates in the treated left GAS muscles compared to the untreated muscle right muscles.

### Example 6

### Toxicity Assessment and Dose-Escalation Study of AAVrh74.tMCK.hBAG3

A switch from the ssAAV9.CMV.hBAG3 vector to the AAVrh74.tMCK.hBAG3 vector was made to confer long-term sustained high expression of hBAG3 in muscles affected by the disease, while exhibiting limited activity in other tissues. While the strong constitutive CMV promoter in the ssAAV9.CMV.hBAG3 vector is known to drive high levels of gene expression, it is also a promiscuous promoter that is not necessarily ideal for use in restricted muscular transduction. In contrast, the tMCK promoter has high muscle specificity providing high in vivo expression level in skeletal muscle and the heart with both intramuscular and systemic delivery. The tMCK promoter is also compact in size, making it attractive for AAV vectors, as they have a small packaging capacity of ~4.7 Kb. The muscular specificity of the tMCK promoter may be further enhanced by pairing it with the AAVrh74 serotype given its muscle (skeletal and cardiac) tissue tropism making the pairing ideal for muscle gene therapy. Moreover AAVrh.74, isolated from rhesus macaque, has good transduction properties and lower pre-existing human population immunity against it, compared to AAV2 (Mendell et al. JAMA Neurol. 2020; 77 (9): 1122-1131.).

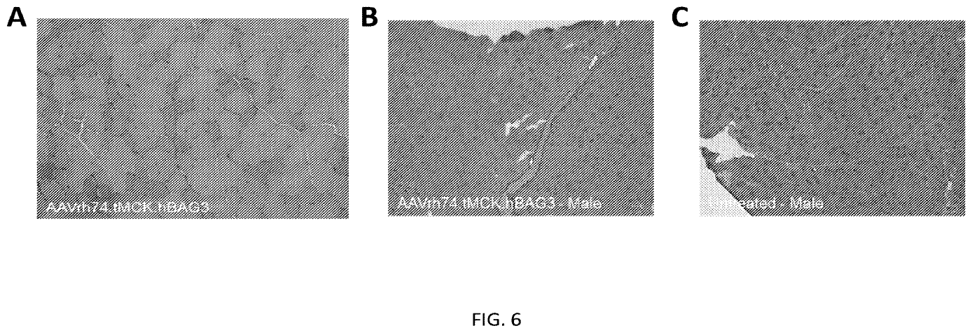

Critical to the success of gene therapy is the demonstration of efficacy at doses of vector that are safe and can be easily manufactured. The toxicity of ssrAAVrh74.tMCK.hBAG3 vector was first assessed in C57BL/6 WT mice (n=4) following IM injection into the right GAS muscles at three different doses: 1×10¹¹, 4×10¹¹, or 8×10¹¹ vg/kg. ssrAAVrh74.tMCK.hBAG3 injection at 1×10¹¹ vg in WT mice did not produce any inflammation or necrosis in the muscle, 8 weeks post injection. With the 4×10¹¹ vg dose, muscle fiber necrosis and minute inflammation in few areas of the muscle were observed in 1 out of 3 mice. However, the 8×10¹¹ vg dose led to widespread inflammation and marked increase of internal nuclei in all mice, and ongoing muscle fiber necrosis in 2 out of 3 mice. These histological observations suggested a dose dependent toxicity following IM injection. Systemic administration of the vector at 3×10¹² vg and 6×10¹² vg in WT did not produce any inflammation or necrosis in the muscle 8 weeks post injection. An increase of fibers with internal nuclei in some areas of GAS, Quad and TA muscles was however noted with the 6×10¹² vg dose, an observation that is compatible with regeneration following previous necrosis (FIG. 6A). Ongoing necrosis or inflammation was not observed.

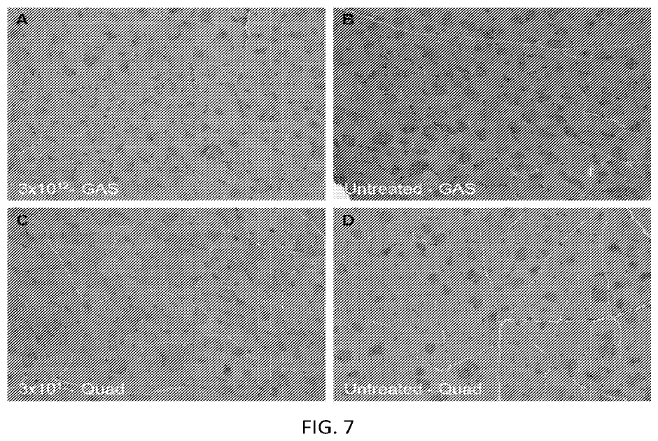

Based on these results in the WT mice, a starting dose of 3×10¹² vg was injected into the tail veins 12-16 weeks old TgT57I transgenic mice (n=3, Table 2) for the systemic delivery of ssrAAVrh74.tMCK.hBAG3. There was marked variability in the extent of aggregate formation among the female mice (presumably due to non-random X-chromosome inactivation) therefore, only males were used in the subsequent experiments assessing the efficacy of BAG3 gene therapy in this mouse model. Eight weeks post systemic delivery of ssrAAVrh74.tMCK.hBAG3 (3×10¹² vg), an overall decrease was observed in the abundance, size, and staining intensity of aggregates in GAS and Quad muscles from a male MYOT mouse compared to control muscles from untreated age-matched male MYOT (Figures B, 6C, and FIG. 7). Aggregates were more basophilic in untreated muscles.

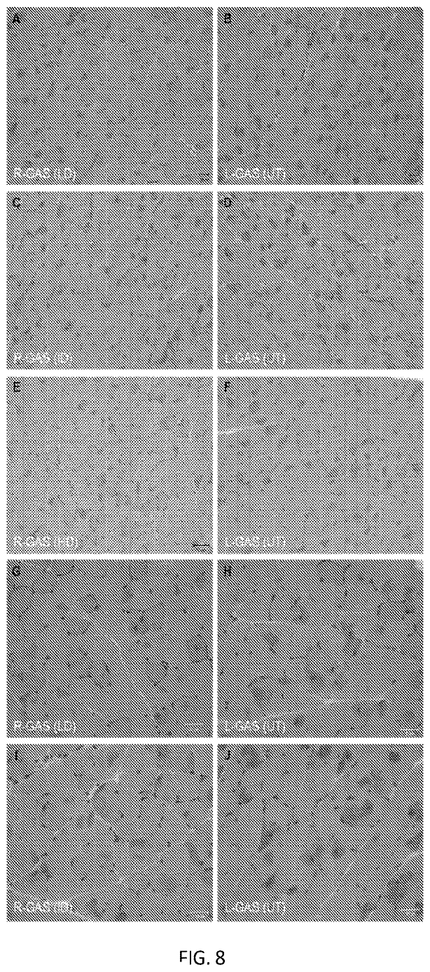

Intramuscular dose titration experiments were performed with a male-only cohort of MYOT mice at three doses of the ssrAAVrh74.tMCK.hBAG3 vector at 3×10¹⁰ vg, 1×10¹¹ vg and 2×10¹¹ vg representing a low dose (LD), intermediate dose (ID), and high dose (HD), respectively. In H&E sections from treated GAS muscles, an apparent decrease in the size and the number of eosinophilic aggregates were noted compared to the uninjected contralateral GAS muscle for all 3 doses (FIG. 8).

TABLE 5
Cohorts of the TgT57I mouse model

| | | | | | Age at | Treatment |
|---|---|---|---|---|---|---|
| Experiment | Group | Delivery | Dose (vg) | n number (F:M) | injection (wks) | duration |
| WT control | | IM - GAS | 1 × 10¹¹ (LD) | 4 (WT mice) | 16-20 | 8 |
| Dose Titration | DT-1 | IM - GAS | 4 × 10¹¹ (ID) | 3 (3F) | 16-20 | 8 |
| | | | 8 × 10¹¹ (HD) | 3 (3F) | 16-20 | 8 |
| | DT-2 | Sys | 3 × 10¹² (LD) | 3 (2F:1M) | 12-16 | 8 |
| | | | 6 × 10¹² (HD) | 1 (1F, WT) | 12-16 | 8 |
| | | | RL | 6 (3F:3M) | 12-16 | 8 |
| | DT-3 | IM - GAS | 3 × 10¹⁰ (LD) | 3 (3M) | 4-5 | 8 |
| | | | 1 × 10¹¹ (ID) | 3 (3M) | 4-5 | 8 |
| | | | 2 × 10¹¹ (HD) | 2 (2M) | 4-5 | 8 |
| Efficacy | E-1 | Sys | 3 × 10¹² | 10 (10M) | 4-5 | |
| | | | RL | 8 (8M)* | 4-5 | |

DT: dose titration, IM: intramuscular, Sys: systemic, GAS: gastrocnemius, LD: low dose, ID: intermediate dose, HD: high dose, RL: Ringer's Lactate, F: female, M: male.

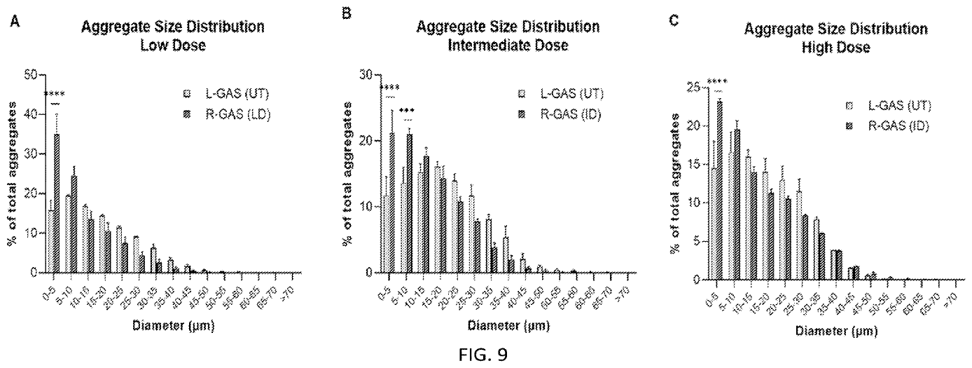

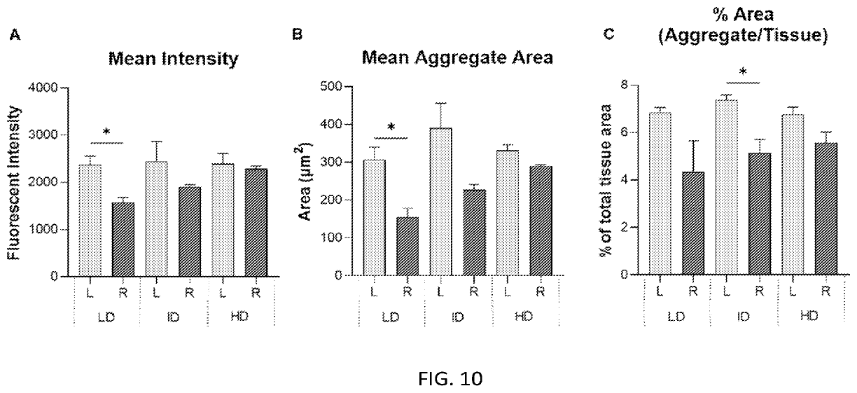

Myotilin aggregate quantification: Quantitative histology was carried out on sections of treated and untreated GAS muscles harvested 8 weeks post vector injection to quantify myotilin positive aggregates and to determine an efficacious vector dose. The sections were immunostained to delineate myotilin positive aggregates. With treatment, a significant shift towards smaller size aggregates was observed in all tested doses (FIG. 9). Mean aggregate fluorescent intensity, mean aggregate area (μm²) and % of area occupied by the aggregates also decreased with BAG3 treatment. Decrease in mean aggregate intensity and mean aggregate area was significant in the low dose (LD) cohort while the % area occupied by the aggregates was significantly lower in the intermediate dose (ID) cohort (Table 6 and FIG. 10).

TABLE 6
Aggregate analysis on cohort DT-3 performed on injected right GAS
muscles (R) and uninjected left GAS muscles (L) for each mouse.

| | | Total | Total | | | Mean | |
|---|---|---|---|---|---|---|---|
| | | Aggregate | Tissue | Mean | Count Of | Aggregate | % |
| Dose | Tissue | Area (μm²) | Area (μm²) | Intensity | Objects | Area (μm²) | Area |
| Low | LD-L | 793266 | 11623563 | 2373 | 2599 | 307 | 6.85 |
| Dose | LD-R | 520593 | 11986933 | 1567 | 3198 | 155 | 4.34 |
| (n = 3) | | | | | | | |
| Int Dose | IntD-L | 855468 | 11600723 | 2443 | 2214 | 391 | 7.38 |
| (n = 3) | IntD-R | 640053 | 12791147 | 1910 | 2817 | 228 | 5.14 |
| High | HD-L | 897994 | 13243050 | 2396 | 2727 | 331 | 6.77 |
| Dose | HD-R | 815759 | 14475150 | 2289 | 2805 | 290 | 5.57 |
| (n = 3) | | | | | | | |

Systemic AAVrh74.tMCK.hBAG3 in the TgT57I Transgenic Mice

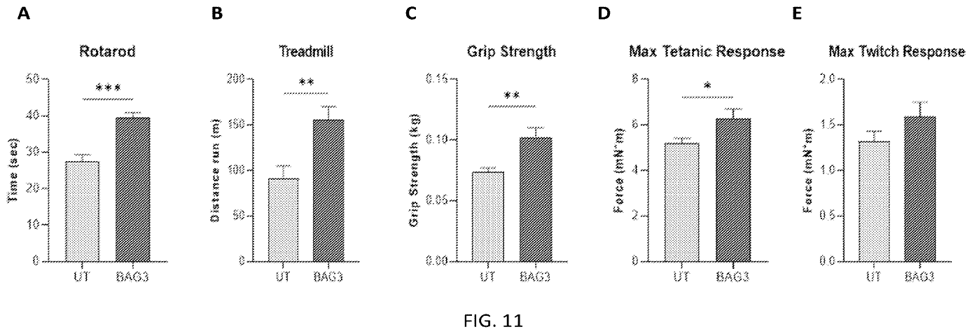

Based on these observations, the 3×10¹² vg dose was chosen as the systemic dose. Ten male mice received 3×10¹² vg dose of AAVrh74.tMCK.hBAG3 at the age of 4-8 weeks. Eight sex and aged-matched TgT57I mice served as the untreated (UT) controls. Functional studies performed on these mice included assays including rotarod, treadmill, grip strength tests, and in vivo muscle contractility assay testing maximum twitch and tetanic responses. At the endpoint (8 months post-injection), the BAG3-treated cohort (n=10) demonstrated increased rotarod duration by 43.2% [BAG3, 39.54±1.33 sec; UT (n=8), 27.6±1.77 sec; p=0.0001] and running distance on treadmill by 70.5% (BAG3, 156.03±13.93 m; UT, 91.53±13.35 m; p=0.0048), compared to UT cohort. Grip-strength values of treated cohort improved by 38.7% (BAG3, 0.102±0.008 kg; UT, 0.074±0.004 kg; p=0.006). The in vivo muscle contractility assay showed that muscle strength was increased significantly for maximum tetanic response (BAG3, 6.26±0.44 mN*m; UT, 5.19±0.23 mN*m; p=0.047), while the increase was not significant for maximum twitch response (FIG. 11A-E).

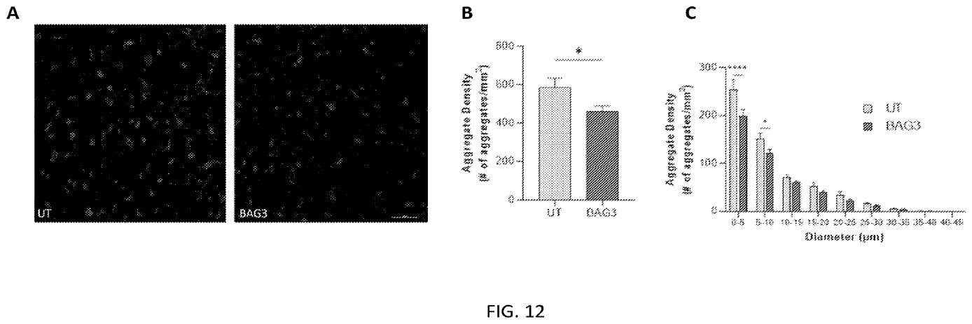

Myotilin aggregate burden in muscle following systemic delivery of AAVrh74.tMCK.hBAG3 was quantified using an immunofluorescence technique, 8 months post-injection. Four randomly selected representative images at 20×-magnification from quadriceps muscles of BAG3-treated (n=10) and UT (n=8) cohorts were analyzed (FIG. 12A). The mean number of myotilin positive aggregates per unit area (aggregate density) had decreased significantly in the treated group, compared to untreated counterparts (BAG3, 466.1±31.3/mm2 vs. UT, 585.6±47.9/mm2; per mouse analysis, p=0.0351; per image analysis, p=0.0002, FIG. 12B). A decrease in mean aggregate fluorescence intensity with treatment, was also noted though it did not reach statistical significance. Aggregate size distribution analysis revealed a reduction of numbers in all aggregate sizes with treatment, demonstrating significance for the aggregate size population up to 10 μm in diameter. (FIG. 12C).

### Example 7

### BAG3 Gene Therapy in SOD1-G93A ALS Mouse Model

The following studies support the hypothesis that acceleration of autophagy via overexpression of BAG3 promotes the degradation of misfolded proteins in neurodegeneration and improve survival and function in SOD1G93A ALS model: 1) Overexpression of SOD1G93A in immortalized motor neurons (NSC34 cells) induced a compensatory increase in the expression of BAG3, HspB8, and Hsp70 and consequently an enhanced removal of SOD1G93A; when autophagy was blocked however, SOD1G93A was not degraded, and 2) SOD1G93A was found to immunoprecipitated with BAG3, HspB8 and Hsc70 indicating the importance of BAG3-mediated autophagy in clearing aggregated, misfolded proteins in familial ALS43. In the spinal cords of SODG93A and SODG85R transgenic mouse models, BAG3 and HspB8 were upregulated compared to control mice, and BAG3 colocalized with the mutant SOD1 in the perinuclear inclusions indicating collaboration of BAG3 with Hsp70 to direct mutant protein to the aggresome for degradation.

Efficacy of AAVrh74.CBA.hBAG3 in SOD1-G93A Mouse

SOD1-G93A mice received systemic administration of ss.AAV9.CBA.hBAG3 (4×10¹² vg) or intramuscular injection of sc.AAV1.tMCK.NT-3 (1×10¹¹ vg), as a monotherapy or combination therapy (Table 7).

TABLE 7
Cohorts for SOD1-G93A model

| | | | | Injection | n |
|---|---|---|---|---|---|
| | | | | Age | num- |
| Cohort | Transgene | Administration | Dose | (days) | ber |
| BAG3-100 | BAG3 | Systemic | 4 × 10¹² vg | 100 | 5 |
| BAG3-70 | BAG3 | Systemic | 4 × 10¹² vg | 70 | 4 |
| BAG3-40 | BAG3 | Systemic | 4 × 10¹² vg | 40 | 3 |
| Combo-40 | BAG3 | Systemic | 4 × 10¹² vg | 40 | 4 |
| | NT-3 | Intramuscular | 11 × 10¹¹ vg | | |
| NT-3-40 | NT-3 | Intramuscular | 11 × 10¹¹ vg | 40 | 3 |

| | | |
|---|---|---|
| RL | Untreated control cohort | 7 |

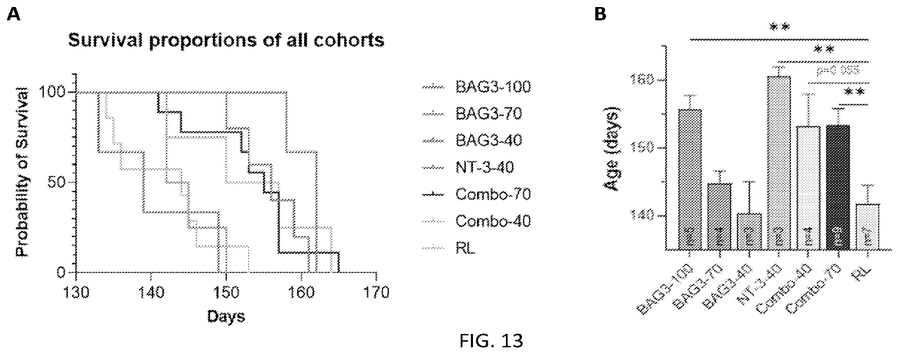

In order to investigate if the time of onset of intraneuronal aggregate formation effects the BAG3 efficacy, systemic BAG3 gene delivery was carried out at P40 [preclinical stage without intraneuronal aggregates (BAG3-40 cohort)], at P70 [early stage of intraneuronal aggregate formation correlating with first notable decline in functional tests (BAG3-70 cohort)] and at P100, a stage with signs of paralysis and more widespread intraneuronal inclusions (BAG3-100 cohort). AAV9.BAG3 systemic delivery at 100 days (BAG3-100), and AAV1.NT-3 IM injection at 40 days (NT-3-40), and the combination therapy at 70 days significantly expanded the life expectancy of SOD1-G93A mice. The mean age of death was 142 days for untreated cohort (FIG. 13) while this number increased to 156 in the BAG3-100 cohort, 161 days in the NT-3-40 cohort, and 153 days in the Combo-70 cohort suggesting a 9.8% (p=0.0073), 13.3% (p=0.0019), and 8.17% (p=0.0099), respectively. The combo group that received the NT-3/BAG3 combination therapy at the age of 40 days also had an almost statistically significant improvement in survival (p=0.055); one out of 4 mice in this cohort survived for 164 days compared to the untreated RL group, corresponding to a 15.6% increase in survival [141.8±2.7 (mean±SE), n=7].

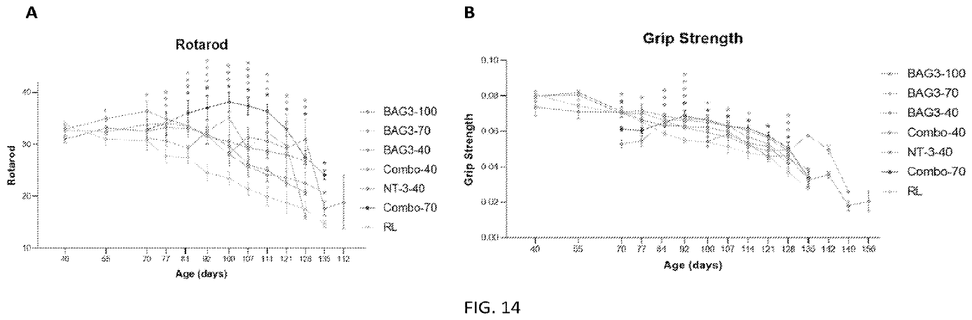

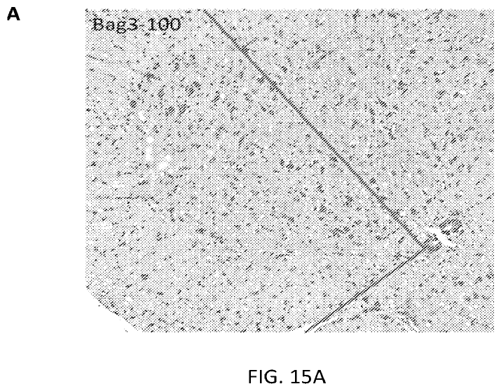

For the functional assays, a steady decline in rotarod, grip strength and electrophysiology test performances was observed in the untreated cohort. In the rotarod test, both BAG3-100, Combo-70, and Combo-40 cohorts showed significant improvements starting one to two weeks post-injection up to 128-135 days old compared to untreated mice (FIG. 14A). NT-3-40, BAG3-70, and BAG3-40 cohorts also performed significantly better for five (77-107 days), four (92-114 days), and three (77-92 days) consecutive weeks, respectively. Overall, performances of NT-3-40 and BAG3-40 cohorts were equally better than the RL cohort. The Combo-40 cohort surpassed the BAG3-40 and NT-3-40 cohorts alone and Combo-70 cohort exceeded the BAG3-70 cohort performing significantly better than the RL cohort throughout the study strongly suggesting that the NT-3/BAG3 combination therapy is more beneficial than either monotherapy. Although the survival time of the Combo-70 cohort did not change compared to Combo-40, the rotarod performance of Combo-70 was significantly better than combo-40 for four consecutive weeks (100-121 days), indicating the duration of the combination therapy might affect the functional improvement. For the BAG3 only cohorts, an overall trend toward better performance in BAG3-70 and BAG3-100 compared to BAG3-40 was observed but it was not statistically significant. Although the duration of BAG3 treatment was critical for the survival time and early treatment did not increase survival, improvements in functional performance was however observed regardless of the injection time of BAG3.

A similar improvement was detected in the grip strength test, predominantly in the Combo-70 and Combo-40 cohorts. These mice had significantly better grip strength for six weeks starting at the 4th week post-injection (FIG. 14B). Although the BAG3-70 cohort had lower grip strength at 70 and 77 days, a remarkable improvement was noted on the 3rd and 4th weeks post-injection. Overall, performances of NT-3-40, BAG3-100, and BAG3-40 cohorts were better than the RL cohort, being significant at 84-92 days, at 128 days, and at 92 days, respectively. Compound muscle action potentials (CMAPs) showed rapid decline in all cohorts at time points tested (every 14-16 days) without statistical difference between groups.

### Quantitative Histopathology:

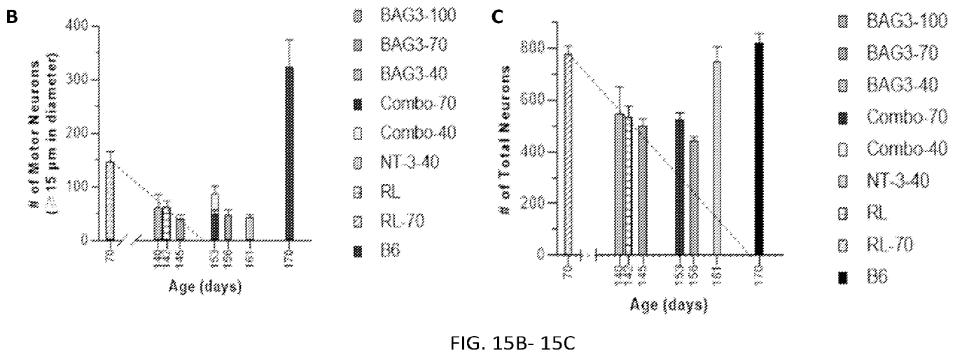

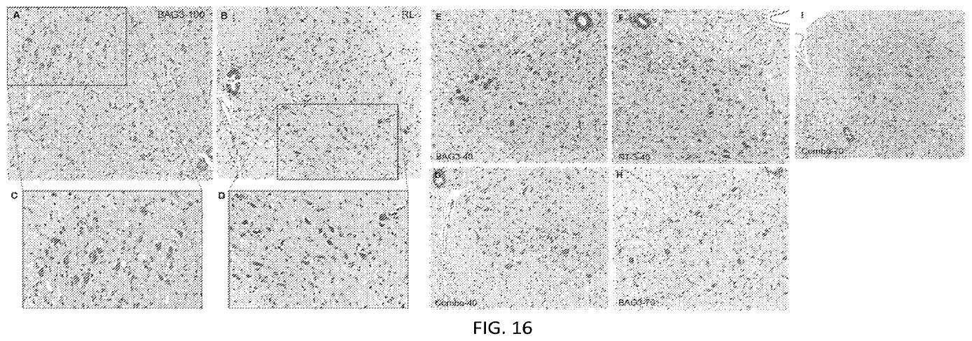

In each treatment cohort, the anterior horn cells from lumbar segments were counted and short axis diameters were measured on cresyl violet-stained paraffin-embedded spinal cord sections at end point. Both large motor neurons as well as small interneurons were included according to parameters detailed in the methods section (FIG. 15).

The quantitative histopathology assessing the number of neurons in the anterior horn area of lumbar segments from each cohort revealed that large neurons (≥15 μm in diameter/motor neurons) were better preserved in Combo-40, Combo 70, BAG3-100 and NT-3-40 cohorts. A total of 147.3 (±18.6) motor neurons in average were counted at age 70 days which decreased to 63.0 (±10.5) at 142 days for untreated mice. The number of motor neurons counted for Combo-40, Combo 70, BAG3-100, and NT-3-40 cohorts were 87.0 (±14.8), 50.2 (±6.5), 48.0 (±9.6), and 44.3 (±3.7), respectively (FIG. 14A). These numbers showed that there was no further motor neuron loss in these cohorts compared to UT, which died 2 weeks earlier, suggesting that the treatment decreased the natural rate of decline in a number of large neurons (FIGS. 15B & 16).

The number of total neurons counted in the anterior horn area of lumbar segments revealed a similar decline in the untreated cohort with time. SOD1.G93A mice had 779.3 (±30.3) total neurons in average at age 70 days while this number decreased to 534.4 (±43.0) at 142 days. The number of total neurons counted for Combo-40, Combo 70, BAG3-100, NT-3-40, and WT cohorts were 526.0 (±25.1), 525.8 (±26.8), 445.4 (±13.4), 750.0 (±56.7), and 821.7 (±36.1) respectively (FIG. 14B). This data suggested that the conservation observed for the large neurons was also valid for the total neuron count in Combo-40, Combo 70, BAG3-100 and NT-3-40 cohorts, being prominent in the NT-3-40 cohort which has a number of neuron-count close to WT and RL-70 data.

### Example 8

### BAG3 Gene Therapy in VCP-A232E Mouse Model

### VCP-A232E Mouse Model of IBMPFD

The VCP-A232E mouse is an inclusion body myositis (IBM) associated with Paget disease of bone and frontotemporal dementia (IBMPFD) model, which was generated and characterized previously by Custer et al. (Hum. Mol. Genet. 19:1741-1755, 2010). The model carries human VCP transgene with A232E mutation which is associated with a severe clinical phenotype characterized by earlier onset and more aggressive myopathy compared to the cases with most commonly observed R155H mutation. The VCP-A232E mouse model, which is a more severe phenotype than VCP-R155H mouse model, accurately recapitulates the complete IBMPFD phenotype as pathology is observed in muscle, brain, and bone. Initial symptoms of muscle weakness are reported around 3-6 months of age, observed as hindlimb clasping.

Histological analyses of quadriceps and gastrocnemius muscles collected from nine months old VCP-A232E mice revealed myopathic changes (including irregular fiber size, centralized nuclei, and inflammatory infiltrates) and rimmed vacuoles. TDP-43 accumulation was also observed in the muscles of this model at nine months. Mice start to show decline in their weights compared to WT mice at around nine months of age.

### Experimental Design

In this model, the transgene is integrated to the X chromosome. Therefore, male mice were used throughout the study to avoid variabilities that can be observed in the females due to X-inactivation. 3×10¹² vg dose of AAVrh74.tMCK.hBAG3 was injected systemically to seven VCP-A232E male mice at three months of age (BAG3 cohort) and eleven mice were used as untreated controls (UT cohort). Mice were euthanized nine months post-gene delivery when they are 12 months old. Mice were tested for rotarod and grip strength at baseline (three months old) and at endpoint (12 months old). Treadmill performance test was performed at endpoint. Gastrocnemius, quadriceps, and tibialis anterior muscles were collected from mice at the endpoint.

### Results

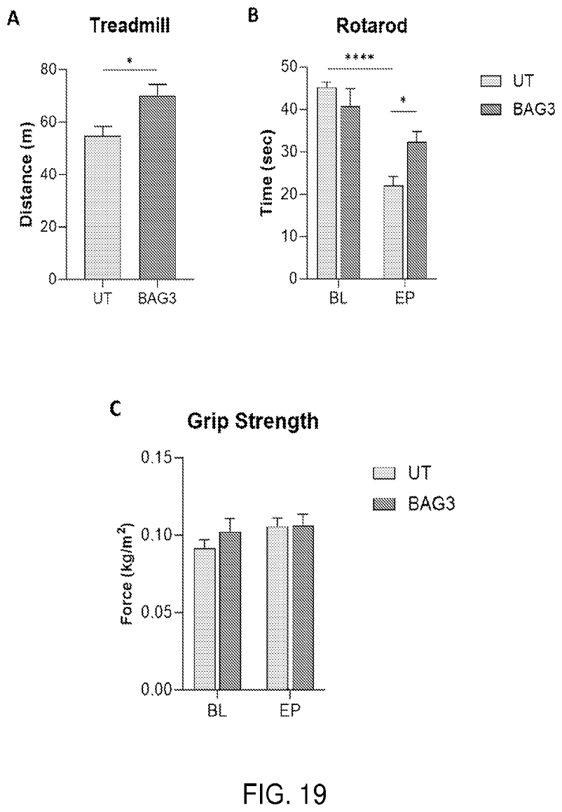

Treadmill performance test showed that the BAG3-treated cohort had longer running distance on treadmill by 27.8% (BAG3, 70.00±4.36 m, n=7; UT, 54.78±3.60 m, n=11; p=0.017), compared to UT cohort at the endpoint (FIG. 19A). Similarly, rotarod test performed at the endpoint revealed that the BAG3 cohort demonstrated increased rotarod duration by 46.2% (BAG3, 32.33±2.47 sec, n=7; UT, 22.12±2.09 sec, n=11; p=0.0197) while no significant decrease was observed between two cohorts at baseline (FIG. 19B). UT cohort showed a decline in rotarod when baseline and endpoint data were compared, compatible with disease progression (UT; Baseline, 45.27±1.17 sec, n=5; Endpoint, 22.12±2.09 sec, n=11; p<0.0001), while BAG3 gene therapy preserved rotarod function (BAG3, Baseline, 40.71±4.18 sec, n=7; Endpoint, 32.33±2.47 sec, n=7; p=0.095). Treatment did not alter the grip strength.

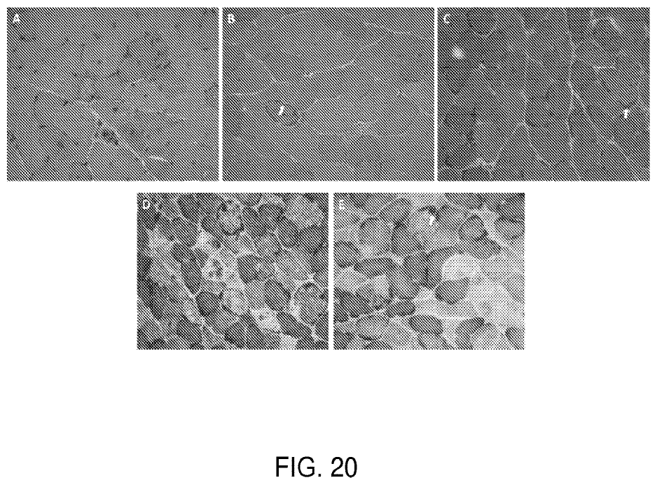

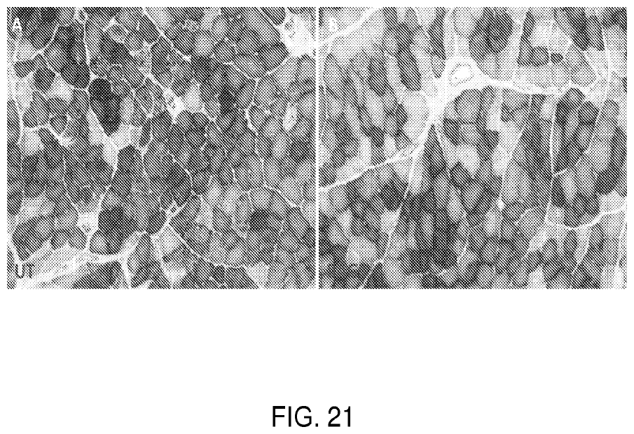

H&E, Gomori Trichrome, COX, and SDH staining were performed on muscle tissues from VCP-A232E mice showing muscle fibers with centralized nuclei indicating degeneration/regeneration process, and mitochondrial abnormalities (ragged red, brown, blue fibers in trichrome, COX, and SDH staining, respectively) shown as subsarcolemmal accumulation of mitochondrial staining and COX negative fibers (FIG. 20). In addition, BAG3 gene therapy improved mitochondrial abnormalities observed in the SDH-stained sections (FIG. 21).

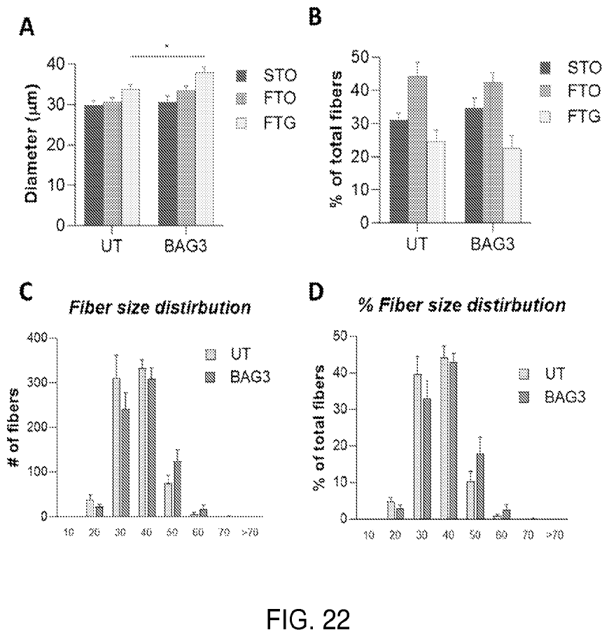

The effects of BAG3 gene therapy in VCP-A232E mice on muscle fiber size and fiber type composition at endpoint were quantified. An increase in the fiber size was observed in all fiber types, and the increase was significant in fast-twitch glycolytic (FTG) fibers in gastrocnemius muscle (BAG3, 38.04±1.23, n=6; UT, 33.81±1.20 μm, p=0.049) (FIG. 22A). Percent distribution of different fiber types didn't show a significant change following BAG3 treatment (FIG. 22B). The treatment effect was reflected in muscle fiber size distribution histograms with a shift to right, larger diameter subgroups (FIG. 22C, D).

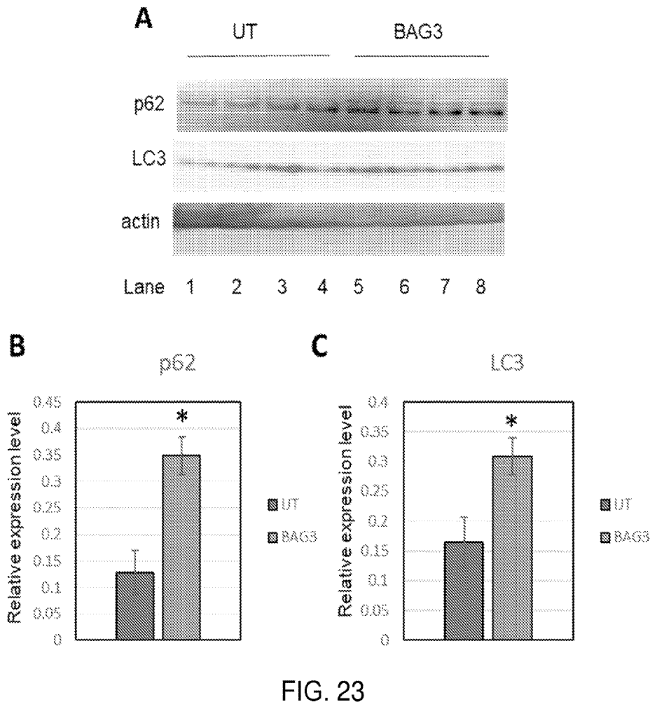

Since BAG3 is one of the key factors of macro-autophagy pathway, the proteins levels of two important autophagy markers: p62 and LC3 proteins were analyzed. A significant increase in both proteins was observed in the treated group compared to untreated counterparts (FIG. 23).

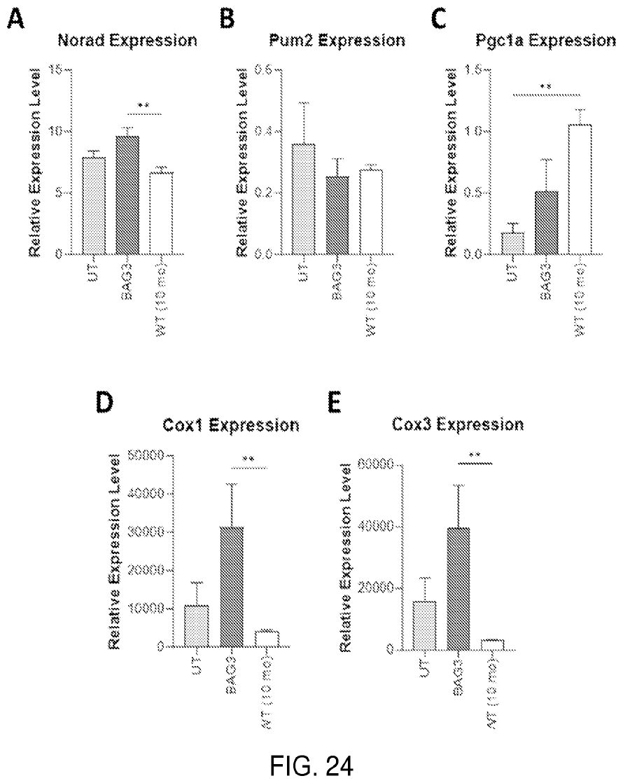

In previous preliminary studies, it was shown that a NORAD (Noncoding RNA activated by DNA damage), which is a long noncoding RNA, might be involved in the evolution of myopathic diseases with a hypothesized protective role against disease progression. To understand how BAG3 gene therapy might affect the expression of NORAD, qRT-PCR was performed on the gastrocnemius muscle of treated and untreated VCP-A232E mice. A trend towards higher levels of Norad was observed in the treated cohort compared to untreated mice; however, the increase observed in this cohort became significant compared to age-matched WT mice (FIG. 24A) indicating that BAG3 treatment might be beneficial in increasing the NORAD levels.

NORAD is the preferred target of RNA binding protein PUM2 in mouse tissues and, upon loss of NORAD, PUM2 hyperactively represses key genes required for mitosis and mitochondrial function. Therefore, Pum2 levels were analyzed and it was observed that Pum2 expression levels were decreased down to WT levels in treated mice (FIG. 24B). PGC1a is a mitochondrial biogenesis marker and COX1 and COX3 are important mtDNA-encoded subunits of COX of respiratory complex IV. A clear trend of increase observed in the Pgc1α levels and the presence of significant increases in the expression of Cox1 and Cox3 subunits compared to untreated cohort suggest a positive effect of BAG3 gene therapy in mitochondrial biogenesis and function (FIG. 24C-E).

These results show that systemic delivery of AAVrh74.tMCK.hBAG3 (3×10¹² vg) VCP-A232E male mice resulted in improvements in functional and histopathological outcome compared to untreated counterparts.

### REFERENCES

1. Harper S Q, Staber P D, He X, et al. RNA interference improves motor and neuropathological abnormalities in a Huntington's disease mouse model. Proc Natl Acad Sci USA 2005; 102:5820-5825.
2. Yamamoto A, Lucas J J, Hen R. Reversal of neuropathology and motor dysfunction in a conditional model of Huntington's disease. Cell 2000; 101:57-66.
3. Zu T, Duvick L A, Kaytor M D, et al. Recovery from polyglutamine-induced neurodegeneration in conditional SCA1 transgenic mice. J Neurosci 2004; 24:8853-8861.
4. Gamerdinger M, Kaya A M, Wolfrum U, Clement A M, Behl C. BAG3 mediates chaperone-based aggresome-targeting and selective autophagy of misfolded proteins. EMBO reports 2011; 12:149-156.
5. Lei Z, Brizzee C, Johnson G V. BAG3 facilitates the clearance of endogenous tau in primary neurons. Neurobiol Aging 2015; 36:241-248.
6. Behl C. Breaking BAG: The Co-Chaperone BAG3 in Health and Disease. Trends Pharmacol Sci 2016; 37:672-688.
7. Meriin A B, Narayanan A, Meng L, et al. Hsp70-Bag3 complex is a hub for proteotoxicity-induced signaling that controls protein aggregation. Proc Natl Acad Sci USA 2018; 115: E7043-E7052.
8. Hishiya A, Salman M N, Carra S, Kampinga H H, Takayama S. BAG3 directly interacts with mutated alphaB-crystallin to suppress its aggregation and toxicity. PloS one 2011; 6: e16828.
9. Fuchs M, Poirier D J, Seguin S J, et al. Identification of the key structural motifs involved in HspB8/HspB6-Bag3 interaction. The Biochemical journal 2009; 425:245-255.
10. McCollum A K, Casagrande G, Kohn E C. Caught in the middle: the role of Bag3 in disease. The Biochemical journal 2009; 425: e1-3.
11. Demand J, Alberti S, Patterson C, Hohfeld J. Cooperation of a ubiquitin domain protein and an E3 ubiquitin ligase during chaperone/proteasome coupling. Current biology: CB 2001; 11:1569-1577.
12. Alberti S, Esser C, Hohfeld J. BAG-1—a nucleotide exchange factor of Hsc70 with multiple cellular functions. Cell Stress Chaperones 2003; 8:225-231.
13. Carra S, Seguin S J, Lambert H, Landry J. HspB8 chaperone activity toward poly(Q)-containing proteins depends on its association with Bag3, a stimulator of macroautophagy. The Journal of biological chemistry 2008; 283:1437-1444.
14. Gamerdinger M, Hajieva P, Kaya A M, Wolfrum U, Hartl F U, Behl C. Protein quality control during aging involves recruitment of the macroautophagy pathway by BAG3. EMBO J 2009; 28:889-901.
15. Minoia M, Boncoraglio A, Vinet J, et al. BAG3 induces the sequestration of proteasomal clients into cytoplasmic puncta: implications for a proteasome-to-autophagy switch. Autophagy 2014; 10:1603-1621.
16. Sturner E, Behl C. The Role of the Multifunctional BAG3 Protein in Cellular Protein Quality Control and in Disease. Frontiers in molecular neuroscience 2017; 10:177.
17. Homma S, Iwasaki M, Shelton G D, Engvall E, Reed J C, Takayama S. BAG3 deficiency results in fulminant myopathy and early lethality. The American journal of pathology 2006; 169:761-773.
18. Selcen D, Muntoni F, Burton B K, et al. Mutation in BAG3 causes severe dominant childhood muscular dystrophy. Ann Neurol 2009; 65:83-89.
19. Lin H, Koren S A, Cvetojevic G, Girardi P, Johnson G V W. The role of BAG3 in health and disease: A “Magic BAG of Tricks”. J Cell Biochem 2022; 123:4-21.
20. Palmio J, Udd B. Myofibrillar and distal myopathies. Revue neurologique 2016; 172:587-593.
21. Cao Y L, Yang Y P, Mao C J, et al. A role of BAG3 in regulating SNCA/alpha-synuclein clearance via selective macroautophagy. Neurobiol Aging 2017; 60:104-115.
22. Faulkner G, Lanfranchi G, Valle G. Telethonin and other new proteins of the Z-disc of skeletal muscle. IUBMB life 2001; 51:275-282.
23. Salmikangas P, van der Ven P F, Lalowski M, et al. Myotilin, the limb-girdle muscular dystrophy 1A (LGMD1A) protein, cross-links actin filaments and controls sarcomere assembly. Hum Mol Genet 2003; 12:189-203.

24 Schilt J, Richoux J P, Dubois M P. [Demonstration of peptides immunologically related SRIF, neurophysins and angiotensin in the planarian Dugesia lugubris (Platyhelminths, Turbellaria) (author's transl)]. J Physiol (Paris) 1981; 77:977-978.
25. Gilchrist J M, Pericak-Vance M, Silverman L, Roses A D. Clinical and genetic investigation in autosomal dominant limb-girdle muscular dystrophy. Neurology 1988; 38:5-9.
26. Hauser M A, Horrigan S K, Salmikangas P, et al. Myotilin is mutated in limb girdle muscular dystrophy 1A. Hum Mol Genet 2000; 9:2141-2147.
27. Maerkens A, Olive M, Schreiner A, et al. New insights into the protein aggregation pathology in myotilinopathy by combined proteomic and immunolocalization analyses. Acta Neuropathol Commun 2016; 4:8.
28. Garvey S M, Miller S E, Claflin D R, Faulkner J A, Hauser M A. Transgenic mice expressing the myotilin T57I mutation unite the pathology associated with LGMD1A and MFM. Hum Mol Genet 2006; 15:2348-2362.
29. Da Cruz S, Cleveland D W. Understanding the role of TDP-43 and FUS/TLS in ALS and beyond. Current opinion in neurobiology 2011; 21:904-919.
30. DeJesus-Hernandez M, Mackenzie I R, Boeve B F, et al. Expanded GGGGCC hexanucleotide repeat in noncoding region of C9ORF72 causes chromosome 9p-linked FTD and ALS. Neuron 2011; 72:245-256.
31. Beckers J, Tharkeshwar A K, Van Damme P. C9orf72 ALS-FTD: recent evidence for dysregulation of the autophagy-lysosome pathway at multiple levels. Autophagy 2021; 17:3306-3322.
32 Rosen D R, Siddique T, Patterson D, et al. Mutations in Cu/Zn superoxide dismutase gene are associated with familial amyotrophic lateral sclerosis. Nature 1993; 362:59-62.
33. Mulligan V K, Chakrabartty A. Protein misfolding in the late-onset neurodegenerative diseases: common themes and the unique case of amyotrophic lateral sclerosis. Proteins 2013; 81:1285-1303.
34. Balch W E, Morimoto R I, Dillin A, Kelly J W. Adapting proteostasis for disease intervention. Science 2008; 319:916-919.
35. Forsberg K, Jonsson P A, Andersen P M, et al. Novel antibodies reveal inclusions containing non-native SOD1 in sporadic ALS patients. PloS one 2010; 5: e11552.
36. Ohi T, Nabeshima K, Kato S, Yazawa S, Takechi S. Familial amyotrophic lateral sclerosis with His46Arg mutation in Cu/Zn superoxide dismutase presenting characteristic clinical features and Lewy body-like hyaline inclusions. J Neurol Sci 2004; 225:19-25.
37. Gurney M E, Pu H, Chiu A Y, et al. Motor neuron degeneration in mice that express a human Cu, Zn superoxide dismutase mutation. Science 1994; 264:1772-1775.
38. Stoica L, Keeler A M, Xiong L, et al. Restrictive Lung Disease in the Cu/Zn Superoxide-Dismutase 1 G93A Amyotrophic Lateral Sclerosis Mouse Model. Am J Respir Cell Mol Biol 2017; 56:405-408.
39. Stieber A, Gonatas J O, Gonatas N K. Aggregates of mutant protein appear progressively in dendrites, in periaxonal processes of oligodendrocytes, and in neuronal and astrocytic perikarya of mice expressing the SOD1 (G93A) mutation of familial amyotrophic lateral sclerosis. J Neurol Sci 2000; 177:114-123.
40. Leigh P N, Whitwell H, Garofalo O, et al. Ubiquitin-immunoreactive intraneuronal inclusions in amyotrophic lateral sclerosis. Morphology, distribution, and specificity. Brain 1991; 114 (Pt 2): 775-788.
41. Cheroni C, Marino M, Tortarolo M, et al. Functional alterations of the ubiquitin-proteasome system in motor neurons of a mouse model of familial amyotrophic lateral sclerosis. Hum Mol Genet 2009; 18:82-96.
42. Liu X, Yamashita T, Shang J, et al. Molecular switching from ubiquitin-proteasome to autophagy pathways in mice stroke model. J Cereb Blood Flow Metab 2020; 40:214-224.
43. Crippa V, Sau D, Rusmini P, et al. The small heat shock protein B8 (HspB8) promotes autophagic removal of misfolded proteins involved in amyotrophic lateral sclerosis (ALS). Hum Mol Genet 2010; 19:3440-3456.
44. Steinacker P, Hawlik A, Lehnert S, et al. Neuroprotective function of cellular prion protein in a mouse model of amyotrophic lateral sclerosis. The American journal of pathology 2010; 176:1409-1420.
45. Shmara A, Perez-Rosendahl M, Murphy K, Kwon A, Smith C, Kimonis V. A clinicopathologic study of malignancy in VCP-associated multisystem proteinopathy. Orphanet J Rare Dis 2022; 17:272.
46. Johnson J O, Mandrioli J, Benatar M, et al. Exome sequencing reveals VCP mutations as a cause of familial ALS. Neuron 2010; 68:857-864.473.
47. Gonzalez M A, Feely S M, Speziani F, et al. A novel mutation in VCP causes Charcot-Marie-Tooth Type 2 disease. Brain 2014; 137:2897-2902.
48. Benatar M, Wuu J, Fernandez C, et al. Motor neuron involvement in multisystem proteinopathy: implications for ALS. Neurology 2013; 80:1874-1880.
49. Korb M K, Kimonis V E, Mozaffar T. Multisystem proteinopathy: Where myopathy and motor neuron disease converge. Muscle Nerve 2021; 63:442-454.
50. Kimonis V E, Mehta S G, Fulchiero E C, et al. Clinical studies in familial VCP myopathy associated with Paget disease of bone and frontotemporal dementia. Am J Med Genet A 2008; 146A: 745-757.
51. Custer S K, Neumann M, Lu H, Wright A C, Taylor J P. Transgenic mice expressing mutant forms VCP/p97 recapitulate the full spectrum of IBMPFD including degeneration in muscle, brain and bone. Hum Mol Genet 2010; 19:1741-1755.

Sequences

SEQ ID NO: 1 - FIG. 17 ssAAVrh74.tMCK.BAG3

| | |
|---|---|
| 1 | GCGCGCTCGC TCGCTCACTG AGGCCGCCCG GGCAAAGCCC GGGCGTCGGG CGACCTTTGG |
| 61 | TCGCCCGGCC TCAGTGAGCG AGCGAGCGCG CAGAGAGGGA GTGGCCAACT CCATCACTAG |
| 121 | GGGTTCCTTA ATACGACTCA CTATAGGGCC GCACCGGTAA GCTTCCACTA CGGGTCTAGG |
| 181 | CTGCCCATGT AAGGAGGCAA GGCCTGGGGA CACCCGAGAT GCCTGGTTAT AATTAACCCC |
| 241 | AACACCTGCT GCCCCCCCCC CCCCAACACC TGCTGCCTGA GCCTGAGCGG TTACCCCACC |
| 301 | CCGGTGCCTG GGTCTTAGGC TCTGTACACC ATGGAGGAGA AGCTCGCTCT AAAAATAACC |
| 361 | CTGTCCCTGG TGGATCCACT ACGGGTCTAG GCTGCCCATG TAAGGAGGCA AGGCCTGGGG |
| 421 | ACACCCGAGA TGCCTGGTTA TAATTAACCC CAACACCTGC TGCCCCCCCC CCCCCAACAC |
| 481 | CTGCTGCCTG AGCCTGAGCG GTTACCCCAC CCCGGTGCCT GGGTCTTAGG CTCTGTACAC |
| 541 | CATGGAGGAG AAGCTCGCTC TAAAAATAAC CCTGTCCCTG GTGGATCCAC TACGGGTCTA |
| 601 | GGCTGCCCAT GTAAGGAGGC AAGGCCTGGG GACACCCGAG ATGCCTGGTT ATAATTAACC |
| 661 | CCAACACCTG CTGCCCCCCC CCCCCCAACA CCTGCTGCCT GAGCCTGAGC GGTTACCCCA |
| 721 | CCCCGGTGCC TGGGTCTTAG GCTCTGTACA CCATGGAGGA GAAGCTCGCT CTAAAAATAA |
| 781 | CCCTGTCCCT GGTGGATCCT CCCTGGGGAC AGCCCCTCCT GGCTAGTCAC ACCCTGTAGG |
| 841 | CTCCTCTATA TAACCCAGGG GCACAGGGGC TGCCCCCGGG TCACGGTGGA TATCCCTGCA |
| 901 | GGTACCACGC GCTGTAATTG AACTGGGAGT GGACACCTGT GGAGAGAAAG GCAAAGTGGA |
| 961 | TGTCAGTAAG ACCAATAGGT GCCTATCAGA AACGCAAGAG TCTTCTCTGT CTCGACAAGC |
| 1021 | CCAGTTTCTA TTGGTCTCCT TAAACCTGTC TTGTAACCTT GATACTTACG GAGAGCAACT |
| 1081 | GCATAAGGGC TAGCCTCGAG AATTCGCCAC CATGAGCGCC GCCACCCACT CGCCCATGAT |
| 1141 | GCAGGTGGCG TCCGGCAACG GTGACCGCGA CCCTTTGCCC CCCGGATGGG AGATCAAGAT |
| 1201 | CGACCCGCAG ACCGGCTGGC CCTTCTTCGT GGACCACAAC AGCCGCACCA CTACGTGGAA |
| 1261 | CGACCCGCGC GTGCCCTCTG AGGGCCCCAA GGAGACTCCA TCCTCTGCCA ATGGCCCTTC |
| 1321 | CCGGGAGGGC TCTAGGCTGC CGCCTGCTAG GGAAGGCCAC CCTGTGTACC CCCAGCTCCG |
| 1381 | ACCAGGCTAC ATTCCCATTC CTGTGCTCCA TGAAGGCGCT GAGAACCGGC AGGTGCACCC |
| 1441 | TTTCCATGTC TATCCCCAGC CTGGGATGCA GCGATTCCGA ACTGAGGCGG CAGCAGCGGC |
| 1501 | TCCTCAGAGG TCCCAGTCAC CTCTGCGGGG CATGCCAGAA ACCACTCAGC CAGATAAACA |
| 1561 | GTGTGGACAG GTGGCAGCGG CGGCGGCAGC CCAGCCCCCA GCCTCCCACG GACCTGAGCG |
| 1621 | GTCCCAGTCT CCAGCTGCCT CTGACTGCTC ATCCTCATCC TCCTCGGCCA GCCTGCCTTC |
| 1681 | CTCCGGCAGG AGCAGCCTGG GCAGTCACCA GCTCCCGCGG GGGTACATCT CCATTCCGGT |
| 1741 | GATACACGAG CAGAACGTTA CCCGGCCAGC AGCCCAGCCC TCCTTCCACC AAGCCCAGAA |
| 1801 | GACGCACTAC CCAGCGCAGC AGGGGGAGTA CCAGACCCAC CAGCCTGTGT ACCACAAGAT |
| 1861 | CCAGGGGGAT GACTGGGAGC CCCGGCCCCT GCGGGCGGCA TCCCCGTTCA GGTCATCTGT |
| 1921 | CCAGGGTGCA TCGAGCCGGG AGGGCTCACC AGCCAGGAGC AGCACGCCAC TCCACTCCCC |
| 1981 | CTCGCCCATC CGTGTGCACA CCGTGGTCGA CAGGCCTCAG CAGCCCATGA CCCATCGAGA |
| 2041 | AACTGCACCT GTTTCCCAGC CTGAAAACAA ACCAGAAAGT AAGCCAGGCC CAGTTGGACC |
| 2101 | AGAACTCCCT CCTGGACACA TCCCAATTCA AGTGATCCGC AAAGAGGTGG ATTCTAAACC |
| 2161 | TGTTTCCCAG AAGCCCCCAC CTCCCTCTGA GAAGGTAGAG GTGAAAGTTC CCCCTGCTCC |
| 2221 | AGTTCCTTGT CCTCCTCCCA GCCCTGGCCC TTCTGCTGTC CCCTCTTCCC CCAAGAGTGT |
| 2281 | GGCTACAGAA GAGAGGGCAG CCCCCAGCAC TGCCCCTGCA GAAGCTACAC CTCCAAAACC |
| 2341 | AGGAGAAGCC GAGGCTCCCC CAAAACATCC AGGAGTGCTG AAAGTGGAAG CCATCCTGGA |
| 2401 | GAAGGTGCAG GGGCTGGAGC AGGCTGTAGA CAACTTTGAA GGCAAGAAGA CTGACAAAAA |
| 2461 | GTACCTGATG ATCGAAGAGT ATTTGACCAA AGAGCTGCTG GCCCTGGATT CAGTGGACCC |
| 2521 | CGAGGGACGA GCCGATGTGC GTCAGGCCAG GAGAGACGGT GTCAGGAAGG TTCAGACCAT |
| 2581 | CTTGGAAAAA CTTGAACAGA AAGCCATTGA TGTCCCAGGT CAAGTCCAGG TCTATGAACT |
| 2641 | CCAGCCCAGC AACCTTGAAG CAGATCAGCC ACTGCAGGCA ATCATGGAGA TGGGTGCCGT |
| 2701 | GGCAGCAGAC AAGGGCAAGA AAAATGCTGG AAATGCAGAA GATCCCCACA CAGAAACCCA |
| 2761 | GCAGCCAGAA GCCACAGCAG CAGCGACTTC AAACCCCAGC AGCATGACAG ACACCCCTGG |
| 2821 | TAACCCAGCA GCACCGTGAG GCGGCCGCGG GGATCCAGAC ATGATAAGAT ACATTGATGA |
| 2881 | GTTTGGACAA ACCACAACTA GAATGCAGTG AAAAAAATGC TTTATTTGTA AAATTTGTGA |
| 2941 | TGCTATTGCT TTATTTGTAA CCATTATAAG CTGCAATAAA CAAGTTAACA ACAACAATTG |
| 3001 | CATTCATTTT ATGTTTCAGG TTCAGGGGGA GGTGTGGGAG GTTTTTTCGG ATCCTCTAGA |
| 3061 | GTCGACCAGA GCATGGCTAC GTAGATAAGT AGCATGGCGG GTTAATCATT AACTACAAGG |
| 3121 | AACCCCTAGT GATGGAGTTG GCCACTCCCT CTCTGCGCGC TCGCTCGCTC ACTGAGGCCG |
| 3181 | GGCGACCAAA GGTCGCCCGA CGCCCGGGCT TTGCCCGGGC GGCCTCAGTG AGCGAGCGAG |
| 3241 | CGCGCAGCTG CATTAATGAA TCGGCCAACG CGCGGGGAGA GGCGGTTTGC GTATTGGGCG |
| 3301 | CTCTTCCGCT TCCTCGCTCA CTGACTCGCT GCGCTCGGTC GTTCGGCTGC GGCGAGCGGT |
| 3361 | ATCAGCTCAC TCAAAGGCGG TAATACGGIT ATCCACAGAA TCAGGGGATA ACGCAGGAAA |
| 3421 | GAACATGTGA GCAAAAGGCC AGCAAAAGGC CAGGAACCGT AAAAAGGCCG CGTTGCTGGC |
| 3481 | GTTTTTCCAT AGGCTCCGCC CCCCTGACGA GCATCACAAA AATCGACGCT CAAGTCAGAG |
| 3541 | GTGGCGAAAC CCGACAGGAC TATAAAGATA CCAGGCGTTT CCCCCTGGAA GCTCCCTCGT |
| 3601 | GCGCTCTCCT GTTCCGACCC TGCCGCTTAC CGGATACCTG TCCGCCTTTC TCCCTTCGGG |
| 3661 | AAGCGTGGCG CTTTCTCATA GCTCACGCTG TAGGTATCTC AGTTCGGTGT AGGTCGTTCG |
| 3721 | CTCCAAGCTG GGCTGTGTGC ACGAACCCCC CGTTCAGCCC GACCGCTGCG CCTTATCCGG |
| 3781 | TAACTATCGT CTTGAGTCCA ACCCGGTAAG ACACGACTTA TCGCCACTGG CAGCAGCCAC |
| 3841 | TGGTAACAGG ATTAGCAGAG CGAGGTATGT ACGCGGTGCT ACAGAGTTCT TGAAGTGGTG |
| 3901 | GCCTAACTAC GGCTACACTA GAAGGACAGT ATTTGGTATC TGCGCTCTGC TGAAGCCAGT |
| 3961 | TACCTTCGGA AAAAGAGTTG GTAGCTCTTG ATCCGGCAAA CAAACCACCG CTGGTAGCGG |
| 4021 | TGGTTTTTTT GTTTGCAAGC AGCAGATTAC GCGCAGAAAA AAAGGATCTC AAGAAGATCC |
| 4081 | TTTGATCTTT TCTACGGGGT CTGACGCTCA GTGGAACGAA AACTCACGTT AAGGGATTTT |
| 4141 | GGTCATGAGA TTATCAAAAA GGATCTTCAC CTAGATCCTT TTAAATTAAA AATGAAGTTT |
| 4201 | TAAATCAATC TAAAGTATAT ATGAGTAAAA ATATTCCGGA ATTGCCAGCT GGGGCGCCCT |
| 4261 | CTGGTAAGGT TGGGAAGCCC TGCAAAGTAA ACTGGATGGC TTTCTTGCCG CCAAGGATCT |
| 4321 | GATGGCGCAG GGGATCAAGA TCTGATCAAG AGACAGGATG AGGATCGTTT CGCATGATTG |
| 4381 | AACAAGATGG ATTGCACGCA GGTTCTCCGG CCGCTTGGGT GGAGAGGCTA TTCGGCTATG |
| 4441 | ACTGGGCACA ACAGACAATC GGCTGCTCTG ATGCCGCCGT GTTCCGGCTG TCAGCGCAGG |
| 4501 | GGCGCCCGGT TCTTTTTGTC AAGACCGACC TGTCCGGTGC CCTGAATGAA CTGCAGGACG |
| 4561 | AGGCAGCGCG GCTATCGTGG CTGGCCACGA CGGGCGTTCC TTGCGCAGCT GTGCTCGACG |
| 4621 | TTGTCACTGA AGCGGGAAGG GACTGGCTGC TATTGGGCGA AGTGCCGGGG CAGGATCTCC |
| 4681 | TGTCATCCCA CCTTGCTCCT GCCGAGAAAG TATCCATCAT GGCTGATGCA ATGCGGCGGC |
| 4741 | TGCATACGCT TGATCCGGCT ACCTGCCCAT TCGACCACCA AGCGAAACAT CGCATCGAGC |
| 4801 | GAGCACGTAC TCGGATGGAA GCCGGTCTTG TCGATCAGGA TGATCTGGAC GAAGAGCATC |
| 4861 | AGGGGCTCGC GCCAGCCGAA CTGTTCGCCA GGCTCAAGGC GCGCATGCCC GACGGCGAGG |
| 4921 | ATCTCGTCGT GACCCATGGC GATGCCTGCT TGCCGAATAT CATGGTGGAA AATGGCCGCT |
| 4981 | TTTCTGGATT CATCGACTGT GGCCGGCTGG GTGTGGCGGA CCGCTATCAG GACATAGCGT |
| 5041 | TGGCTACCCG TGATATTGCT GAAGAGCTTG GCGGCGAATG GGCTGACCGC TTCCTCGTGC |
| 5101 | TTTACGGTAT CGCCGCTCCC GATTCGCAGC GCATCGCCTT CTATCGCCTT CTTGACGAGT |
| 5161 | TCTTCTGAAC CGGTAATATT ATTGAAGCAT TTATCAGGGT TATTGTCTCA TGAGCGGATA |
| 5221 | CATATTTGAA TGTATTTAGA AAAATAAACA AATAGGGGTT CCGCGCACAT TTCCCCGAAA |
| 5281 | AGTGCCACCT GACGTCTAAG AAACCATTAT TATCATGACA TTAACCTATA AAAATAGGCG |
| 5341 | TATCACGAGG CCCTTTCGTC TCGCGCGTTT CGGTGATGAC GGTGAAAACC TCTGACACAT |
| 5401 | GCAGCTCCCG GAGACGGTCA CAGCTTGTCT GTAAGCGGAT GCCGGGAGCA GACAAGCCCG |
| 5461 | TCAGGGCGCG TCAGCGGGTG TTGGCGGGTG TCGGGGCTGG CTTAACTATG CGGCATCAGA |
| 5521 | GCAGATTGTA CTGAGAGTGC ACCATATGCG GTGTGAAATA CCGCACAGAT GCGTAAGGAG |
| 5581 | AAAATACCGC ATCAGGAACT TCCAACATCC AATAAATCAT ACAGGCAAGG CAAAGAATTA |
| 5641 | GCAAAATTAA GCAATAAAGC CTCAGAGCAT AAAGCTAAAT CGGTTGTACC AAAAACATTA |
| 5701 | TGACCCTGTA ATACTTTTGC GGGAGAAGCC TTTATTTCAA CGCAAGGATA AAAATTTTTA |
| 5761 | GAACCCTCAT ATATTTTAAA TGCAATGCCT GAGTAATGTG TAGGTAAAGA TTCAAACGGG |
| 5821 | TGAGAAAGGC CGGAGACAGT CAAATCACCA TCAATATGAT ATTCAACCGT TCTAGCTGAT |
| 5881 | AAATTCATGC CGGAGAGGGT AGCTATTTTT GAGAGGTCTC TACAAAGGCT ATCAGGTCAT |
| 5941 | TGCCTGAGAG TCTGGAGCAA ACAAGAGAAT CGATGAACGG TAATCGTAAA ACTAGCATGT |
| 6001 | CAATCATATG TACCCCGGTT GATAATCAGA AAAGCCCCAA AAACAGGAAG ATTGTATAAG |
| 6061 | CAAATATTTA AATTGTAAAC GTTAATATTT TGTTAAAATT CGCGTTAAAT TTTTGTTAAA |
| 6121 | TCAGCTCATT TTTTAACCAA TAGGCCGAAA TCGGCAAAAT CCCTTATAAA TCAAAAGAAT |
| 6181 | AGACCGAGAT AGGGTTGAGT GTTGTTCCAG TTTGGAACAA GAGTCCACTA TTAAAGAACG |
| 6241 | TGGACTCCAA CGTCAAAGGG CGAAAAACCG TCTATCAGGG CGATGGCCCA CTACGTGAAC |
| 6301 | CATCACCCTA ATCAAGTTTT TTGGGGTCGA GGTGCCGTAA ATCACTAAAT CGGAACCCTA |
| 6361 | AAGGGAGCCC CCGATTTAGA GCTTGACGGG GAAAGCCGGC GAACGTGGCG AGAAAGGAAG |
| 6421 | GGAAGAAAGC GAAAGGAGCG GGCGCTAGGG CGCTGGCAAG TGTAGCGGTC ACGCTGCGCG |
| 6481 | TAACCACCAC ACCCGCCGCG CTTAATGCGC CGCTACAGGG CGCGTACTAT GGTTGCTTTG |
| 6541 | ACGAGCACGT ATAACGTGCT TTCCTCGTTA GAATCAGAGC GGGAGCTAAA CAGGAGGCCG |
| 6601 | ATTAAAGGGA TTTTAGACAG GAACGGTACG CCAGAATCCT GAGAAGTGTT TTTATAATCA |
| 6661 | GTGAGGCCAC CGAGTAAAAG AGTCTGTCCA TCACGCAAAT TAACCGTTGT CGCAATACTT |
| 6721 | CTTTGATTAG TAATAACATC ACTTGCCTGA GTAGAAGAAC TCAAACTATC GGCCTTGCTG |
| 6781 | GTAATATCCA GAACAATATT ACCGCCAGCC ATTGCAACGG AATCGCCATT CGCCATTCAG |
| 6841 | GCTGCGCAAC TGTTGGGAAG GGCGATCGGT GCGGGCCTCT TCGCTATTAC GCCAGCT |
| // | |

SEQ ID NO: 2 - FIG. 18 ssAAV9.CMV.CBA.BAG3

| | |
|---|---|
| 1 | GCGCGCTCGC TCGCTCACTG AGGCCGCCCG GGCAAAGCCC GGGCGTCGGG CGACCTTTGG |
| 61 | TCGCCCGGCC TCAGTGAGCG AGCGAGCGCG CAGAGAGGGA GTGGCCAACT CCATCACTAG |
| 121 | GGGTTCCTTA ATACGACTCA CTATAGGGCC GCACCGGTAA GCTTTTCACG CGTGGATCTG |
| 181 | AATTCAATTC ACGCGTGGTA CCTCTGGTCG TTACATAACT TACGGTAAAT GGCCCGCCTG |
| 241 | GCTGACCGCC CAACGACCCC CGCCCATTGA CGTCAATAAT GACGTATGTT CCCATAGTAA |
| 301 | CGCCAATAGG GACTTTCCAT TGACGTCAAT GGGTGGAGTA TTTACGGTAA ACTGCCCACT |
| 361 | TGGCAGTACA TCAAGTGTAT CATATGCCAA GTACGCCCCC TATTGACGTC AATGACGGTA |
| 421 | AATGGCCCGC CTGGCATTAT GCCCAGTACA TGACCTTATG GGACTTTCCT ACTTGGCAGT |
| 481 | ACATCTACTC GAGGCCACGT TCTGCTTCAC TCTCCCCATC TCCCCCCCCT CCCCACCCCC |
| 541 | AATTTTGTAT TTATTTATTT TTTAATTATT TTGTGCAGCG ATGGGGGCGG GGGGGGGGGG |
| 601 | GGGGCGCGCG CCAGGCGGGG CGGGGCGGGG CGAGGGGCGG GGCGGGGCGA GGCGGAGAGG |
| 661 | TGCGGCGGCA GCCAATCAGA GCGGCGCGCT CCGAAAGTTT CCTTTTATGG CGAGGCGGCG |
| 721 | GCGGCGGCGG CCCTATAAAA AGCGAAGCGC GCGGCGGGCG GGAGCGGGAT CAGCCACCGC |
| 781 | GGTGGCGGCC TAGAGTCGAC GAGGAACTGA AAAACCAGAA AGTTAACTGG TAAGTTTAGT |
| 841 | CTTTTTGTCT TTTATTICAG GTCCCGGATC CGGTGGTGGT GCAAATCAAA GAACTGCTCC |
| 901 | TCAGTGGATG TTGCCTTTAC TTCTAGGCCT GTACGGAAGT GTTACTTCTG CTCTAAAAGC |
| 961 | TGCGGAATGA ATTCGCCACC ATGAGCGCCG CCACCCACTC GCCCATGATG CAGGTGGCGT |
| 1021 | CCGGCAACGG TGACCGCGAC CCTTTGCCCC CCGGATGGGA GATCAAGATC GACCCGCAGA |
| 1081 | CCGGCTGGCC CTTCTTCGTG GACCACAACA GCCGCACCAC TACGTGGAAC GACCCGCGCG |
| 1141 | TGCCCTCTGA GGGCCCCAAG GAGACTCCAT CCTCTGCCAA TGGCCCTTCC CGGGAGGGCT |
| 1201 | CTAGGCTGCC GCCTGCTAGG GAAGGCCACC CTGTGTACCC CCAGCTCCGA CCAGGCTACA |
| 1261 | TTCCCATTCC TGTGCTCCAT GAAGGCGCTG AGAACCGGCA GGTGCACCCT TTCCATGTCT |
| 1321 | ATCCCCAGCC TGGGATGCAG CGATTCCGAA CTGAGGCGGC AGCAGCGGCT CCTCAGAGGT |
| 1381 | CCCAGTCACC TCTGCGGGGC ATGCCAGAAA CCACTCAGCC AGATAAACAG TGTGGACAGG |
| 1441 | TGGCAGCGGC GGCGGCAGCC CAGCCCCCAG CCTCCCACGG ACCTGAGCGG TCCCAGTCTC |
| 1501 | CAGCTGCCTC TGACTGCTCA TCCTCATCCT CCTCGGCCAG CCTGCCTTCC TCCGGCAGGA |
| 1561 | GCAGCCTGGG CAGTCACCAG CTCCCGCGGG GGTACATCTC CATTCCGGTG ATACACGAGC |
| 1621 | AGAACGTTAC CCGGCCAGCA GCCCAGCCCT CCTTCCACCA AGCCCAGAAG ACGCACTACC |
| 1681 | CAGCGCAGCA GGGGGAGTAC CAGACCCACC AGCCTGTGTA CCACAAGATC CAGGGGGATG |
| 1741 | ACTGGGAGCC CCGGCCCCTG CGGGCGGCAT CCCCGTTCAG GTCATCTGTC CAGGGTGCAT |
| 1801 | CGAGCCGGGA GGGCTCACCA GCCAGGAGCA GCACGCCACT CCACTCCCCC TCGCCCATCC |
| 1861 | GTGTGCACAC CGTGGTCGAC AGGCCTCAGC AGCCCATGAC CCATCGAGAA ACTGCACCTG |
| 1921 | TTTCCCAGCC TGAAAACAAA CCAGAAAGTA AGCCAGGCCC AGTTGGACCA GAACTCCCTC |
| 1981 | CTGGACACAT CCCAATTCAA GTGATCCGCA AAGAGGTGGA TTCTAAACCT GTTTCCCAGA |
| 2041 | AGCCCCCACC TCCCTCTGAG AAGGTAGAGG TGAAAGTTCC CCCTGCTCCA GTTCCTTGTC |
| 2101 | CTCCTCCCAG CCCTGGCCCT TCTGCTGTCC CCTCTTCCCC CAAGAGTGTG GCTACAGAAG |
| 2161 | AGAGGGCAGC CCCCAGCACT GCCCCTGCAG AAGCTACACC TCCAAAACCA GGAGAAGCCG |
| 2221 | AGGCTCCCCC AAAACATCCA GGAGTGCTGA AAGTGGAAGC CATCCTGGAG AAGGTGCAGG |
| 2281 | GGCTGGAGCA GGCTGTAGAC AACTTTGAAG GCAAGAAGAC TGACAAAAAG TACCTGATGA |
| 2341 | TCGAAGAGTA TTTGACCAAA GAGCTGCTGG CCCTGGATTC AGTGGACCCC GAGGGACGAG |
| 2401 | CCGATGTGCG TCAGGCCAGG AGAGACGGTG TCAGGAAGGT TCAGACCATC TTGGAAAAAC |
| 2461 | TTGAACAGAA AGCCATTGAT GTCCCAGGTC AAGTCCAGGT CTATGAACTC CAGCCCAGCA |
| 2521 | ACCTTGAAGC AGATCAGCCA CTGCAGGCAA TCATGGAGAT GGGTGCCGTG GCAGCAGACA |
| 2581 | AGGGCAAGAA AAATGCTGGA AATGCAGAAG ATCCCCACAC AGAAACCCAG CAGCCAGAAG |
| 2641 | CCACAGCAGC AGCGACTTCA AACCCCAGCA GCATGACAGA CACCCCTGGT AACCCAGCAG |
| 2701 | CACCGTGAGG CGGCCGCGGG GATCCAGACA TGATAAGATA CATTGATGAG TTTGGACAAA |
| 2761 | CCACAACTAG AATGCAGTGA AAAAAATGCT TTATTTGTAA AATTTGTGAT GCTATTGCTT |
| 2821 | TATTTGTAAC CATTATAAGC TGCAATAAAC AAGTTAACAA CAACAATTGC ATTCATTTTA |
| 2881 | TGTTTCAGGT TCAGGGGGAG GTGTGGGAGG TTTTTTCGGA TCCTCTAGAG TCGACCAGAG |
| 2941 | CATGGCTACG TAGATAAGTA GCATGGCGGG TTAATCATTA ACTACAAGGA ACCCCTAGTG |
| 3001 | ATGGAGTTGG CCACTCCCTC TCTGCGCGCT CGCTCGCTCA CTGAGGCCGG GCGACCAAAG |
| 3061 | GTCGCCCGAC GCCCGGGCTT TGCCCGGGCG GCCTCAGTGA GCGAGCGAGC GCGCAGCTGC |
| 3121 | ATTAATGAAT CGGCCAACGC GCGGGGAGAG GCGGTTTGCG TATTGGGCGC TCTTCCGCTT |
| 3181 | CCTCGCTCAC TGACTCGCTG CGCTCGGTCG TTCGGCTGCG GCGAGCGGTA TCAGCTCACT |
| 3241 | CAAAGGCGGT AATACGGTTA TCCACAGAAT CAGGGGATAA CGCAGGAAAG AACATGTGAG |
| 3301 | CAAAAGGCCA GCAAAAGGCC AGGAACCGTA AAAAGGCCGC GTTGCTGGCG TTTTTCCATA |
| 3361 | GGCTCCGCCC CCCTGACGAG CATCACAAAA ATCGACGCTC AAGTCAGAGG TGGCGAAACC |
| 3421 | CGACAGGACT ATAAAGATAC CAGGCGTTTC CCCCTGGAAG CTCCCTCGTG CGCTCTCCTG |
| 3481 | TTCCGACCCT GCCGCTTACC GGATACCTGT CCGCCTTTCT CCCTTCGGGA AGCGTGGCGC |
| 3541 | TTTCTCATAG CTCACGCTGT AGGTATCTCA GTTCGGTGTA GGTCGTTCGC TCCAAGCTGG |
| 3601 | GCTGTGTGCA CGAACCCCCC GTTCAGCCCG ACCGCTGCGC CTTATCCGGT AACTATCGTC |
| 3661 | TTGAGTCCAA CCCGGTAAGA CACGACTTAT CGCCACTGGC AGCAGCCACT GGTAACAGGA |
| 3721 | TTAGCAGAGC GAGGTATGTA GGCGGTGCTA CAGAGTTCTT GAAGTGGTGG CCTAACTACG |
| 3781 | GCTACACTAG AAGGACAGTA TTTGGTATCT GCGCTCTGCT GAAGCCAGTT ACCTTCGGAA |
| 3841 | AAAGAGTTGG TAGCTCTTGA TCCGGCAAAC AAACCACCGC TGGTAGCGGT GGTTTTTTTG |
| 3901 | TTTGCAAGCA GCAGATTACG CGCAGAAAAA AAGGATCTCA AGAAGATCCT TTGATCTTTT |
| 3961 | CTACGGGGTC TGACGCTCAG TGGAACGAAA ACTCACGTTA AGGGATTTTG GTCATGAGAT |
| 4021 | TATCAAAAAG GATCTTCACC TAGATCCTTT TAAATTAAAA ATGAAGTTTT AAATCAATCT |
| 4081 | AAAGTATATA TGAGTAAAAA TATTCCGGAA TTGCCAGCTG GGGCGCCCTC TGGTAAGGTT |
| 4141 | GGGAAGCCCT GCAAAGTAAA CTGGATGGCT TTCTTGCCGC CAAGGATCTG ATGGCGCAGG |
| 4201 | GGATCAAGAT CTGATCAAGA GACAGGATGA GGATCGTTTC GCATGATTGA ACAAGATGGA |
| 4261 | TTGCACGCAG GTTCTCCGGC CGCTTGGGTG GAGAGGCTAT TCGGCTATGA CTGGGCACAA |
| 4321 | CAGACAATCG GCTGCTCTGA TGCCGCCGTG TTCCGGCTGT CAGCGCAGGG GCGCCCGGTT |
| 4381 | CTTTTTGTCA AGACCGACCT GTCCGGTGCC CTGAATGAAC TGCAGGACGA GGCAGCGCGG |
| 4441 | CTATCGTGGC TGGCCACGAC GGGCGTTCCT TGCGCAGCTG TGCTCGACGT TGTCACTGAA |
| 4501 | GCGGGAAGGG ACTGGCTGCT ATTGGGCGAA GTGCCGGGGC AGGATCTCCT GTCATCCCAC |
| 4561 | CTTGCTCCTG CCGAGAAAGT ATCCATCATG GCTGATGCAA TGCGGCGGCT GCATACGCTT |
| 4621 | GATCCGGCTA CCTGCCCATT CGACCACCAA GCGAAACATC GCATCGAGCG AGCACGTACT |
| 4681 | CGGATGGAAG CCGGTCTTGT CGATCAGGAT GATCTGGACG AAGAGCATCA GGGGCTCGCG |
| 4741 | CCAGCCGAAC TGTTCGCCAG GCTCAAGGCG CGCATGCCCG ACGGCGAGGA TCTCGTCGTG |
| 4801 | ACCCATGGCG ATGCCTGCTT GCCGAATATC ATGGTGGAAA ATGGCCGCTT TTCTGGATTC |
| 4861 | ATCGACTGTG GCCGGCTGGG TGTGGCGGAC CGCTATCAGG ACATAGCGTT GGCTACCCGT |
| 4921 | GATATTGCTG AAGAGCTTGG CGGCGAATGG GCTGACCGCT TCCTCGTGCT TTACGGTATC |
| 4981 | GCCGCTCCCG ATTCGCAGCG CATCGCCTTC TATCGCCTTC TTGACGAGTT CTTCTGAACC |
| 5041 | GGTAATATTA TTGAAGCATT TATCAGGGTT ATTGTCTCAT GAGCGGATAC ATATTTGAAT |
| 5101 | GTATTTAGAA AAATAAACAA ATAGGGGTTC CGCGCACATT TCCCCGAAAA GTGCCACCTG |
| 5161 | ACGTCTAAGA AACCATTATT ATCATGACAT TAACCTATAA AAATAGGCGT ATCACGAGGC |
| 5221 | CCTTTCGTCT CGCGCGTTTC GGTGATGACG GTGAAAACCT CTGACACATG CAGCTCCCGG |
| 5281 | AGACGGTCAC AGCTTGTCTG TAAGCGGATG CCGGGAGCAG ACAAGCCCGT CAGGGCGCGT |
| 5341 | CAGCGGGTGT TGGCGGGTGT CGGGGCTGGC TTAACTATGC GGCATCAGAG CAGATTGTAC |
| 5401 | TGAGAGTGCA CCATATGCGG TGTGAAATAC CGCACAGATG CGTAAGGAGA AAATACCGCA |
| 5461 | TCAGGAACTT CCAACATCCA ATAAATCATA CAGGCAAGGC AAAGAATTAG CAAAATTAAG |
| 5521 | CAATAAAGCC TCAGAGCATA AAGCTAAATC GGTTGTACCA AAAACATTAT GACCCTGTAA |
| 5581 | TACTTTTGCG GGAGAAGCCT TTATTTCAAC GCAAGGATAA AAATTTTTAG AACCCTCATA |
| 5641 | TATTTTAAAT GCAATGCCTG AGTAATGTGT AGGTAAAGAT TCAAACGGGT GAGAAAGGCC |
| 5701 | GGAGACAGTC AAATCACCAT CAATATGATA TTCAACCGTT CTAGCTGATA AATTCATGCC |
| 5761 | GGAGAGGGTA GCTATTTTTG AGAGGTCTCT ACAAAGGCTA TCAGGTCATT GCCTGAGAGT |
| 5821 | CTGGAGCAAA CAAGAGAATC GATGAACGGT AATCGTAAAA CTAGCATGTC AATCATATGT |
| 5881 | ACCCCGGTTG ATAATCAGAA AAGCCCCAAA AACAGGAAGA TTGTATAAGC AAATATTTAA |
| 5941 | ATTGTAAACG TTAATATTTT GTTAAAATTC GCGTTAAATT TTTGTTAAAT CAGCTCATTT |
| 6001 | TTTAACCAAT AGGCCGAAAT CGGCAAAATC CCTTATAAAT CAAAAGAATA GACCGAGATA |
| 6061 | GGGTTGAGTG TTGTTCCAGT TTGGAACAAG AGTCCACTAT TAAAGAACGT GGACTCCAAC |
| 6121 | GTCAAAGGGC GAAAAACCGT CTATCAGGGC GATGGCCCAC TACGTGAACC ATCACCCTAA |
| 6181 | TCAAGTTTTT TGGGGTCGAG GTGCCGTAAA TCACTAAATC GGAACCCTAA AGGGAGCCCC |
| 6241 | CGATTTAGAG CTTGACGGGG AAAGCCGGCG AACGTGGCGA GAAAGGAAGG GAAGAAAGCG |
| 6301 | AAAGGAGCGG GCGCTAGGGC GCTGGCAAGT GTAGCGGTCA CGCTGCGCGT AACCACCACA |
| 6361 | CCCGCCGCGC TTAATGCGCC GCTACAGGGC GCGTACTATG GTTGCTTTGA CGAGCACGTA |
| 6421 | TAACGTGCTT TCCTCGTTAG AATCAGAGCG GGAGCTAAAC AGGAGGCCGA TTAAAGGGAT |
| 6481 | TTTAGACAGG AACGGTACGC CAGAATCCTG AGAAGTGTTT TTATAATCAG TGAGGCCACC |
| 6541 | GAGTAAAAGA GTCTGTCCAT CACGCAAATT AACCGTTGTC GCAATACTTC TTTGATTAGT |
| 6601 | AATAACATCA CTTGCCTGAG TAGAAGAACT CAAACTATCG GCCTTGCTGG TAATATCCAG |
| 6661 | AACAATATTA CCGCCAGCCA TTGCAACGGA ATCGCCATTC GCCATTCAGG CTGCGCAACT |
| 6721 | GTTGGGAAGG GCGATCGGTG CGGGCCTCTT CGCTATTACG CCAGCT |

SEQ ID NO: 3 nucleotides 1112 . . . 2839 of SEQ ID NO: 1 (1728 bp)
SEQ ID NO: 4 nucleotides 981 . . . 2708 of SEQ ID NO: 2 (1728 bp)
BAG3 protein SEQ ID NO: 5

| | |
|---|---|
| 1 | msaathspmm qvasgngdrd plppgweiki dpqtgwpffv dhnsrtttwn dprvpsegpk |
| 61 | etpssangps regsrlppar eghpvypqlr pgyipipvlh egaenrqvhp fhvypqpgmq |
| 121 | rfrteaaaaa pqrsqsplrg mpettqpdkq cgqvaaaaaa qppashgper sqspaasdcs |
| 181 | sssssaslps sgrsslgshq lprgyisipv iheqnvtrpa aqpsfhqaqk thypaqqgey |
| 241 | qthqpvyhki qgddweprpl raaspfrssv qgassregsp arsstplhsp spirvhtvvd |
| 301 | rpqqpmthre tapvsqpenk peskpgpvgp elppghipiq virkevdskp vsqkppppse |
| 361 | kvevkvppap vpcpppspgp savpsspksv ateeraapst apaeatppkp geaeappkhp |
| 421 | gvlkveaile kvqgleqavd nfegkktdkk ylmieeyltk ellaldsvdp egradvrqar |
| 481 | rdgvrkvqti lekleqkaid vpgqvqvyel qpsnleadqp lqaimemgav aadkgkknag |
| 541 | naedphtetq qpeataaats npssmtdtpg npaap |

SEQ ID NO: 6 rAAV genome nucleotides 1-3245 of SEQ ID NO: 1
SEQ ID No: 7 rAAV genome nucleotides 1-3114 of SEQ ID NO: 2
SEQ ID NO: 8 BAG3 DNA (genbank: NM_004281.4)

| | |
|---|---|
| 1 | gcatccaacc ccgggccgcg gccaacttct ctggactgga ccagaagttt ctagccggcc |
| 61 | agttgctacc tccctttatc tcctccttcc cctctggcag cgaggaggct atttccagac |
| 121 | acttccaccc ctctctggcc acgtcacccc cgcctttaat tcataaaggt gcccggcgcc |
| 181 | ggcttcccgg acacgtcggc ggcggagagg ggcccacggc ggcggcccgg ccagagactc |
| 241 | ggcgcccgga gccagcgccc cgcacccgcg ccccagcggg cagaccccaa cccagcatga |
| 301 | gcgccgccac ccactcgccc atgatgcagg tggcgtccgg caacggtgac cgcgaccctt |
| 361 | tgccccccgg atgggagatc aagatcgacc cgcagaccgg ctggcccttc ttcgtggacc |
| 421 | acaacagccg caccactacg tggaacgacc cgcgcgtgcc ctctgagggc cccaaggaga |
| 481 | ctccatcctc tgccaatggc ccttcccggg agggctctag gctgccgcct gctagggaag |
| 541 | gccaccctgt gtacccccag ctccgaccag gctacattcc cattcctgtg ctccatgaag |
| 601 | gcgctgagaa ccggcaggtg caccctttcc atgtctatcc ccagcctggg atgcagcgat |
| 661 | tccgaactga ggcggcagca gcggctcctc agaggtccca gtcacctctg cggggcatgc |
| 721 | cagaaaccac tcagccagat aaacagtgtg gacaggtggc agcggcggcg gcagcccagc |
| 781 | ccccagcctc ccacggacct gagcggtccc agtctccagc tgcctctgac tgctcatcct |
| 841 | catcctcctc ggccagcctg ccttcctccg gcaggagcag cctgggcagt caccagctcc |
| 901 | cgcgggggta catctccatt ccggtgatac acgagcagaa cgttacccgg ccagcagccc |
| 961 | agccctcctt ccaccaagcc cagaagacgc actacccagc gcagcagggg gagtaccaga |
| 1021 | cccaccagcc tgtgtaccac aagatccagg gggatgactg ggagccccgg cccctgcggg |
| 1081 | cggcatcccc gttcaggtca tctgtccagg gtgcatcgag ccgggagggc tcaccagcca |
| 1141 | ggagcagcac gccactccac tccccctcgc ccatccgtgt gcacaccgtg gtcgacaggc |
| 1201 | ctcagcagcc catgacccat cgagaaactg cacctgtttc ccagcctgaa aacaaaccag |
| 1261 | aaagtaagcc aggcccagtt ggaccagaac tccctcctgg acacatccca attcaagtga |
| 1321 | tccgcaaaga ggtggattct aaacctgttt cccagaagcc cccacctccc tctgagaagg |
| 1381 | tagaggtgaa agttccccct gctccagttc cttgtcctcc tcccagccct ggcccttctg |
| 1441 | ctgtcccctc ttcccccaag agtgtggcta cagaagagag ggcagccccc agcactgccc |
| 1501 | ctgcagaagc tacacctcca aaaccaggag aagccgaggc tcccccaaaa catccaggag |
| 1561 | tgctgaaagt ggaagccatc ctggagaagg tacaggggct ggagcaggct gtagacaact |
| 1621 | ttgaaggcaa gaagactgac aaaaagtacc tgatgatcga agagtatttg accaaagagc |
| 1681 | tgctggccct ggattcagtg gaccccgagg gacgagccga tgtgcgtcag gccaggagag |
| 1741 | acggtgtcag gaaggttcag accatcttgg aaaaacttga acagaaagcc attgatgtcc |
| 1801 | caggtcaagt ccaggtctat gaactccagc ccagcaacct tgaagcagat cagccactgc |
| 1861 | aggcaatcat ggagatgggt gccgtggcag cagacaaggg caagaaaaat gctggaaatg |
| 1921 | cagaagatcc ccacacagaa acccagcagc cagaagccac agcagcagcg acttcaaacc |
| 1981 | ccagcagcat gacagacacc cctggtaacc cagcagcacc gtagcctctg ccctgtaaaa |
| 2041 | atcagactcg gaaccgatgt gtgctttagg gaattttaag ttgcatgcat ttcagagact |
| 2101 | ttaagtcagt tggtttttat tagctgcttg gtatgcagta acttgggtgg aggcaaaaca |
| 2161 | ctaataaaag ggctaaaaag gaaaatgatg cttttcttct atattcttac tctgtacaaa |
| 2221 | taaagaagtt gcttgttgtt tgagaagttt aaccccgttg cttgttgttc tgcagccctg |
| 2281 | tctacttggg cacccccacc acctgttagc tgtggttgtg cactgtcttt tgtagctctg |
| 2341 | gactggaggg gtagatgggg agtcaattac ccatcacata aatatgaaac atttatcaga |
| 2401 | aatgttgcca ttttaatgag atgattttct tcatctcata attaaaatac ctgactttag |
| 2461 | agagagtaaa atgtgccagg agccatagga atatctgtat gttggatgac tttaatgcta |
| 2521 | cattttaaaa aaagaaaata aagtaataat ataactcaaa a |

//
SEQ ID NO: 9

| | |
|---|---|
| <210> | 1 |
| <211> | 774 |
| <212> | DNA |
| <213> | Homo sapiens |
| <220> | |
| <221> | misc_feature |
| <223> | hNTF3 |
| <400> | 9 |

| | |
|---|---|
| atgtccatct tgttttatgt gatatttctc gcttatctcc gtggcatcca aggtaacaac | 60 |
| atggatcaaa ggagtttgcc agaagactcg ctcaattccc tcattattaa gctgatccag | 120 |
| gcagatattt tgaaaaacaa gctctccaag cagatggtgg acgttaagga aaattaccag | 180 |
| agcaccctgc ccaaagctga ggctccccga gagccggagc ggggagggcc cgccaagtca | 240 |
| gcattccagc cggtgattgc aatggacacc gaactgetgc gacaacagag acgctacaac | 300 |
| tcaccgcggg toctgctgag cgacagcacc cccttggage ccccgccctt gtatctcatg | 360 |
| gaggattacg tgggcagccc cgtggtggog aacagaacat cacggcggaa acggtacgcg | 420 |
| gagcataaga gtcaccgagg ggagtactcg gtatgtgaca gtgagagtct gtgggtgacc | 480 |
| gacaagtcat cggccatoga cattogggga caccaggtca cggtgctggg ggagatcaaa | 540 |
| acgggcaact ctcccgtcaa acaatatttt tatgaaacgc gatgtaagga agccaggccg | 600 |
| gtcaaaaacg gttgcagggg tattgatgat aaacactgga actctcagtg caaaacatcc | 660 |
| caaacctacg tccgagcact gacttcagag aacaataaac togtgggctg gcggtggata | 720 |
| cggatagaca cgtcctgtgt gtgtgccttg togagaaaaa toggaagaac atga | 774 |

SEQ ID NO: 10

| | |
|---|---|
| <210> | 2 |
| <211> | 270 |
| <212> | PRT |
| <213> | Homo sapiens |
| <220> | |
| <221> | MISC_FEATURE |
| <223> | NT-3 amino acid sequence |
| <400> | 10 |

```
Met Val Thr Phe Ala Thr Ile Leu Gln Val Asn Lys Val Met Ser Ile
1 5 10 15
Leu Phe Tyr Val Ile Phe Leu Ala Tyr Leu Arg Gly Ile Gln Gly Asn
                        20 25 30
Asn Met Asp Gln Arg Ser Leu Pro Glu Asp Ser Leu Asn Ser Leu Ile
                35 40 45
Ile Lys Leu Ile Gln Ala Asp Ile Leu Lys Asn Lys Leu Ser Lys Gln
        50 55 60
Met Val Asp Val Lys Glu Asn Tyr Gln Ser Thr Leu Pro Lys Ala Glu
65 70 75 80
Ala Pro Arg Glu Pro Glu Arg Gly Gly Pro Ala Lys Ser Ala Phe Gln
                                85 90 95
Pro Val Ile Ala Met Asp Thr Glu Leu Leu Arg Gln Gln Arg Arg Tyr
                        100 105 110
Asn Ser Pro Arg Val Leu Leu Ser Asp Ser Thr Pro Leu Glu Pro Pro
                115 120 125
Pro Leu Tyr Leu Met Glu Asp Tyr Val Gly Ser Pro Val Val Ala Asn
        130 135 140
Arg Thr Ser Arg Arg Lys Arg Tyr Ala Glu His Lys Ser His Arg Gly
145 150 155 160
Glu Tyr Ser Val Cys Asp Ser Glu Ser Leu Trp Val Thr Asp Lys Ser
                                165 170 175
Ser Ala Ile Asp Ile Arg Gly His Gln Val Thr Val Leu Gly Glu Ile
                        180 185 190
Lys Thr Gly Asn Ser Pro Val Lys Gln Tyr Phe Tyr Glu Thr Arg Cys
                195 200 205
Lys Glu Ala Arg Pro Val Lys Asn Gly Cys Arg Gly Ile Asp Asp Lys
        210 215 220
His Trp Asn Ser Gln Cys Lys Thr Ser Gln Thr Tyr Val Arg Ala Leu
225 230 235 240
Thr Ser Glu Asn Asn Lys Leu Val Gly Trp Arg Trp Ile Arg Ile Asp
                                245 250 255
Thr Ser Cys Val Cys Ala Leu Ser Arg Lys Ile Gly Arg Thr
                        260 265 270
SEQ ID NO: 11
```

| | |
|---|---|
| <210> | 11 |
| <211> | 714 |
| <212> | DNA |
| <213> | Artificial Sequence |
| <220> | |
| <223> | Synthetic Polynucleotide |
| <220> | |
| <221> | misc_feature |
| <223> | tMCK Promoter |
| <400> | 11 |

| | |
|---|---|
| ccactacggg tctaggctgc ccatgtaagg aggcaaggcc tggggacacc cgagatgcct | 60 |
| ggttataatt aaccccaaca cctgctgccc cccccccccc aacacctgct gcctgagcct | 120 |
| gagcggttac cccaccccgg tgcctgggtc ttaggctctg tacaccatgg aggagaagct | 180 |
| cgctctaaaa ataaccctgt ccctggtgga tccactacgg gtctatgctg cccatgtaag | 240 |
| gaggcaaggc ctggggacac ccgagatgcc tggttataat taaccccaac acctgctgcc | 300 |
| cccccccccc caacacctgc tgcctgagcc tgagcggtta ccccaccccg gtgcctgggt | 360 |
| cttaggctct gtacaccatg gaggagaagc tcgctctaaa aataaccctg tccctggtgg | 420 |
| accactacgg gtctaggctg cccatgtaag gaggcaaggc ctggggacac ccgagatgcc | 480 |
| tggttataat taaccccaac acctgctgcc cccccccccc aacacctgct gcctgagcct | 540 |
| gagcggttac cccaccccgg tgcctgggtc ttaggctctg tacaccatgg aggagaagct | 600 |
| cgctctaaaa ataaccctgt ccctggtcct ccctggggac agcccctcct ggctagtcac | 660 |
| accctgtagg ctcctctata taacccaggg gcacaggggc tgcccccggg tcac | 714 |

SEQ ID NO: 12
<210> 3
<211> 5884
<212> DNA
<213> Artificial Sequence
<220>
<223> Synthetic Polynucleotide
<220>
<221> misc_feature
<223> sc pAAV.tMCK.NTF3 plasmid genome full Sequence
<400> 12

| | |
|---|---|
| cagcagctgc gcgctcgctc gctcactgag gccgcccggg caaagcccgg gcgtcgggcg | 60 |
| acctttggtc gcccggcctc agtgagcgag cgagcgcgca gagagggagt ggggttaacc | 120 |
| aattggcggc cgcaaacttg catgccccac tacgggtcta ggctgcccat gtaaggaggc | 180 |
| aaggcctggg gacacccgag atgcctggtt ataattaacc ccaacacctg ctgccccccc | 240 |
| ccccccaaca cctgctgcct gagcctgagc ggttacccca ccccggtgcc tgggtcttag | 300 |
| gctctgtaca ccatggagga gaagctcgct ctaaaaataa ccctgtccct ggtggatcca | 360 |
| ctacgggtct atgctgccca tgtaaggagg caaggcctgg ggacacccga gatgcctggt | 420 |
| tataattaac cccaacacct gctgcccccc cccccccaac acctgctgcc tgagcctgag | 480 |
| cggttacccc accccggtgc ctgggtctta ggctctgtac accatggagg agaagctcgc | 540 |
| tctaaaaata accctgtccc tggtggacca ctacgggtct aggctgccca tgtaaggagg | 600 |
| caaggcctgg ggacacccga gatgcctggt tataattaac cccaacacct gctgcccccc | 660 |
| ccccccaaca cctgctgcct gagcctgagc ggttacccca ccccggtgcc tgggtcttag | 720 |
| gctctgtaca ccatggagga gaagctcgct ctaaaaataa ccctgtccct ggtcctccct | 780 |
| ggggacagcc cctcctggct agtcacaccc tgtaggctcc tctatataac ccaggggcac | 840 |
| aggggctgcc cccgggtcac ctgcagaagt tggtcgtgag gcactgggca ggtaagtatc | 900 |
| aaggttacaa gacaggttta aggagaccaa tagaaactgg gcttgtcgag acagagaaga | 960 |
| ctcttgcgtt tctgataggc acctattggt cttactgaca tccactttgc ctttctctcc | 1020 |
| acaggtgtcc actcccagtt caattacagc gcgtggtacc tgcagggata tccaccatgt | 1080 |
| ccatcttgtt ttatgtgata tttctcgctt atctccgtgg catccaaggt aacaacatgg | 1140 |
| atcaaaggag tttgccagaa gactcgctca attccctcat tattaagctg atccaggcag | 1200 |
| atattttgaa aaacaagctc tccaagcaga tggtggacgt taaggaaaat taccagagca | 1260 |
| ccctgcccaa agctgaggct ccccgagagc cggagcgggg agggcccgcc aagtcagcat | 1320 |
| tccagccggt gattgcaatg gacaccgaac tgctgcgaca acagagacgc tacaactcac | 1380 |
| cgcgggtcct gctgagcgac agcaccccct tggagccccc gcccttgtat ctcatggagg | 1440 |
| attacgtggg cagccccgtg gtggcgaaca gaacatcacg gcggaaacgg tacgcggagc | 1500 |
| ataagagtca ccgaggggag tactcggtat gtgacagtga gagtctgtgg gtgaccgaca | 1560 |
| agtcatcggc catcgacatt cggggacacc aggtcacggt gctgggggag atcaaaacgg | 1620 |
| gcaactctcc cgtcaaacaa tatttttatg aaacgcgatg taaggaagcc aggccggtca | 1680 |
| aaaacggttg caggggtatt gatgataaac actggaactc tcagtgcaaa acatcccaaa | 1740 |
| cctacgtccg agcactgact tcagagaaca ataaactcgt gggctggcgg tggatacgga | 1800 |
| tagacacgtc ctgtgtgtgt gccttgtcga gaaaaatcgg aagaacatga ggcggccgcg | 1860 |
| gggatccaga catgataaga tacattgatg agtttggaca aaccacaact agaatgcagt | 1920 |
| gaaaaaaatg ctttatttgt gaaatttgtg atgctattgc tttatttgta accattataa | 1980 |
| gctgcaataa acaagttaac aacaacaatt gcattcattt tatgtttcag gttcaggggg | 2040 |
| aggtgtggga ggttttttcg gcgcgcctct agagcatggc tacgtagata agtagcatgg | 2100 |
| cgggttaatc attaactaca aggaacccct agtgatggag ttggccactc cctctctgcg | 2160 |
| cgctcgctcg ctcactgagg ccgggcgacc aaaggtcgcc cgacgcccgg gctttgcccg | 2220 |
| ggcggcctca gtgagcgagc gagcgcgcca gctggcgtaa tagcgaagag gcccgcaccg | 2280 |
| atcgcccttc ccaacagttg cgcagcctga atggcgaatg gaattccaga cgattgagcg | 2340 |
| tcaaaatgta ggtatttcca tgagcgtttt tcctgttgca atggctggcg gtaatattgt | 2400 |
| tctggatatt accagcaagg ccgatagttt gagttcttct actcaggcaa gtgatgttat | 2460 |
| tactaatcaa agaagtattg cgacaacggt taatttgcgt gatggacaga ctcttttact | 2520 |
| cggtggcctc actgattata aaaacacttc tcaggattct ggcgtaccgt tcctgtctaa | 2580 |
| aatcccttta atcggcctcc tgtttagctc ccgctctgat tctaacgagg aaagcacgtt | 2640 |
| atacgtgctc gtcaaagcaa ccatagtacg cgccctgtag cggcgcatta agcgcggcgg | 2700 |
| gtgtggtggt tacgcgcagc gtgaccgcta cacttgccag cgccctagcg cccgctcctt | 2760 |
| tcgctttctt cccttccttt ctcgccacgt tcgccggctt tccccgtcaa gctctaaatc | 2820 |
| gggggctccc tttagggttc cgatttagtg ctttacggca cctcgacccc aaaaaacttg | 2880 |
| attagggtga tggttcacgt agtgggccat cgccctgata gacggttttt cgccctttga | 2940 |
| cgttggagtc cacgttcttt aatagtggac tcttgttcca aactggaaca acactcaacc | 3000 |
| ctatctcggt ctattctttt gatttataag ggattttgcc gatttcggcc tattggttaa | 3060 |
| aaaatgagct gatttaacaa aaatttaacg cgaattttaa caaaatatta acgtttacaa | 3120 |
| tttaaatatt tgcttataca atcttcctgt ttttggggct tttctgatta tcaaccgggg | 3180 |
| tacatatgat tgacatgcta gttttacgat taccgttcat cgattctctt gtttgctcca | 3240 |
| gactctcagg caatgacctg atagcctttg tagagacctc tcaaaaatag ctaccctctc | 3300 |
| cggcatgaat ttatcagcta gaacggttga atatcatatt gatggtgatt tgactgtctc | 3360 |
| cggcctttct cacccgtttg aatctttacc tacacattac tcaggcattg catttaaaat | 3420 |
| atatgagggt tctaaaaatt tttatccttg cgttgaaata aaggcttctc ccgcaaaagt | 3480 |
| attacagggt cataatgttt ttggtacaac cgatttagct ttatgctctg aggctttatt | 3540 |
| gcttaatttt gctaattctt tgccttgcct gtatgattta ttggatgttg gaattcctga | 3600 |
| tgcggtattt tctccttacg catctgtgcg gtatttcaca ccgcatatgg tgcactctca | 3660 |
| gtacaatctg ctctgatgcc gcatagttaa gccagccccg acacccgcca acacccgctg | 3720 |
| acgcgccctg acgggcttgt ctgctcccgg catccgctta cagacaagct gtgaccgtct | 3780 |
| ccgggagctg catgtgtcag aggttttcac cgtcatcacc gaaacgcgcg agacgaaagg | 3840 |
| gcctcgtgat acgcctattt ttataggtta atgtcatgat aataatggtt tcttagacgt | 3900 |
| caggtggcac ttttcgggga aatgtgcgcg gaacccctat ttgtttattt ttctaaatac | 3960 |
| attcaaatat gtatccgctc atgagacaat aaccctgata aatgcttcaa taatattgaa | 4020 |
| aaaggaagag tatgagtatt caacatttcc gtgtcgccct tattcccttt tttgcggcat | 4080 |
| tttgccttcc tgtttttgct cacccagaaa cgctggtgaa agtaaaagat gctgaagatc | 4140 |
| agttgggtgc acgagtgggt tacatcgaac tggatctcaa cagcggtaag atccttgaga | 4200 |
| gttttcgccc cgaagaacgt tttccaatga tgagcacttt taaagttctg ctatgtggcg | 4260 |
| cggtattatc ccgtattgac gccgggcaag agcaactcgg tcgccgcata cactattctc | 4320 |
| agaatgactt ggttgagtac tcaccagtca cagaaaagca tcttacggat ggcatgacag | 4380 |
| taagagaatt atgcagtgct gccataacca tgagtgataa cactgcggcc aacttacttc | 4440 |
| tgacaacgat cggaggaccg aaggagctaa ccgctttttt gcacaacatg ggggatcatg | 4500 |
| taactcgcct tgatcgttgg gaaccggagc tgaatgaagc cataccaaac gacgagcgtg | 4560 |
| acaccacgat gcctgtagca atggcaacaa cgttgcgcaa actattaact ggcgaactac | 4620 |
| ttactctagc ttcccggcaa caattaatag actggatgga ggcggataaa gttgcaggac | 4680 |
| cacttctgcg ctcggccctt ccggctggct ggtttattgc tgataaatct ggagccggtg | 4740 |
| agcgtgggtc tcgcggtatc attgcagcac tggggccaga tggtaagccc tcccgtatcg | 4800 |
| tagttatcta cacgacgggg agtcaggcaa ctatggatga acgaaataga cagatcgctg | 4860 |
| agataggtgc ctcactgatt aagcattggt aactgtcaga ccaagtttac tcatatatac | 4920 |
| tttagattga tttaaaactt catttttaat ttaaaaggat ctaggtgaag atcctttttg | 4980 |
| ataatctcat gaccaaaatc ccttaacgtg agttttcgtt ccactgagcg tcagaccccg | 5040 |
| tagaaaagat caaaggatct tcttgagatc ctttttttct gcgcgtaatc tgctgcttgc | 5100 |
| aaacaaaaaa accaccgcta ccagcggtgg tttgtttgcc ggatcaagag ctaccaactc | 5160 |
| tttttccgaa ggtaactggc ttcagcagag cgcagatacc aaatactgtc cttctagtgt | 5220 |
| agccgtagtt aggccaccac ttcaagaact ctgtagcacc gcctacatac ctcgctctgc | 5280 |
| taatcctgtt accagtggct gctgccagtg gcgataagtc gtgtcttacc gggttggact | 5340 |
| caagacgata gttaccggat aaggcgcagc ggtcgggctg aacggggggt tcgtgcacac | 5400 |
| agcccagctt ggagcgaacg acctacaccg aactgagata cctacagcgt gagctatgag | 5460 |
| aaagcgccac gcttcccgaa gggagaaagg cggacaggta tccggtaagc ggcagggtcg | 5520 |
| gaacaggaga gcgcacgagg gagcttccag ggggaaacgc ctggtatctt tatagtcctg | 5580 |
| tcgggtttcg ccacctctga cttgagcgtc gatttttgtg atgctcgtca ggggggcgga | 5640 |
| gcctatggaa aaacgccagc aacgcggcct ttttacggtt cctggccttt tgctggcctt | 5700 |
| ttgctcacat gttctttcct gcgttatccc ctgattctgt ggataaccgt attaccgcct | 5760 |
| ttgagtgagc tgataccgct cgccgcagcc gaacgaccga gcgcagcgag tcagtgagcg | 5820 |
| aggaagcgga agagcgccca atacgcaaac cgcctctccc cgcgcgttgg ccgattcatt | 5880 |
| aatg | 5884 |

SEQ ID NO: 13 ss.pAAV.CMV.hBAG3

| | |
|---|---|
| 1 | GCGCGCTCGC TCGCTCACTG AGGCCGCCCG GGCAAAGCCC GGGCGTCGGG CGACCTTTGG |
| 61 | TCGCCCGGCC TCAGTGAGCG AGCGAGCGCG CAGAGAGGGA GTGGCCAACT CCATCACTAG |
| 121 | GGGTTCCTTG TAGTTAATGA TTAACCCGCC ATGCTACTTA TCTACGTAGC CATGCTCTAG |
| 181 | GTCGTTACAT AACTTACGGT AAATGGCCCG CCTGGCTGAC CGCCCAACGA CCCCCGCCCA |
| 241 | TTGACGTCAA TAATGACGTA TGTTCCCATA GTAACGCCAA TAGGGACTTT CCATTGACGT |
| 301 | CAATGGGTGG AGTATTTACG GTAAACTGCC CACTTGGCAG TACATCAAGT GTATCATATG |
| 361 | CCAAGTACGC CCCCTATTGA CGTCAATGAC GGTAAATGGC CCGCCTGGCA TTATGCCCAG |
| 421 | TACATGACCT TATGGGACTT TCCTACTTGG CAGTACATCT ACGTATTAGT CATCGCTATT |
| 481 | ACCATGGTGA TGCGGTTTTG GCAGTACATC AATGGGCGTG GATAGCGGTT TGACTCACGG |
| 541 | GGATTTCCAA GTCTCCACCC CATTGACGTC AATGGGAGTT TGTTTTGGCA CCAAAATCAA |
| 601 | CGGGACTTTC CAAAATGTCG TAACAACTCC GCCCCATTGA CGCAAATGGG CGGTAGGCGT |
| 661 | GTACGGTGGG AGGTCTATAT AAGCAGAGCT CGTTTAGTGA ACCGTCAGAT CGCCTGGAGA |
| 721 | CGCCATCCAC GCTGTTTTGA CCTCCATAGA AGACACCGGG ACCGATCCAG CCTCCGGACT |
| 781 | CTAGAGGATC CGGTACTCGA GGAACTGAAA AACCAGAAAG TTAACTGGTA AGTTTAGTCT |
| 841 | TTTTGTCTTT TATTTCAGGT CCCGGATCCG GTGGTGGTGC AAATCAAAGA ACTGCTCCTC |
| 901 | AGTGGATGTT GCCTTTACTT CTAGGCCTGT ACGGAAGTGT TACTTCTGCT CTAAAAGCTG |
| 961 | CGGAATTGTA CCCGCGGCCG CCACCATGAG CGCCGCCACC CACTCGCCCA TGATGCAGGT |
| 1021 | GGCGTCCGGC AACGGTGACC GCGACCCTTT GCCCCCCGGA TGGGAGATCA AGATCGACCC |
| 1081 | GCAGACCGGC TGGCCCTTCT TCGTGGACCA CAACAGCCGC ACCACTACGT GGAACGACCC |
| 1141 | GCGCGTGCCC TCTGAGGGCC CCAAGGAGAC TCCATCCTCT GCCAATGGCC CTTCCCGGGA |
| 1201 | GGGCTCTAGG CTGCCGCCTG CTAGGGAAGG CCACCCTGTG TACCCCCAGC TCCGACCAGG |
| 1261 | CTACATTCCC ATTCCTGTGC TCCATGAAGG CGCTGAGAAC CGGCAGGTGC ACCCTTTCCA |
| 1321 | TGTCTATCCC CAGCCTGGGA TGCAGCGATT CCGAACTGAG GCGGCAGCAG CGGCTCCTCA |
| 1381 | GAGGTCCCAG TCACCTCTGC GGGGCATGCC AGAAACCACT CAGCCAGATA AACAGTGTGG |
| 1441 | ACAGGTGGCA GCGGCGGCGG CAGCCCAGCC CCCAGCCTCC CACGGACCTG AGCGGTCCCA |
| 1501 | GTCTCCAGCT GCCTCTGACT GCTCATCCTC ATCCTCCTCG GCCAGCCTGC CTTCCTCCGG |
| 1561 | CAGGAGCAGC CTGGGCAGTC ACCAGCTCCC GCGGGGGTAC ATCTCCATTC CGGTGATACA |
| 1621 | CGAGCAGAAC GTTACCCGGC CAGCAGCCCA GCCCTCCTTC CACCAAGCCC AGAAGACGCA |
| 1681 | CTACCCAGCG CAGCAGGGGG AGTACCAGAC CCACCAGCCT GTGTACCACA AGATCCAGGG |
| 1741 | GGATGACTGG GAGCCCCGGC CCCTGCGGGC GGCATCCCCG TTCAGGTCAT CTGTCCAGGG |
| 1801 | TGCATCGAGC CGGGAGGGCT CACCAGCCAG GAGCAGCACG CCACTCCACT CCCCCTCGCC |
| 1861 | CATCCGTGTG CACACCGTGG TCGACAGGCC TCAGCAGCCC ATGACCCATC GAGAAACTGC |
| 1921 | ACCTGTTTCC CAGCCTGAAA ACAAACCAGA AAGTAAGCCA GGCCCAGTTG GACCAGAACT |
| 1981 | CCCTCCTGGA CACATCCCAA TTCAAGTGAT CCGCAAAGAG GTGGATTCTA AACCTGTTTC |
| 2041 | CCAGAAGCCC CCACCTCCCT CTGAGAAGGT AGAGGTGAAA GTTCCCCCTG CTCCAGTTCC |
| 2101 | TTGTCCTCCT CCCAGCCCTG GCCCTTCTGC TGTCCCCTCT TCCCCCAAGA GTGTGGCTAC |
| 2161 | AGAAGAGAGG GCAGCCCCCA GCACTGCCCC TGCAGAAGCT ACACCTCCAA AACCAGGAGA |
| 2221 | AGCCGAGGCT CCCCCAAAAC ATCCAGGAGT GCTGAAAGTG GAAGCCATCC TGGAGAAGGT |
| 2281 | GCAGGGGCTG GAGCAGGCTG TAGACAACTT TGAAGGCAAG AAGACTGACA AAAAGTACCT |
| 2341 | GATGATCGAA GAGTATTTGA CCAAAGAGCT GCTGGCCCTG GATTCAGTGG ACCCCGAGGG |
| 2401 | ACGAGCCGAT GTGCGTCAGG CCAGGAGAGA CGGTGTCAGG AAGGTTCAGA CCATCTTGGA |
| 2461 | AAAACTTGAA CAGAAAGCCA TTGATGTCCC AGGTCAAGTC CAGGTCTATG AACTCCAGCC |
| 2521 | CAGCAACCTT GAAGCAGATC AGCCACTGCA GGCAATCATG GAGATGGGTG CCGTGGCAGC |
| 2581 | AGACAAGGGC AAGAAAAATG CTGGAAATGC AGAAGATCCC CACACAGAAA CCCAGCAGCC |
| 2641 | AGAAGCCACA GCAGCAGCGA CTTCAAACCC CAGCAGCATG ACAGACACCC CTGGTAACCC |
| 2701 | AGCAGCACCG TGAGGCGGCC GCGGGGATCC AGACATGATA AGATACATTG ATGAGTTTGG |
| 2761 | ACAAACCACA ACTAGAATGC AGTGAAAAAA ATGCTTTATT TGTAAAATTT GTGATGCTAT |
| 2821 | TGCTTTATTT GTAACCATTA TAAGCTGCAA TAAACAAGTT AACAACAACA ATTGCATTCA |
| 2881 | TTTTATGTTT CAGGTTCAGG GGGAGGTGTG GGAGGTTTTT TCGGATCCTC TAGAGTCGAC |
| 2941 | CAGAGCATGG CTACGTAGAT AAGTAGCATG GCGGGTTAAT CATTAACTAC AAGGAACCCC |
| 3001 | TAGTGATGGA GTTGGCCACT CCCTCTCTGC GCGCTCGCTC GCTCACTGAG GCCGGGCGAC |
| 3061 | CAAAGGTCGC CCGACGCCCG GGCTTTGCCC GGGCGGCCTC AGTGAGCGAG CGAGCGCGCA |
| 3121 | GCTGCATTAA TGAATCGGCC AACGCGCGGG GAGAGGCGGT TTGCGTATTG GGCGCTCTTC |
| 3181 | CGCTTCCTCG CTCACTGACT CGCTGCGCTC GGTCGTTCGG CTGCGGCGAG CGGTATCAGC |
| 3241 | TCACTCAAAG GCGGTAATAC GGTTATCCAC AGAATCAGGG GATAACGCAG GAAAGAACAT |
| 3301 | GTGAGCAAAA GGCCAGCAAA AGGCCAGGAA CCGTAAAAAG GCCGCGTTGC TGGCGTTTTT |
| 3361 | CCATAGGCTC CGCCCCCCTG ACGAGCATCA CAAAAATCGA CGCTCAAGTC AGAGGTGGCG |
| 3421 | AAACCCGACA GGACTATAAA GATACCAGGC GTTTCCCCCT GGAAGCTCCC TCGTGCGCTC |
| 3481 | TCCTGTTCCG ACCCTGCCGC TTACCGGATA CCTGTCCGCC TTTCTCCCTT CGGGAAGCGT |
| 3541 | GGCGCTTTCT CATAGCTCAC GCTGTAGGTA TCTCAGTTCG GTGTAGGTCG TTCGCTCCAA |
| 3601 | GCTGGGCTGT GTGCACGAAC CCCCCGTTCA GCCCGACCGC TGCGCCTTAT CCGGTAACTA |
| 3661 | TCGTCTTGAG TCCAACCCGG TAAGACACGA CTTATCGCCA CIGGCAGCAG CCACTGGTAA |
| 3721 | CAGGATTAGC AGAGCGAGGT ATGTACGCGG TGCTACAGAG TTCTTGAAGT GGTGGCCTAA |
| 3781 | CTACGGCTAC ACTAGAAGGA CAGTATTTGG TATCTGCGCT CTGCTGAAGC CAGTTACCTT |
| 3841 | CGGAAAAAGA GTTGGTAGCT CTTGATCCGG CAAACAAACC ACCGCTGGTA GCGGTGGTTT |
| 3901 | TTTTGTTTGC AAGCAGCAGA TTACGCGCAG AAAAAAAGGA TCTCAAGAAG ATCCTTTGAT |
| 3961 | CTTTTCTACG GGGTCTGACG CTCAGTGGAA CGAAAACTCA CGTTAAGGGA TTTTGGTCAT |
| 4021 | GAGATTATCA AAAAGGATCT TCACCTAGAT CCTTTTAAAT TAAAAATGAA GTTTTAAATC |
| 4081 | AATCTAAAGT ATATATGAGT AAACTTGGTC TGACAGTTAC CAATGCTTAA TCAGTGAGGC |
| 4141 | ACCTATCTCA GCGATCTGTC TATTTCGTTC ATCCATAGTT GCCTGACTCC CCGTCGTGTA |
| 4201 | GATAACTACG ATACGGGAGG GCTTACCATC TGGCCCCAGT GCTGCAATGA TACCGCGAGA |
| 4261 | CCCACGCTCA CCGGCTCCAG ATTTATCAGC AATAAACCAG CCAGCCGGAA GGGCCGAGCG |
| 4321 | CAGAAGTGGT CCTGCAACTT TATCCGCCTC CATCCAGTCT ATTAATTGTT GCCGGGAAGC |
| 4381 | TAGAGTAAGT AGTTCGCCAG TTAATAGTTT GCGCAACGTT GTTGCCATTG CTACAGGCAT |
| 4441 | CGTGGTGTCA CGCTCGTCGT TTGGTATGGC TTCATTCAGC TCCGGTTCCC AACGATCAAG |
| 4501 | GCGAGTTACA TGATCCCCCA TGTTGTGCAA AAAAGCGGTT AGCTCCTTCG GTCCTCCGAT |
| 4561 | CGTTGTCAGA AGTAAGTTGG CCGCAGTGTT ATCACTCATG GTTATGGCAG CACTGCATAA |
| 4621 | TTCTCTTACT GTCATGCCAT CCGTAAGATG CTTTTCTGTG ACTGGTGAGT ACTCAACCAA |
| 4681 | GTCATTCTGA GAATAGTGTA TGCGGCGACC GAGTTGCTCT TGCCCGGCGT CAATACGGGA |
| 4741 | TAATACCGCG CCACATAGCA GAACTTTAAA AGTGCTCATC ATTGGAAAAC GTTCTTCGGG |
| 4801 | GCGAAAACTC TCAAGGATCT TACCGCTGTT GAGATCCAGT TCGATGTAAC CCACTCGTGC |
| 4861 | ACCCAACTGA TCTTCAGCAT CTTTTACTTT CACCAGCGTT TCTGGGTGAG CAAAAACAGG |
| 4921 | AAGGCAAAAT GCCGCAAAAA AGGGAATAAG GGCGACACGG AAATGTTGAA TACTCATACT |
| 4981 | CTTCCTTTTT CAATATTATT GAAGCATTTA TCAGGGTTAT TGTCTCATGA GCGGATACAT |
| 5041 | ATTTGAATGT ATTTAGAAAA ATAAACAAAT AGGGGTTCCG CGCACATTTC CCCGAAAAGT |
| 5101 | GCCACCTGAC GTCTAAGAAA CCATTATTAT CATGACATTA ACCTATAAAA ATAGGCGTAT |
| 5161 | CACGAGGCCC TTTCGTCTCG CGCGTTTCGG TGATGACGGT GAAAACCTCT GACACATGCA |
| 5221 | GCTCCCGGAG ACGGTCACAG CTTGTCTGTA AGCGGATGCC GGGAGCAGAC AAGCCCGTCA |
| 5281 | GGGCGCGTCA GCGGGTGTTG GCGGGTGTCG GGGCTGGCTT AACTATGCGG CATCAGAGCA |
| 5341 | GATTGTACTG AGAGTGCACC ATATGCGGTG TGAAATACCG CACAGATGCG TAAGGAGAAA |
| 5401 | ATACCGCATC AGGAACTTCC AACATCCAAT AAATCATACA GGCAAGGCAA AGAATTAGCA |
| 5461 | AAATTAAGCA ATAAAGCCTC AGAGCATAAA GCTAAATCGG TTGTACCAAA AACATTATGA |
| 5521 | CCCTGTAATA CTTTTGCGGG AGAAGCCTTT ATTTCAACGC AAGGATAAAA ATTTTTAGAA |
| 5581 | CCCTCATATA TTTTAAATGC AATGCCTGAG TAATGTGTAG GTAAAGATTC AAACGGGTGA |
| 5641 | GAAAGGCCGG AGACAGTCAA ATCACCATCA ATATGATATT CAACCGTTCT AGCTGATAAA |
| 5701 | TTCATGCCGG AGAGGGTAGC TATTTTTGAG AGGTCTCTAC AAAGGCTATC AGGTCATTGC |
| 5761 | CTGAGAGTCT GGAGCAAACA AGAGAATCGA TGAACGGTAA TCGTAAAACT AGCATGTCAA |
| 5821 | TCATATGTAC CCCGGTTGAT AATCAGAAAA GCCCCAAAAA CAGGAAGATT GTATAAGCAA |
| 5881 | ATATTTAAAT TGTAAACGTT AATATTTTGT TAAAATTCGC GTTAAATTTT TGTTAAATCA |
| 5941 | GCTCATTTTT TAACCAATAG GCCGAAATCG GCAAAATCCC TTATAAATCA AAAGAATAGA |
| 6001 | CCGAGATAGG GTTGAGTGTT GTTCCAGTTT GGAACAAGAG TCCACTATTA AAGAACGTGG |
| 6061 | ACTCCAACGT CAAAGGGCGA AAAACCGTCT ATCAGGGCGA TGGCCCACTA CGTGAACCAT |
| 6121 | CACCCTAATC AAGTTTTTTG GGGTCGAGGT GCCGTAAATC ACTAAATCGG AACCCTAAAG |
| 6181 | GGAGCCCCCG ATTTAGAGCT TGACGGGGAA AGCCGGCGAA CGTGGCGAGA AAGGAAGGGA |
| 6241 | AGAAAGCGAA AGGAGCGGGC GCTAGGGCGC TGGCAAGTGT AGCGGTCACG CTGCGCGTAA |
| 6301 | CCACCACACC CGCCGCGCTT AATGCGCCGC TACAGGGCGC GTACTATGGT TGCTTTGACG |
| 6361 | AGCACGTATA ACGTGCTTTC CTCGTTAGAA TCAGAGCGGG AGCTAAACAG GAGGCCGATT |
| 6421 | AAAGGGATTT TAGACAGGAA CGGTACGCCA GAATCCTGAG AAGTGTTTTT ATAATCAGTG |
| 6481 | AGGCCACCGA GTAAAAGAGT CTGTCCATCA CGCAAATTAA CCGTTGTCGC AATACTTCTT |
| 6541 | TGATTAGTAA TAACATCACT TGCCTGAGTA GAAGAACTCA AACTATCGGC CTTGCTGGTA |
| 6601 | ATATCCAGAA CAATATTACC GCCAGCCATT GCAACGGAAT CGCCATTCGC CATTCAGGCT |
| 6661 | GCGCAACTGT TGGGAAGGGC GATCGGTGCG GGCCTCTTCG CTATTACGCC AGCT |
| // | |

1. A polynucleotide sequence comprising a transcriptional control element; and nucleotide sequence encoding the human Bcl-associated athanogene 3 protein.

2. The polynucleotide sequence of claim 1, wherein the nucleotide sequence encoding the human Bcl-associated athanogene 3 protein comprises
(a) a nucleotide sequence having at least 90% sequence identity to nucleotides 1112-2839 of SEQ ID NO: 1; or
(b) the nucleotide sequence comprising nucleotides 1112-2839 of SEQ ID NO: 1,
(c) a nucleotide sequence having at least 90% sequence identity to nucleotides 981-2708 of SEQ ID NO: 2,
(d) a nucleotide sequence comprising nucleotides 981-2708 of SEQ ID NO: 2, or
(e) a nucleotide sequence encoding the amino acid sequence of SEQ ID NO: 8.

3. The polynucleotide sequence of claim 1 or 2 wherein the transcriptional control element is a muscle-specific control element.

4. The polynucleotide sequence of claim 3, wherein the muscle-specific control comprises one or more of a human skeletal actin gene element, a cardiac actin gene element, a desmin promoter, a skeletal alpha-actin (ASKA) promoter, a troponin I (TNNI2) promoter, a myocyte-specific enhancer binding factor MEF binding element, a muscle creatine kinase (MCK) promoter, a truncated MCK (tMCK) promoter, a myosin heavy chain (MHC) promoter, a hybrid a-myosin heavy chain enhancer-/MHC enhancer-promoter (MHCK7) promoter, a C5-12 promoter, a murine creatine kinase enhancer element, a skeletal fast-twitch troponin C gene element, a slow-twitch cardiac troponin c gene element, a slow-twitch troponin I gene element, hypoxia-inducible nuclear factor (HIF)-response element (HRE), a steroid-inducible element, and a glucocorticoid response element (GRE).

5. The nucleic acid molecule of claim 1 or 2, wherein the transcriptional control element is a neuronal-specific control element.

6. The nucleic acid molecule of claim 5, wherein the neuronal-specific control element comprises one or more of a platelet-derived growth factor B-chain (PDGFB) promoter, synapsin-1 (Syn) promoter, synapsin-2 promoter, tyrosine hydroxylase promoter, dopamine β-hydroxylase (DBH) promoter, hypoxanthine-guanine phosphoribosyltransferase (HPRT) promoter, low-affinity nerve growth factor receptor (LNGFR) promoter, Calcitonin Gene-Related Peptide promoter (CGRP promoter), Choline Acetyl Transferase (ChAT) promoter, Neuron Specific Enolase (NSE) promoter, Calcium/Calmodulin Dependent Protein Kinase II (CaMKII) promoter, methyl CpG binding protein 2 (MeCP2) promoter, Glial fibrillary acidic protein (GFAP) promoter, Calbindin 2 promoter, Motor neuron and pancreas homeobox 1 (MNX1) promoter also known as the Hb9 promoter, Nestin promoter, Parvalbumin (PVALB) promoter, and the Somatostation (SST) promoter.

7. The polynucleotide sequence of claim 1 or 2 wherein the transcriptional control element comprises the tMCK promoter comprising nucleotides 165-884 of SEQ ID NO: 1.

8. The polynucleotide sequence of any one of claims 1-7, further comprising a CMV enhancer comprising nucleotides 209-463 of SEQ ID NO: 2.

9. The polynucleotide sequence of any one of claims 1-8, further comprising a CBA promoter sequence comprising nucleotides 495-749 of SEQ ID NO: 2.

10. The polynucleotide sequence of any one of claims 1-9, further comprising a chimeric intron comprising nucleotides 937-1069 or a SV40 intron sequence comprising nucleotides 830-926 of SEQ ID NO: 2.

11. The polynucleotide sequence of any one of claims 1-10, further comprising a poly adenylation sequence comprising nucleotides 2849-3048 of SEQ ID NO: 1 or nucleotides 2718-2917 of SEQ ID NO: 2.

12. The polynucleotide sequence of any one of claims 1-11, further comprising one or more inverted terminal repeat sequences (ITRs).

13. A polynucleotide sequence comprising a polynucleotide sequence having or 90% identity to nucleotides 1 to 3245 of SEQ ID NO: 1 or 90% identity to nucleotides 1 to 3114 of SEQ ID NO: 2.

14. A polynucleotide sequence comprising a polynucleotide sequence comprising nucleotides 1 to 3245 of SEQ ID NO: 1 or nucleotides 1 to 3114 of SEQ ID NO: 2.

15. A viral vector comprising the polynucleotide sequence of any one of claims 1-14.

16. The viral vector of claim 15, wherein the viral vector is an adeno-associated virus (AAV), adenovirus, lentivirus, retrovirus, poxvirus, baculovirus, herpes simplex virus, vaccinia virus, or a synthetic virus.

17. The viral vector of claim 15 or 16, wherein the viral vector is an AAV.

18. The viral vector of any one of claims 12-14, wherein the AAV is AAV1, AAV2, AAV3, AAV4, AAV5, AAV6, AAV7, AAV8, AAV9, AAV10, AAV11, AAV12, AAV13, AAVanc80, AAVrh.74, AAVrh.8, AAVrh.10, AAV2/1, AAV2/8, AAV2/9, AAVMYO, or a variant thereof.

19. An rAAV particle comprising the AAV of claim 17 or 18.

20. A composition comprising the polynucleotide of any one of claims 1-14, the viral vector of any one of claims 15-18, or the AAV particle of claim 19 and a pharmaceutically acceptable carrier.

21. The composition of claim 20, wherein the composition is formulated for intravenous, or intramuscular delivery.

22. A method for treating or preventing a disease or disorder associated with misfolded protein or protein aggregates in a subject in need thereof, the method comprising administering to the subject an effective amount of the polynucleotide of any one of claims 1-14, the viral vector of any one of claims 15-18, the AAV particle of claim 19 or the composition of claim 20 or 21.

23. A method of reducing accumulation of misfolded or aggregated proteins in a subject suffering from a disease or disorder associated with misfolded protein or protein aggregates, the method comprising administering to the subject an effective amount of the polynucleotide of any one of claims 1-14, the viral vector of any one of claims 15-18, the AAV particle of claim 19 or the composition of claim 20 or 21.

24. Use of the polynucleotide of any one of claims 1-14, the viral vector of any one of claims 15-18, the AAV particle of claim 19 or the composition of claim 20 or 21 for the preparation of a medicament for treating or preventing a disease or disorder associated with misfolded protein or protein aggregates in a subject in need thereof.

25. Use of the polynucleotide of any one of claims 1-14, the viral vector of any one of claims 15-18, the AAV particle of claim 19 or the composition of claim 20 or 21A for the preparation of a medicament for reducing accumulation of misfolded or aggregated proteins in a subject suffering from a disease or disorder associated with misfolded protein or protein aggregates.

26. A composition for treating or preventing a disease or disorder associated with misfolded protein or protein aggregates in a subject in need thereof, wherein the composition comprises the polynucleotide of any one of claims 1-14, the viral vector of any one of claims 15-18, the AAV particle of claim 19 or the composition of claim 20 or 21.

27. A composition for reducing accumulation of misfolded or aggregated proteins in a subject suffering from a disease or disorder associated with misfolded protein or protein aggregates, wherein the composition comprises the polynucleotide of any one of claims 1-14, the viral vector of any one of claims 15-18, the AAV particle of claim 19 or the composition of claim 20 or 21.

28. The method, use or composition of any one of claims 22-27, wherein the disease or disorder associated with misfolded protein or protein aggregates is a protein aggregate myopathy (PAM).

29. The method, use or composition of claim 28, wherein the PAM is a myofibrilar myopathy (MFM) is a desminopathy, an alpha-B crystallinopathy, myotilinopathy (limb girdle muscular dystrophy type 1A (LGMD1A)), a filaminopathy, a BAG3-related myofibrillar myopathy, a ZASPopathy, a HSPB8 myopathy, a reducing body myopathy (RBM), a hereditary myopathy with early respiratory failure, an epidermolysis bullosa simplex with muscular dystrophy, an MFM-actinopathy or limb girdle muscular dystrophy type 1D (LGMD1D).

30. The method, use or composition of claim 29, wherein the misfolded or aggregated protein associated with a myofibrilar myopathy is desmin, Alpha-crystallin B chain, myotilin, filamin C, BAG family molecular chaperone regulator 3 (BAG-3), Z-band alternatively spliced PDZ-motif containing protein, HSPB8, four-and-a-half LIM domain protein 1, titin, plectin, α-actin or DnaJ heat shock protein family (Hsp40) member B6.

31. The method, use or composition of any one of claims of claim 29 or 30, wherein the myofibrilar myopathy is LGMD1A, and wherein the misfolded or aggregated proteins associated with LGMD1A is myotilin.

32. The method, use or composition of any one of claim 22-27, wherein the disease or disorder associated with misfolded protein or protein aggregates is a neurodegenerative disease.

33. The method, use or composition of claim 32, wherein the neurodegenerative disease associated with misfolded proteins or protein aggregates is amyotrophic lateral sclerosis (ALS), transmissible spongiform encephalopathies (prion disease), synucleinopathies, dementia with Lewy bodies (DLB), multiple system atrophy (MSA), tauopathies, Frontotemporal lobar degeneration (FTLD), Frontotemporal dementia (FTD), Sporadic or familial with or without motor-neuron disease (MND), corticobasal degeneration, frontotemporal lobar degeneration with ubiquitin-positive inclusions, Argyrophilic grain disease, Pick's disease, Amyotrophic lateral sclerosis (ALS), Sporadic ALS, Alzheimer's disease (AD, sporadic and familial), Down syndrome, Familial British dementia, Polyglutamine (polyQ) diseases (Huntington's disease (HD), dentatorubral-pallidoluysian atrophy (DRPLA), spinal bulbar muscular atrophy (SBMA), and six spinocerebellar ataxias (SCA1, SCA2, SCA3, SCA6, SCA7, and SCA17)), Hippocampal sclerosis dementia or Parkinson's disease (PD).

34. The method, use or composition of claim 32 or 33, wherein the misfolded protein or protein aggregates is alpha-synuclein, amyloid-beta, mutated huntingtin, tau protein, prion proteins, misfolded superoxide dismutase 1 (SOD1), isled amyloid polypeptide (IAPP), Musashi protein, p53, Fused in sarcoma (FUS), Progranulin, TAR DNA-binding protein 43 (TDP-43), misfolded transthyretin protein (TTR), valosin-containing protein (VCP), NOTCH3 receptor, mutated cystatin C, polyglutamine repeats, serum amyloid A (SAA), mutated gelsolin, misfolded rhodopsin, medin, dipeptide repeat protein, or atrial natriuretic peptide.

35. The method, use or composition of any one of claims 33-35, wherein the neurodegenerative disease is ALS, and wherein the misfolded protein or protein aggregates associated with the ALS is SOD1.

36. A method of treating or preventing inclusion body myositis or multisystem proteinopathy in a subject in need thereof, the method comprising administering to the subject an effective amount of the polynucleotide of any one of claims 1-14, the viral vector of any one of claims 15-18, the AAV particle of claim 19 or the composition of claim 20 or 21.

37. Use of the polynucleotide of any one of claims 1-14, the viral vector of any one of claims 15-18, the AAV particle of claim 19 or the composition of claim 20 or 21 for the preparation of a medicament for treating or preventing inclusion body myositis or multisystem proteinopathy in a subject in need thereof.

38. A composition for treating or preventing inclusion body myositis or multisystem proteinopathy in a subject in need thereof, wherein the composition comprises the polynucleotide of any one of claims 1-14, the viral vector of any one of claims 15-18, the AAV particle of claim 19 or the composition of claim 20 or 21.

39. The method, use or composition of any one of claims 36-38, wherein the inclusion body myositis (IBM) is associated with Paget disease of bone and/or frontotemporal dementia (IBMPFD).

40. The method, use or composition of any one of claims 36-39, wherein the subject has a mutation in the valosin-containing protein (VCP) gene.

41. The method, use or composition of any one of claims 36-39, wherein the subject is suffering from amyotrophic lateral sclerosis or Charcot-Marie-Tooth type 2.

42. The method, use or composition of any one of claims 22-241, further comprising administering a second nucleic acid encoding a NT-3 polypeptide; wherein
a) the nucleic acid comprises a nucleotide sequence that is at least 90% identical to the nucleotide sequence of SEQ ID NO: 9;
b) the nucleic acid comprises the nucleotide sequence of SEQ ID NO: 9;
c) the nucleic acid comprises a nucleotide sequence encoding an amino acid sequence that is at least 90% identical to SEQ ID NO: 10; or
d) the nucleic acid comprises a nucleotide sequence encoding the amino acid sequence of SEQ ID NO: 10.

43. The method, use or composition of claim 36, wherein the nucleic acid encoding the NT-3 polypeptide is operatively linked to a muscle-specific promoter.

44. The method, use or composition of claim 43, wherein the muscle-specific promoter is muscle-specific creatine kinase promoter (MCK).

45. The method, use or composition of claim 44, wherein the muscle creatine kinase promoter has the nucleotide sequence set out in SEQ ID NO: 11.

46. The method, use or composition of any one of claims 42-45, wherein the second nucleic acid encoding the NT-3 is administered using a viral vector.

47. The method, use or composition of claim 40, wherein the viral vector is a recombinant adeno-associated virus (rAAV).

48. The method, use or composition of claim 47, wherein the rAAV capsid serotype is AAV-1, AAV-2, AAV-3, AAV-4, AAV-5, AAV-6, AAV-7, AAV-8, AAV-9, AAV-10, AAV-11, AAV12, AAV13, Anc80, AAV-B1, AAVrh.10, AAVrh.74 or variant thereof.

49. The method, use or composition of claim 48, wherein the rAAV capsid serotype is AAV-1.

50. The method, use or composition of any one of claims 42-49, wherein the second nucleotide sequence comprises a rAAV genome sequence comprises in order from 5′ to 3′:
(i) a first AAV2 inverted terminal repeat sequence (ITR);
(ii) a muscle creatine kinase promoter/enhancer sequence set out in nucleotides 147-860 of SEQ ID NO: 12;
(iii) a nucleotide sequence encoding a human NT-3 polypeptide; and
(iv) a second AAV2 ITR sequence;
wherein the human NT-3 polypeptide has an amino acid sequence that is at least 90% identical to SEQ ID NO:10 or is 100% identical to SEQ ID NO: 10, or is encoded by a nucleotide sequence at least 90% identical to nucleotides 1077-1850 of SEQ ID NO: 12 or 100% identical to nucleotides 1077-1850 of SEQ ID NO: 12.

51. The method, use or composition of claim 50, wherein the nucleic acid sequence further comprises 3′ to said promoter/enhancer, a chimeric intron set out in nucleotides 892-1024 of SEQ ID NO: 12.

52. The method, use or composition of claim 50 or 51, wherein the nucleic acid sequence further comprises 3′ to said nucleotide sequence encoding a human NT-3 polypeptide, a SV40 polyadenylation signal set out in nucleotides 1860-2059 of SEQ ID NO: 12.

53. The method, use or composition of any one of claims 42-52, wherein the second nucleic acid comprises the scAAV1.tMCK.NTF3 rAAV genome that is at least 90% identical to SEQ ID NO: 12.

54. The method, use or composition of any one of claims 42-53, wherein the nucleic acid comprises the scAAV1.tMCK.NTF3 genome is set out in SEQ ID NO: 12.


SEQUENCE LISTING (13 sequences)

SEQ ID NO: 1 - DNA, 6897 bp, Adeno-associated virus
  source 1..6897: mol_type = genomic DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttaatacgactcactatagggccgcaccggtaagcttccactacgggtctaggctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtggatccactacgggtctaggctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtggatccactacgggtctaggctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtggatcctccctggggacagcccctcctggctagtcacaccctgtaggctcctctatataacccaggggcacaggggctgcccccgggtcacggtggatatccctgcaggtaccacgcgctgtaattgaactgggagtggacacctgtggagagaaaggcaaagtggatgtcagtaagaccaataggtgcctatcagaaacgcaagagtcttctctgtctcgacaagcccagtttctattggtctccttaaacctgtcttgtaaccttgatacttacggagagcaactgcataagggctagcctcgagaattcgccaccatgagcgccgccacccactcgcccatgatgcaggtggcgtccggcaacggtgaccgcgaccctttgccccccggatgggagatcaagatcgacccgcagaccggctggcccttcttcgtggaccacaacagccgcaccactacgtggaacgacccgcgcgtgccctctgagggccccaaggagactccatcctctgccaatggcccttcccgggagggctctaggctgccgcctgctagggaaggccaccctgtgtacccccagctccgaccaggctacattcccattcctgtgctccatgaaggcgctgagaaccggcaggtgcaccctttccatgtctatccccagcctgggatgcagcgattccgaactgaggcggcagcagcggctcctcagaggtcccagtcacctctgcggggcatgccagaaaccactcagccagataaacagtgtggacaggtggcagcggcggcggcagcccagcccccagcctcccacggacctgagcggtcccagtctccagctgcctctgactgctcatcctcatcctcctcggccagcctgccttcctccggcaggagcagcctgggcagtcaccagctcccgcgggggtacatctccattccggtgatacacgagcagaacgttacccggccagcagcccagccctccttccaccaagcccagaagacgcactacccagcgcagcagggggagtaccagacccaccagcctgtgtaccacaagatccagggggatgactgggagccccggcccctgcgggcggcatccccgttcaggtcatctgtccagggtgcatcgagccgggagggctcaccagccaggagcagcacgccactccactccccctcgcccatccgtgtgcacaccgtggtcgacaggcctcagcagcccatgacccatcgagaaactgcacctgtttcccagcctgaaaacaaaccagaaagtaagccaggcccagttggaccagaactccctcctggacacatcccaattcaagtgatccgcaaagaggtggattctaaacctgtttcccagaagcccccacctccctctgagaaggtagaggtgaaagttccccctgctccagttccttgtcctcctcccagccctggcccttctgctgtcccctcttcccccaagagtgtggctacagaagagagggcagcccccagcactgcccctgcagaagctacacctccaaaaccaggagaagccgaggctcccccaaaacatccaggagtgctgaaagtggaagccatcctggagaaggtgcaggggctggagcaggctgtagacaactttgaaggcaagaagactgacaaaaagtacctgatgatcgaagagtatttgaccaaagagctgctggccctggattcagtggaccccgagggacgagccgatgtgcgtcaggccaggagagacggtgtcaggaaggttcagaccatcttggaaaaacttgaacagaaagccattgatgtcccaggtcaagtccaggtctatgaactccagcccagcaaccttgaagcagatcagccactgcaggcaatcatggagatgggtgccgtggcagcagacaagggcaagaaaaatgctggaaatgcagaagatccccacacagaaacccagcagccagaagccacagcagcagcgacttcaaaccccagcagcatgacagacacccctggtaacccagcagcaccgtgaggcggccgcggggatccagacatgataagatacattgatgagtttggacaaaccacaactagaatgcagtgaaaaaaatgctttatttgtaaaatttgtgatgctattgctttatttgtaaccattataagctgcaataaacaagttaacaacaacaattgcattcattttatgtttcaggttcagggggaggtgtgggaggttttttcggatcctctagagtcgaccagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtacgcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaaaatattccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaaccggtaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaacttccaacatccaataaatcatacaggcaaggcaaagaattagcaaaattaagcaataaagcctcagagcataaagctaaatcggttgtaccaaaaacattatgaccctgtaatacttttgcgggagaagcctttatttcaacgcaaggataaaaatttttagaaccctcatatattttaaatgcaatgcctgagtaatgtgtaggtaaagattcaaacgggtgagaaaggccggagacagtcaaatcaccatcaatatgatattcaaccgttctagctgataaattcatgccggagagggtagctatttttgagaggtctctacaaaggctatcaggtcattgcctgagagtctggagcaaacaagagaatcgatgaacggtaatcgtaaaactagcatgtcaatcatatgtaccccggttgataatcagaaaagccccaaaaacaggaagattgtataagcaaatatttaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaatcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgagcacgtataacgtgctttcctcgttagaatcagagcgggagctaaacaggaggccgattaaagggattttagacaggaacggtacgccagaatcctgagaagtgtttttataatcagtgaggccaccgagtaaaagagtctgtccatcacgcaaattaaccgttgtcgcaatacttctttgattagtaataacatcacttgcctgagtagaagaactcaaactatcggccttgctggtaatatccagaacaatattaccgccagccattgcaacggaatcgccattcgccattcaggctgcgcaactgttgggaagggcgatcggtgcgggcctcttcgctattacgccagct

SEQ ID NO: 2 - DNA, 6766 bp, Adeno-associated virus
  source 1..6766: mol_type = genomic DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttaatacgactcactatagggccgcaccggtaagcttttcacgcgtggatctgaattcaattcacgcgtggtacctctggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaatgaattcgccaccatgagcgccgccacccactcgcccatgatgcaggtggcgtccggcaacggtgaccgcgaccctttgccccccggatgggagatcaagatcgacccgcagaccggctggcccttcttcgtggaccacaacagccgcaccactacgtggaacgacccgcgcgtgccctctgagggccccaaggagactccatcctctgccaatggcccttcccgggagggctctaggctgccgcctgctagggaaggccaccctgtgtacccccagctccgaccaggctacattcccattcctgtgctccatgaaggcgctgagaaccggcaggtgcaccctttccatgtctatccccagcctgggatgcagcgattccgaactgaggcggcagcagcggctcctcagaggtcccagtcacctctgcggggcatgccagaaaccactcagccagataaacagtgtggacaggtggcagcggcggcggcagcccagcccccagcctcccacggacctgagcggtcccagtctccagctgcctctgactgctcatcctcatcctcctcggccagcctgccttcctccggcaggagcagcctgggcagtcaccagctcccgcgggggtacatctccattccggtgatacacgagcagaacgttacccggccagcagcccagccctccttccaccaagcccagaagacgcactacccagcgcagcagggggagtaccagacccaccagcctgtgtaccacaagatccagggggatgactgggagccccggcccctgcgggcggcatccccgttcaggtcatctgtccagggtgcatcgagccgggagggctcaccagccaggagcagcacgccactccactccccctcgcccatccgtgtgcacaccgtggtcgacaggcctcagcagcccatgacccatcgagaaactgcacctgtttcccagcctgaaaacaaaccagaaagtaagccaggcccagttggaccagaactccctcctggacacatcccaattcaagtgatccgcaaagaggtggattctaaacctgtttcccagaagcccccacctccctctgagaaggtagaggtgaaagttccccctgctccagttccttgtcctcctcccagccctggcccttctgctgtcccctcttcccccaagagtgtggctacagaagagagggcagcccccagcactgcccctgcagaagctacacctccaaaaccaggagaagccgaggctcccccaaaacatccaggagtgctgaaagtggaagccatcctggagaaggtgcaggggctggagcaggctgtagacaactttgaaggcaagaagactgacaaaaagtacctgatgatcgaagagtatttgaccaaagagctgctggccctggattcagtggaccccgagggacgagccgatgtgcgtcaggccaggagagacggtgtcaggaaggttcagaccatcttggaaaaacttgaacagaaagccattgatgtcccaggtcaagtccaggtctatgaactccagcccagcaaccttgaagcagatcagccactgcaggcaatcatggagatgggtgccgtggcagcagacaagggcaagaaaaatgctggaaatgcagaagatccccacacagaaacccagcagccagaagccacagcagcagcgacttcaaaccccagcagcatgacagacacccctggtaacccagcagcaccgtgaggcggccgcggggatccagacatgataagatacattgatgagtttggacaaaccacaactagaatgcagtgaaaaaaatgctttatttgtaaaatttgtgatgctattgctttatttgtaaccattataagctgcaataaacaagttaacaacaacaattgcattcattttatgtttcaggttcagggggaggtgtgggaggttttttcggatcctctagagtcgaccagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaaaatattccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaaccggtaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaacttccaacatccaataaatcatacaggcaaggcaaagaattagcaaaattaagcaataaagcctcagagcataaagctaaatcggttgtaccaaaaacattatgaccctgtaatacttttgcgggagaagcctttatttcaacgcaaggataaaaatttttagaaccctcatatattttaaatgcaatgcctgagtaatgtgtaggtaaagattcaaacgggtgagaaaggccggagacagtcaaatcaccatcaatatgatattcaaccgttctagctgataaattcatgccggagagggtagctatttttgagaggtctctacaaaggctatcaggtcattgcctgagagtctggagcaaacaagagaatcgatgaacggtaatcgtaaaactagcatgtcaatcatatgtaccccggttgataatcagaaaagccccaaaaacaggaagattgtataagcaaatatttaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaatcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgagcacgtataacgtgctttcctcgttagaatcagagcgggagctaaacaggaggccgattaaagggattttagacaggaacggtacgccagaatcctgagaagtgtttttataatcagtgaggccaccgagtaaaagagtctgtccatcacgcaaattaaccgttgtcgcaatacttctttgattagtaataacatcacttgcctgagtagaagaactcaaactatcggccttgctggtaatatccagaacaatattaccgccagccattgcaacggaatcgccattcgccattcaggctgcgcaactgttgggaagggcgatcggtgcgggcctcttcgctattacgccagct

SEQ ID NO: 3 - DNA, 1728 bp, Adeno-associated virus
  source 1..1728: mol_type = genomic DNA
atgagcgccgccacccactcgcccatgatgcaggtggcgtccggcaacggtgaccgcgaccctttgccccccggatgggagatcaagatcgacccgcagaccggctggcccttcttcgtggaccacaacagccgcaccactacgtggaacgacccgcgcgtgccctctgagggccccaaggagactccatcctctgccaatggcccttcccgggagggctctaggctgccgcctgctagggaaggccaccctgtgtacccccagctccgaccaggctacattcccattcctgtgctccatgaaggcgctgagaaccggcaggtgcaccctttccatgtctatccccagcctgggatgcagcgattccgaactgaggcggcagcagcggctcctcagaggtcccagtcacctctgcggggcatgccagaaaccactcagccagataaacagtgtggacaggtggcagcggcggcggcagcccagcccccagcctcccacggacctgagcggtcccagtctccagctgcctctgactgctcatcctcatcctcctcggccagcctgccttcctccggcaggagcagcctgggcagtcaccagctcccgcgggggtacatctccattccggtgatacacgagcagaacgttacccggccagcagcccagccctccttccaccaagcccagaagacgcactacccagcgcagcagggggagtaccagacccaccagcctgtgtaccacaagatccagggggatgactgggagccccggcccctgcgggcggcatccccgttcaggtcatctgtccagggtgcatcgagccgggagggctcaccagccaggagcagcacgccactccactccccctcgcccatccgtgtgcacaccgtggtcgacaggcctcagcagcccatgacccatcgagaaactgcacctgtttcccagcctgaaaacaaaccagaaagtaagccaggcccagttggaccagaactccctcctggacacatcccaattcaagtgatccgcaaagaggtggattctaaacctgtttcccagaagcccccacctccctctgagaaggtagaggtgaaagttccccctgctccagttccttgtcctcctcccagccctggcccttctgctgtcccctcttcccccaagagtgtggctacagaagagagggcagcccccagcactgcccctgcagaagctacacctccaaaaccaggagaagccgaggctcccccaaaacatccaggagtgctgaaagtggaagccatcctggagaaggtgcaggggctggagcaggctgtagacaactttgaaggcaagaagactgacaaaaagtacctgatgatcgaagagtatttgaccaaagagctgctggccctggattcagtggaccccgagggacgagccgatgtgcgtcaggccaggagagacggtgtcaggaaggttcagaccatcttggaaaaacttgaacagaaagccattgatgtcccaggtcaagtccaggtctatgaactccagcccagcaaccttgaagcagatcagccactgcaggcaatcatggagatgggtgccgtggcagcagacaagggcaagaaaaatgctggaaatgcagaagatccccacacagaaacccagcagccagaagccacagcagcagcgacttcaaaccccagcagcatgacagacacccctggtaacccagcagcaccgtga

SEQ ID NO: 4 - DNA, 1728 bp, Adeno-associated virus; identical to SEQ ID NO: 3
  source 1..1728: mol_type = genomic DNA

SEQ ID NO: 5 - protein, 575 aa, Homo sapiens
MSAATHSPMMQVASGNGDRDPLPPGWEIKIDPQTGWPFFVDHNSRTTTWNDPRVPSEGPKETPSSANGPSREGSRLPPAREGHPVYPQLRPGYIPIPVLHEGAENRQVHPFHVYPQPGMQRFRTEAAAAAPQRSQSPLRGMPETTQPDKQCGQVAAAAAAQPPASHGPERSQSPAASDCSSSSSSASLPSSGRSSLGSHQLPRGYISIPVIHEQNVTRPAAQPSFHQAQKTHYPAQQGEYQTHQPVYHKIQGDDWEPRPLRAASPFRSSVQGASSREGSPARSSTPLHSPSPIRVHTVVDRPQQPMTHRETAPVSQPENKPESKPGPVGPELPPGHIPIQVIRKEVDSKPVSQKPPPPSEKVEVKVPPAPVPCPPPSPGPSAVPSSPKSVATEERAAPSTAPAEATPPKPGEAEAPPKHPGVLKVEAILEKVQGLEQAVDNFEGKKTDKKYLMIEEYLTKELLALDSVDPEGRADVRQARRDGVRKVQTILEKLEQKAIDVPGQVQVYELQPSNLEADQPLQAIMEMGAVAADKGKKNAGNAEDPHTETQQPEATAAATSNPSSMTDTPGNPAAP

SEQ ID NO: 6 - DNA, 3245 bp, Adeno-associated virus
  source 1..3245: mol_type = genomic DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttaatacgactcactatagggccgcaccggtaagcttccactacgggtctaggctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtggatccactacgggtctaggctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtggatccactacgggtctaggctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtggatcctccctggggacagcccctcctggctagtcacaccctgtaggctcctctatataacccaggggcacaggggctgcccccgggtcacggtggatatccctgcaggtaccacgcgctgtaattgaactgggagtggacacctgtggagagaaaggcaaagtggatgtcagtaagaccaataggtgcctatcagaaacgcaagagtcttctctgtctcgacaagcccagtttctattggtctccttaaacctgtcttgtaaccttgatacttacggagagcaactgcataagggctagcctcgagaattcgccaccatgagcgccgccacccactcgcccatgatgcaggtggcgtccggcaacggtgaccgcgaccctttgccccccggatgggagatcaagatcgacccgcagaccggctggcccttcttcgtggaccacaacagccgcaccactacgtggaacgacccgcgcgtgccctctgagggccccaaggagactccatcctctgccaatggcccttcccgggagggctctaggctgccgcctgctagggaaggccaccctgtgtacccccagctccgaccaggctacattcccattcctgtgctccatgaaggcgctgagaaccggcaggtgcaccctttccatgtctatccccagcctgggatgcagcgattccgaactgaggcggcagcagcggctcctcagaggtcccagtcacctctgcggggcatgccagaaaccactcagccagataaacagtgtggacaggtggcagcggcggcggcagcccagcccccagcctcccacggacctgagcggtcccagtctccagctgcctctgactgctcatcctcatcctcctcggccagcctgccttcctccggcaggagcagcctgggcagtcaccagctcccgcgggggtacatctccattccggtgatacacgagcagaacgttacccggccagcagcccagccctccttccaccaagcccagaagacgcactacccagcgcagcagggggagtaccagacccaccagcctgtgtaccacaagatccagggggatgactgggagccccggcccctgcgggcggcatccccgttcaggtcatctgtccagggtgcatcgagccgggagggctcaccagccaggagcagcacgccactccactccccctcgcccatccgtgtgcacaccgtggtcgacaggcctcagcagcccatgacccatcgagaaactgcacctgtttcccagcctgaaaacaaaccagaaagtaagccaggcccagttggaccagaactccctcctggacacatcccaattcaagtgatccgcaaagaggtggattctaaacctgtttcccagaagcccccacctccctctgagaaggtagaggtgaaagttccccctgctccagttccttgtcctcctcccagccctggcccttctgctgtcccctcttcccccaagagtgtggctacagaagagagggcagcccccagcactgcccctgcagaagctacacctccaaaaccaggagaagccgaggctcccccaaaacatccaggagtgctgaaagtggaagccatcctggagaaggtgcaggggctggagcaggctgtagacaactttgaaggcaagaagactgacaaaaagtacctgatgatcgaagagtatttgaccaaagagctgctggccctggattcagtggaccccgagggacgagccgatgtgcgtcaggccaggagagacggtgtcaggaaggttcagaccatcttggaaaaacttgaacagaaagccattgatgtcccaggtcaagtccaggtctatgaactccagcccagcaaccttgaagcagatcagccactgcaggcaatcatggagatgggtgccgtggcagcagacaagggcaagaaaaatgctggaaatgcagaagatccccacacagaaacccagcagccagaagccacagcagcagcgacttcaaaccccagcagcatgacagacacccctggtaacccagcagcaccgtgaggcggccgcggggatccagacatgataagatacattgatgagtttggacaaaccacaactagaatgcagtgaaaaaaatgctttatttgtaaaatttgtgatgctattgctttatttgtaaccattataagctgcaataaacaagttaacaacaacaattgcattcattttatgtttcaggttcagggggaggtgtgggaggttttttcggatcctctagagtcgaccagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 7 - DNA, 3114 bp, Adeno-associated virus
  source 1..3114: mol_type = genomic DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttaatacgactcactatagggccgcaccggtaagcttttcacgcgtggatctgaattcaattcacgcgtggtacctctggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaatgaattcgccaccatgagcgccgccacccactcgcccatgatgcaggtggcgtccggcaacggtgaccgcgaccctttgccccccggatgggagatcaagatcgacccgcagaccggctggcccttcttcgtggaccacaacagccgcaccactacgtggaacgacccgcgcgtgccctctgagggccccaaggagactccatcctctgccaatggcccttcccgggagggctctaggctgccgcctgctagggaaggccaccctgtgtacccccagctccgaccaggctacattcccattcctgtgctccatgaaggcgctgagaaccggcaggtgcaccctttccatgtctatccccagcctgggatgcagcgattccgaactgaggcggcagcagcggctcctcagaggtcccagtcacctctgcggggcatgccagaaaccactcagccagataaacagtgtggacaggtggcagcggcggcggcagcccagcccccagcctcccacggacctgagcggtcccagtctccagctgcctctgactgctcatcctcatcctcctcggccagcctgccttcctccggcaggagcagcctgggcagtcaccagctcccgcgggggtacatctccattccggtgatacacgagcagaacgttacccggccagcagcccagccctccttccaccaagcccagaagacgcactacccagcgcagcagggggagtaccagacccaccagcctgtgtaccacaagatccagggggatgactgggagccccggcccctgcgggcggcatccccgttcaggtcatctgtccagggtgcatcgagccgggagggctcaccagccaggagcagcacgccactccactccccctcgcccatccgtgtgcacaccgtggtcgacaggcctcagcagcccatgacccatcgagaaactgcacctgtttcccagcctgaaaacaaaccagaaagtaagccaggcccagttggaccagaactccctcctggacacatcccaattcaagtgatccgcaaagaggtggattctaaacctgtttcccagaagcccccacctccctctgagaaggtagaggtgaaagttccccctgctccagttccttgtcctcctcccagccctggcccttctgctgtcccctcttcccccaagagtgtggctacagaagagagggcagcccccagcactgcccctgcagaagctacacctccaaaaccaggagaagccgaggctcccccaaaacatccaggagtgctgaaagtggaagccatcctggagaaggtgcaggggctggagcaggctgtagacaactttgaaggcaagaagactgacaaaaagtacctgatgatcgaagagtatttgaccaaagagctgctggccctggattcagtggaccccgagggacgagccgatgtgcgtcaggccaggagagacggtgtcaggaaggttcagaccatcttggaaaaacttgaacagaaagccattgatgtcccaggtcaagtccaggtctatgaactccagcccagcaaccttgaagcagatcagccactgcaggcaatcatggagatgggtgccgtggcagcagacaagggcaagaaaaatgctggaaatgcagaagatccccacacagaaacccagcagccagaagccacagcagcagcgacttcaaaccccagcagcatgacagacacccctggtaacccagcagcaccgtgaggcggccgcggggatccagacatgataagatacattgatgagtttggacaaaccacaactagaatgcagtgaaaaaaatgctttatttgtaaaatttgtgatgctattgctttatttgtaaccattataagctgcaataaacaagttaacaacaacaattgcattcattttatgtttcaggttcagggggaggtgtgggaggttttttcggatcctctagagtcgaccagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 8 - DNA, 2561 bp, synthetic construct
  source 1..2561: mol_type = other DNA
gcatccaaccccgggccgcggccaacttctctggactggaccagaagtttctagccggccagttgctacctccctttatctcctccttcccctctggcagcgaggaggctatttccagacacttccacccctctctggccacgtcacccccgcctttaattcataaaggtgcccggcgccggcttcccggacacgtcggcggcggagaggggcccacggcggcggcccggccagagactcggcgcccggagccagcgccccgcacccgcgccccagcgggcagaccccaacccagcatgagcgccgccacccactcgcccatgatgcaggtggcgtccggcaacggtgaccgcgaccctttgccccccggatgggagatcaagatcgacccgcagaccggctggcccttcttcgtggaccacaacagccgcaccactacgtggaacgacccgcgcgtgccctctgagggccccaaggagactccatcctctgccaatggcccttcccgggagggctctaggctgccgcctgctagggaaggccaccctgtgtacccccagctccgaccaggctacattcccattcctgtgctccatgaaggcgctgagaaccggcaggtgcaccctttccatgtctatccccagcctgggatgcagcgattccgaactgaggcggcagcagcggctcctcagaggtcccagtcacctctgcggggcatgccagaaaccactcagccagataaacagtgtggacaggtggcagcggcggcggcagcccagcccccagcctcccacggacctgagcggtcccagtctccagctgcctctgactgctcatcctcatcctcctcggccagcctgccttcctccggcaggagcagcctgggcagtcaccagctcccgcgggggtacatctccattccggtgatacacgagcagaacgttacccggccagcagcccagccctccttccaccaagcccagaagacgcactacccagcgcagcagggggagtaccagacccaccagcctgtgtaccacaagatccagggggatgactgggagccccggcccctgcgggcggcatccccgttcaggtcatctgtccagggtgcatcgagccgggagggctcaccagccaggagcagcacgccactccactccccctcgcccatccgtgtgcacaccgtggtcgacaggcctcagcagcccatgacccatcgagaaactgcacctgtttcccagcctgaaaacaaaccagaaagtaagccaggcccagttggaccagaactccctcctggacacatcccaattcaagtgatccgcaaagaggtggattctaaacctgtttcccagaagcccccacctccctctgagaaggtagaggtgaaagttccccctgctccagttccttgtcctcctcccagccctggcccttctgctgtcccctcttcccccaagagtgtggctacagaagagagggcagcccccagcactgcccctgcagaagctacacctccaaaaccaggagaagccgaggctcccccaaaacatccaggagtgctgaaagtggaagccatcctggagaaggtacaggggctggagcaggctgtagacaactttgaaggcaagaagactgacaaaaagtacctgatgatcgaagagtatttgaccaaagagctgctggccctggattcagtggaccccgagggacgagccgatgtgcgtcaggccaggagagacggtgtcaggaaggttcagaccatcttggaaaaacttgaacagaaagccattgatgtcccaggtcaagtccaggtctatgaactccagcccagcaaccttgaagcagatcagccactgcaggcaatcatggagatgggtgccgtggcagcagacaagggcaagaaaaatgctggaaatgcagaagatccccacacagaaacccagcagccagaagccacagcagcagcgacttcaaaccccagcagcatgacagacacccctggtaacccagcagcaccgtagcctctgccctgtaaaaatcagactcggaaccgatgtgtgctttagggaattttaagttgcatgcatttcagagactttaagtcagttggtttttattagctgcttggtatgcagtaacttgggtggaggcaaaacactaataaaagggctaaaaaggaaaatgatgcttttcttctatattcttactctgtacaaataaagaagttgcttgttgtttgagaagtttaaccccgttgcttgttgttctgcagccctgtctacttgggcacccccaccacctgttagctgtggttgtgcactgtcttttgtagctctggactggaggggtagatggggagtcaattacccatcacataaatatgaaacatttatcagaaatgttgccattttaatgagatgattttcttcatctcataattaaaatacctgactttagagagagtaaaatgtgccaggagccataggaatatctgtatgttggatgactttaatgctacattttaaaaaaagaaaataaagtaataatataactcaaaa

SEQ ID NO: 9 - DNA, 774 bp, Homo sapiens
  source 1..774: mol_type = genomic DNA; note = hNTF3
atgtccatcttgttttatgtgatatttctcgcttatctccgtggcatccaaggtaacaacatggatcaaaggagtttgccagaagactcgctcaattccctcattattaagctgatccaggcagatattttgaaaaacaagctctccaagcagatggtggacgttaaggaaaattaccagagcaccctgcccaaagctgaggctccccgagagccggagcggggagggcccgccaagtcagcattccagccggtgattgcaatggacaccgaactgctgcgacaacagagacgctacaactcaccgcgggtcctgctgagcgacagcacccccttggagcccccgcccttgtatctcatggaggattacgtgggcagccccgtggtggcgaacagaacatcacggcggaaacggtacgcggagcataagagtcaccgaggggagtactcggtatgtgacagtgagagtctgtgggtgaccgacaagtcatcggccatcgacattcggggacaccaggtcacggtgctgggggagatcaaaacgggcaactctcccgtcaaacaatatttttatgaaacgcgatgtaaggaagccaggccggtcaaaaacggttgcaggggtattgatgataaacactggaactctcagtgcaaaacatcccaaacctacgtccgagcactgacttcagagaacaataaactcgtgggctggcggtggatacggatagacacgtcctgtgtgtgtgccttgtcgagaaaaatcggaagaacatga

SEQ ID NO: 10 - protein, 270 aa, Homo sapiens
  source 1..270: note = NT-3 amino acid sequence
MVTFATILQVNKVMSILFYVIFLAYLRGIQGNNMDQRSLPEDSLNSLIIKLIQADILKNKLSKQMVDVKENYQSTLPKAEAPREPERGGPAKSAFQPVIAMDTELLRQQRRYNSPRVLLSDSTPLEPPPLYLMEDYVGSPVVANRTSRRKRYAEHKSHRGEYSVCDSESLWVTDKSSAIDIRGHQVTVLGEIKTGNSPVKQYFYETRCKEARPVKNGCRGIDDKHWNSQCKTSQTYVRALTSENNKLVGWRWIRIDTSCVCALSRKIGRT

SEQ ID NO: 11 - DNA, 714 bp, synthetic construct
  source 1..714: mol_type = other DNA; note = tMCK Promoter
ccactacgggtctaggctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtggatccactacgggtctatgctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtggaccactacgggtctaggctgcccatgtaaggaggcaaggcctggggacacccgagatgcctggttataattaaccccaacacctgctgccccccccccccaacacctgctgcctgagcctgagcggttaccccaccccggtgcctgggtcttaggctctgtacaccatggaggagaagctcgctctaaaaataaccctgtccctggtcctccctggggacagcccctcctggctagtcacaccctgtaggctcctctatataacccaggggcacaggggctgcccccgggtcac

SEQ ID NO: 12 - DNA, 5884 bp, synthetic construct
  source 1..5884: mol_type = other DNA; note = scpAAV.tmCK.NTF3 plasmid genome full sequence
cagcagctgcgcgctcgctcgctcactgaggccgcccgggcaaagcccgggcgtcgggcgacctttggtcgcccggcctcagtgagcgagcgagcgcgcagagagggagtggggttaaccaattggcggccgcaaacttgcatgccccactacgggtctaggctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtggatccactacgggtctatgctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtggaccactacgggtctaggctgcccatgtaaggaggcaaggcctggggacacccgagatgcctggttataattaaccccaacacctgctgccccccccccccaacacctgctgcctgagcctgagcggttaccccaccccggtgcctgggtcttaggctctgtacaccatggaggagaagctcgctctaaaaataaccctgtccctggtcctccctggggacagcccctcctggctagtcacaccctgtaggctcctctatataacccaggggcacaggggctgcccccgggtcacctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagcgcgtggtacctgcagggatatccaccatgtccatcttgttttatgtgatatttctcgcttatctccgtggcatccaaggtaacaacatggatcaaaggagtttgccagaagactcgctcaattccctcattattaagctgatccaggcagatattttgaaaaacaagctctccaagcagatggtggacgttaaggaaaattaccagagcaccctgcccaaagctgaggctccccgagagccggagcggggagggcccgccaagtcagcattccagccggtgattgcaatggacaccgaactgctgcgacaacagagacgctacaactcaccgcgggtcctgctgagcgacagcacccccttggagcccccgcccttgtatctcatggaggattacgtgggcagccccgtggtggcgaacagaacatcacggcggaaacggtacgcggagcataagagtcaccgaggggagtactcggtatgtgacagtgagagtctgtgggtgaccgacaagtcatcggccatcgacattcggggacaccaggtcacggtgctgggggagatcaaaacgggcaactctcccgtcaaacaatatttttatgaaacgcgatgtaaggaagccaggccggtcaaaaacggttgcaggggtattgatgataaacactggaactctcagtgcaaaacatcccaaacctacgtccgagcactgacttcagagaacaataaactcgtgggctggcggtggatacggatagacacgtcctgtgtgtgtgccttgtcgagaaaaatcggaagaacatgaggcggccgcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggcgcgcctctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggaattccagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaattcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 13 - DNA, 6714 bp, Adeno-associated virus
  source 1..6714: mol_type = genomic DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgccaccatgagcgccgccacccactcgcccatgatgcaggtggcgtccggcaacggtgaccgcgaccctttgccccccggatgggagatcaagatcgacccgcagaccggctggcccttcttcgtggaccacaacagccgcaccactacgtggaacgacccgcgcgtgccctctgagggccccaaggagactccatcctctgccaatggcccttcccgggagggctctaggctgccgcctgctagggaaggccaccctgtgtacccccagctccgaccaggctacattcccattcctgtgctccatgaaggcgctgagaaccggcaggtgcaccctttccatgtctatccccagcctgggatgcagcgattccgaactgaggcggcagcagcggctcctcagaggtcccagtcacctctgcggggcatgccagaaaccactcagccagataaacagtgtggacaggtggcagcggcggcggcagcccagcccccagcctcccacggacctgagcggtcccagtctccagctgcctctgactgctcatcctcatcctcctcggccagcctgccttcctccggcaggagcagcctgggcagtcaccagctcccgcgggggtacatctccattccggtgatacacgagcagaacgttacccggccagcagcccagccctccttccaccaagcccagaagacgcactacccagcgcagcagggggagtaccagacccaccagcctgtgtaccacaagatccagggggatgactgggagccccggcccctgcgggcggcatccccgttcaggtcatctgtccagggtgcatcgagccgggagggctcaccagccaggagcagcacgccactccactccccctcgcccatccgtgtgcacaccgtggtcgacaggcctcagcagcccatgacccatcgagaaactgcacctgtttcccagcctgaaaacaaaccagaaagtaagccaggcccagttggaccagaactccctcctggacacatcccaattcaagtgatccgcaaagaggtggattctaaacctgtttcccagaagcccccacctccctctgagaaggtagaggtgaaagttccccctgctccagttccttgtcctcctcccagccctggcccttctgctgtcccctcttcccccaagagtgtggctacagaagagagggcagcccccagcactgcccctgcagaagctacacctccaaaaccaggagaagccgaggctcccccaaaacatccaggagtgctgaaagtggaagccatcctggagaaggtgcaggggctggagcaggctgtagacaactttgaaggcaagaagactgacaaaaagtacctgatgatcgaagagtatttgaccaaagagctgctggccctggattcagtggaccccgagggacgagccgatgtgcgtcaggccaggagagacggtgtcaggaaggttcagaccatcttggaaaaacttgaacagaaagccattgatgtcccaggtcaagtccaggtctatgaactccagcccagcaaccttgaagcagatcagccactgcaggcaatcatggagatgggtgccgtggcagcagacaagggcaagaaaaatgctggaaatgcagaagatccccacacagaaacccagcagccagaagccacagcagcagcgacttcaaaccccagcagcatgacagacacccctggtaacccagcagcaccgtgaggcggccgcggggatccagacatgataagatacattgatgagtttggacaaaccacaactagaatgcagtgaaaaaaatgctttatttgtaaaatttgtgatgctattgctttatttgtaaccattataagctgcaataaacaagttaacaacaacaattgcattcattttatgtttcaggttcagggggaggtgtgggaggttttttcggatcctctagagtcgaccagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtacgcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaacttccaacatccaataaatcatacaggcaaggcaaagaattagcaaaattaagcaataaagcctcagagcataaagctaaatcggttgtaccaaaaacattatgaccctgtaatacttttgcgggagaagcctttatttcaacgcaaggataaaaatttttagaaccctcatatattttaaatgcaatgcctgagtaatgtgtaggtaaagattcaaacgggtgagaaaggccggagacagtcaaatcaccatcaatatgatattcaaccgttctagctgataaattcatgccggagagggtagctatttttgagaggtctctacaaaggctatcaggtcattgcctgagagtctggagcaaacaagagaatcgatgaacggtaatcgtaaaactagcatgtcaatcatatgtaccccggttgataatcagaaaagccccaaaaacaggaagattgtataagcaaatatttaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaatcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgagcacgtataacgtgctttcctcgttagaatcagagcgggagctaaacaggaggccgattaaagggattttagacaggaacggtacgccagaatcctgagaagtgtttttataatcagtgaggccaccgagtaaaagagtctgtccatcacgcaaattaaccgttgtcgcaatacttctttgattagtaataacatcacttgcctgagtagaagaactcaaactatcggccttgctggtaatatccagaacaatattaccgccagccattgcaacggaatcgccattcgccattcaggctgcgcaactgttgggaagggcgatcggtgcgggcctcttcgctattacgccagct